SEQUENCE LISTING (6 sequences)

SEQ ID NO: 1 - DNA, 18 bp, synthetic construct
  source 1..18: mol_type = other DNA
  modified_base 1: mod_base = OTHER; note = 5' phosphorylated nucleotide
ggtgcgtttttcgcacct

SEQ ID NO: 2 - DNA, 19 bp, synthetic construct
  source 1..19: mol_type = other DNA
aggtgcgtttttcgcacct

SEQ ID NO: 3 - DNA, 12 bp, synthetic construct
  source 1..12: mol_type = other DNA
cctgtcacctgg

SEQ ID NO: 4 - DNA, 29 bp, synthetic construct
  source 1..29: mol_type = other DNA
ggtccactgtcctttttggacagtggacc

SEQ ID NO: 5 - DNA, 26 bp, synthetic construct
  source 1..26: mol_type = other DNA
ccatctcatccctgcgtgtctccgac

SEQ ID NO: 6 - DNA, 57 bp, synthetic construct
  source 1..57: mol_type = other DNA
cagcctctgtgcgtccctactctacctttttggtagagtagggacgcacagaggctg

(19) United States
(12) Patent Application Publication
(10) Pub. No.: US 2023/0340570 A1
(43) Pub. Date: Oct. 26, 2023

(54) METHODS AND SYSTEMS FOR REDUCING PARTICLE AGGREGATION

(71) Applicant: Ultima Genomics, Inc., Newark, CA (US)

(72) Inventors: Daniel MAZUR, San Diego, CA (US); Kevin HEINEMANN, Carlsbad, CA (US); Theo NIKIFOROV, Carlsbad, CA (US); Robert ONO, Palo Alto, CA (US); Aklilu WORKU, San Francisco, CA (US); Florian OBERSTRASS, Menlo Park, CA (US)

(21) Appl. No.: 18/114,091

(22) Filed: Feb. 24, 2023

Related U.S. Application Data
(63) Continuation of application No. PCT/US2021/048523, filed on Aug. 31, 2021.
(60) Provisional application No. 63/073,305, filed on Sep. 1, 2020.

(51) Int. Cl. C12Q 1/6811

(52) U.S. Cl. CPC C12Q 1/6811

(57) ABSTRACT

The present disclosure provides methods, systems, compositions, and kits for reducing particle aggregation and improving sequencing quality. A method for processing particles may comprise the use of a first buffer solution or a second buffer solution. A method for processing particles may comprise the use of a single-strand binding moiety such as a single-strand binding protein.

### CROSS REFERENCE

This application is a continuation of International Patent Application No. PCT/US2021/048523, filed on Aug. 31, 2021, which claims the benefit of U.S. Provisional Pat. Application No. 63/073,305, filed on Sep. 01, 2020, which is hereby incorporated by reference in its entirety.

### BACKGROUND

The detection, quantification and sequencing of cells and biological molecules may be important for molecular biology and medical applications, such as diagnostics. Genetic testing may be useful for a number of diagnostic methods. For example, disorders that are caused by rare genetic alterations (e.g., sequence variants) or changes in epigenetic markers, such as cancer and partial or complete aneuploidy, may be detected or more accurately characterized with deoxyribonucleic acid (DNA) or ribonucleic acid (RNA) sequence information.

Nucleic acid sequencing is a process that can be used to provide sequence information for a nucleic acid sample. Such sequence information may be helpful in diagnosing and/or treating a subject with a condition. For example, the nucleic acid sequence of a subject may be used to identify, diagnose and potentially develop treatments for genetic diseases. As another example, research into pathogens may lead to treatment of contagious diseases.

Nucleic acid sequencing may comprise sequencing of nucleic acid molecules coupled to particles such as beads. However, the use of such systems may reduce sequencing quality via, for example, aggregation of particles via associations between nucleic acid molecules bound to the particles.

### SUMMARY

The present disclosure provides methods, systems, compositions, and kits that may be useful for reducing particle aggregation and/or improving sequencing quality in nucleic acid sequencing processes. For example, the present disclosure provides methods, systems, compositions, and kits for treating particles such that nucleic acid molecules coupled to the particles may not associate and generate particle aggregates. The present disclosure also provides methods, systems, compositions, and kits for stable, long-term storage of particles comprising nucleic acid molecules. Particles processed according to the methods, systems, compositions, and kits provided herein may be useful in nucleic acid sequencing applications.

Disclosed herein, are methods for processing a plurality of particles. In an aspect, a method for processing a plurality of particles comprises: (a) providing a first particle of the plurality of particles comprising a first nucleic acid molecule immobilized thereto, wherein the first nucleic acid molecule comprises a first single-stranded portion; (b) contacting the first nucleic acid molecule with a first single-stranded binding moiety under conditions sufficient to couple the first single-stranded binding moiety to the first single-stranded portion of the first nucleic acid molecule to yield a first treated particle comprising a first blocked nucleic acid molecule immobilized thereto, wherein the first blocked nucleic acid molecule comprises the first nucleic acid molecule coupled to the first single-stranded binding moiety; and (c) providing the first treated particle in a solution comprising a second treated particle, wherein the second treated particle comprises a second nucleic acid molecule comprising a second single-stranded portion coupled to a second single-stranded binding moiety.

In some embodiments, the plurality of particles is a plurality of beads. In some embodiments, the first nucleic acid molecule comprises deoxyribonucleic acid (DNA) nucleotides, ribonucleic acid (RNA) nucleotides, or a combination thereof. In some embodiments, the first single-stranded portion comprises single-stranded deoxyribonucleic acid (ssDNA), ribonucleic acid (RNA), or a combination thereof. In some embodiments, the first nucleic acid molecule comprises a sequence of a sample nucleic acid molecule, or a complement thereof. In some embodiments, the first nucleic acid molecule comprises a priming sequence, or a complement thereof. In some embodiments, the priming sequence is a targeted priming sequence. In some embodiments, the priming sequence comprises a random N-mer sequence. In some embodiments, the first nucleic acid molecule comprises a barcode sequence or a unique molecular identifier sequence.

In some embodiments, (b) comprises providing a reaction mixture comprising the first single-stranded binding moiety. In some embodiments, the reaction mixture comprises a salt. In some embodiments, the reaction mixture comprises spermine. In some embodiments, the reaction mixture comprises cobalt hexammine.

In some embodiments, the first single-stranded binding moiety comprises a single-stranded binding (SSB) protein. In some embodiments, the SSB protein is a T4 phage-derived SSB protein, an Escherichia coli-derived SSB protein, or an Extreme Thermostable SSB protein. In some embodiments, the single-stranded binding moiety comprises a third nucleic acid molecule. In some embodiments, the third nucleic acid molecule comprises a random N-mer. In some embodiments, N is between 6 and 12. In some embodiments, the second nucleic acid molecule comprises 6 bases. In some embodiments, the third nucleic acid molecule has sequence complementarity to a sequence of the first single-stranded portion of the first nucleic acid molecule. In some embodiments, the first single-stranded binding moiety comprises a loop structure. In some embodiments, the loop structure is included in a hairpin moiety. In some embodiments, the first single-stranded binding moiety comprises a single-stranded nucleic acid molecule coupled to a moiety comprising the loop structure.

In some embodiments, the first particle comprises a first plurality of nucleic acid molecules immobilized thereto, wherein the first plurality of nucleic acid molecules comprises the nucleic acid molecule, and wherein the first plurality of nucleic acid molecules has at least partial sequence identity to a first nucleic acid sequence. In some embodiments, the second treated particle comprises a second plurality of nucleic acid molecules immobilized thereto, wherein the second plurality of nucleic acid molecules is different from the first plurality of nucleic acid molecules, and wherein the second plurality of nucleic acid molecules has at least partial sequence identity to a second nucleic acid sequence. In some embodiments, the second nucleic acid sequence is different than the first nucleic acid sequence. In some embodiments, the second nucleic acid sequence and the first nucleic acid sequence are identical.

In some embodiments, the first plurality of nucleic acid molecules comprises at least 1,000 nucleic acid molecules. In some embodiments, the first plurality of nucleic acid molecules comprises at least 100,000 nucleic acid molecules. In some embodiments, the plurality of particles comprises at least 10,000,000 particles. In some embodiments, the plurality of particles comprises at least 1,000,000,000 particles. In some embodiments, the first single-stranded binding moiety and the second single-stranded binding moiety are of a same type. In some embodiments, the first single-stranded binding moiety and the second single-stranded binding moiety are of different types.

In some embodiments, the method further comprises immobilizing the first particle to a substrate. In some embodiments, the first particle and the second particle are immobilized to different independently addressable locations of the substrate. In some embodiments, the independently addressable locations are substantially planar. In some embodiments, the independently addressable locations comprise one or more wells. In some embodiments, the independently addressable locations comprise one or more pillars. In some embodiments, the substrate comprises the solution. In some embodiments, (b) comprises contacting the plurality of particles with a plurality of single-stranded binding moieties under conditions sufficient to couple the single-stranded binding moieties of the plurality of single-stranded binding moieties to single-stranded portions of nucleic acid molecules immobilized to particles of the plurality of particles to yield a plurality of treated particles comprising the first treated particle and the second treated particle. In some embodiments, the plurality of single-stranded binding moieties comprises a single type of single-stranded binding moiety. In some embodiments, the plurality of single-stranded binding moieties comprises a plurality of different types of single-stranded binding moieties. In some embodiments, at a given time subsequent to (b), the plurality of treated particles comprises a number of particles that is at least 50% of the number of particles of the plurality of particles. In some embodiments, at a given time subsequent to (b), the plurality of treated particles comprises a number of particles that is at least 70% of the number of particles of the plurality of particles. In some embodiments, at a given time subsequent to (b), the plurality of treated particles comprises a number of particles that is at least 90% of the number of particles of the plurality of particles. In some embodiments, subsequent to (b), particle aggregates, comprising two or more treated particles of the plurality of treated particles, having a dimension of at least about 1 micrometer (µm) are absent from the plurality of treated particles. In some embodiments, subsequent to (b), no more than 1% of treated particles of the plurality of treated particles are included in a particle aggregate comprising two or more treated particles of the plurality of treated particles. In some embodiments, the method further comprises immobilizing the plurality of treated particles to a substrate. In some embodiments, the plurality of treated particles is immobilized to different independently addressable locations of the substrate. In some embodiments, the independently addressable locations are substantially planar. In some embodiments, the independently addressable locations comprise one or more wells. In some embodiments, the independently addressable locations comprise one or more pillars. In some embodiments, the method further comprises, prior to (a), denaturing a double-stranded portion of the first nucleic acid molecule to yield the first single-stranded portion. In some embodiments, the method further comprises sequencing the first single-stranded portion of the first nucleic acid molecule, or a portion thereof.

Disclosed herein, are methods for processing a plurality of particles. In an aspect, a method for processing a plurality of particles comprises: (a) providing the plurality of particles, wherein each particle of at least a subset of the plurality of particles comprises a nucleic acid molecule of a plurality of nucleic acid molecules immobilized thereto, wherein each nucleic acid molecule of at least the subset of the plurality of nucleic acid molecules comprises a single-stranded portion; and (b) contacting the plurality of particles with a plurality of single-stranded binding moieties under conditions sufficient for single-stranded binding moieties of the plurality of single-stranded binding moieties to couple to single-stranded portions of nucleic acid molecules of the at least the subset of the plurality of nucleic acid molecules, wherein, subsequent to (b), the plurality of particles are included in a solution, and wherein no more than 1% of particles of the plurality of particles are included in a particle aggregate comprising two or more particles of the plurality of particles.

In some embodiments, the plurality of particles is a plurality of beads. In some embodiments, the plurality of nucleic acid molecules comprises a plurality of deoxyribonucleic acid (DNA) molecules, a plurality of ribonucleic acid (RNA) molecules, or a combination thereof. In some embodiments, the plurality of nucleic acid molecules comprises sequences of sample nucleic acid molecules, or complements thereof. In some embodiments, the plurality of nucleic acid molecules comprises a plurality of priming sequences, or complements thereof. In some embodiments, the plurality of priming sequences comprises a plurality of targeted priming sequences. In some embodiments, the plurality of priming sequences comprises a plurality of random N-mer sequences.

In some embodiments, (b) comprises providing a reaction mixture comprising the plurality of single-stranded binding moieties. In some embodiments, the reaction mixture comprises a salt. In some embodiments, the reaction mixture comprises spermine. In some embodiments, the reaction mixture comprises cobalt hexammine.

In some embodiments, the plurality of single-stranded binding moieties comprises a plurality of single-stranded binding (SSB) proteins. In some embodiments, the plurality of single-stranded binding (SSB) proteins comprises T4 phage-derived SSB proteins, Escherichia coli-derived SSB proteins, Extreme Thermostable SSB proteins, or a combination thereof. In some embodiments, the plurality of single-stranded binding moieties comprises an additional plurality of nucleic acid molecules. In some embodiments, the additional plurality of nucleic acid molecules comprises a plurality of random N-mers. In some embodiments, the additional plurality of nucleic acid molecules has sequence complementarity to sequences of the single-stranded portions of the at least the subset of the plurality of nucleic acid molecules.

In some embodiments, the additional plurality of nucleic acid molecules comprises a plurality of loop structures. In some embodiments, the plurality of loop structures is included in a plurality of hairpin moieties. In some embodiments, the additional plurality of nucleic acid molecules comprises a plurality of single-stranded nucleic acid molecules coupled to a plurality of moieties comprising the plurality of loop structures. In some embodiments, the plurality of single-stranded binding moieties comprises a single type of single-stranded binding moiety. In some embodiments, the plurality of single-stranded binding moieties comprises a plurality of different types of single-stranded binding moieties. In some embodiments, nucleic acid molecules of the plurality of nucleic acid molecules immobilized to a given particle of the at least the subset of the plurality of particles have sequence identity to a first nucleic acid sequence In some embodiments, additional nucleic acid molecules of the plurality of nucleic acid molecules immobilized to an additional given particle of the at least the subset of the plurality of particles have sequence identity to a second nucleic acid sequence. In some embodiments, the first nucleic acid sequence and the second nucleic acid sequence are identical. In some embodiments, the first nucleic acid sequence and the second nucleic acid sequence are different.

In some embodiments, the plurality of particles comprises at least 10,000,000 particles. In some embodiments, the plurality of particles comprises at least 1,000,000,000 particles. In some embodiments, at a given time subsequent to (b), at least 50% of particles of the plurality of particles comprise a nucleic acid molecule of the plurality of nucleic acid molecules that is coupled to a single-stranded binding moiety of the plurality of single-stranded binding moieties. In some embodiments, at a given time subsequent to (b), at least 70% of particles of the plurality of particles comprise a nucleic acid molecule of the plurality of nucleic acid molecules that is coupled to a single-stranded binding moiety of the plurality of single-stranded binding moieties. In some embodiments, at a given time subsequent to (b), at least 90% of particles of the plurality of particles comprise a nucleic acid molecule of the plurality of nucleic acid molecules that is coupled to a single-stranded binding moiety of the plurality of single-stranded binding moieties. In some embodiments, subsequent to (b), no more than 0.1% of particles of the plurality of particles are included in a particle aggregate comprising two or more particles of the plurality of particles. In some embodiments, subsequent to (b), no more than 0.01% of particles of the plurality of particles are included in a particle aggregate comprising two or more particles of the plurality of particles. In some embodiments, subsequent to (b), no more than 0.001% of particles of the plurality of particles are included in a particle aggregate comprising two or more particles of the plurality of particles.

In some embodiments, particle aggregates, comprising two or more particles of the plurality of particles, having a dimension of at least about 5 micrometer (µm) are absent from the plurality of particles. In some embodiments, particle aggregates, comprising two or more particles of the plurality of particles, having a dimension of at least about 1 micrometer (µm) are absent from the plurality of particles. In some embodiments, the method further comprises immobilizing the plurality of particles to a substrate. In some embodiments, the plurality of particles is immobilized to different independently addressable locations of the substrate. In some embodiments, the independently addressable locations are substantially planar. In some embodiments, the independently addressable locations comprise one or more wells. In some embodiments, the independently addressable locations comprise one or more pillars. In some embodiments, the method further comprises, prior to (a), denaturing double-stranded portions of the at least the subset of the plurality of nucleic acid molecules to yield the single-stranded portions. In some embodiments, the method further comprises sequencing the single-stranded portions of the at least the subset of the plurality of nucleic acid molecules, or portions thereof.

Disclosed herein, are compositions. In an aspect, a composition comprises: a suspension comprising: (i) a plurality of particles comprising a first particle and a second particle, wherein the plurality of particles comprises a plurality of nucleic acid molecules immobilized thereto, wherein the first particle comprises a first nucleic acid molecule of the plurality of nucleic acid molecules immobilized thereto and the second particle comprises a second nucleic acid molecule of the plurality of nucleic acid molecules immobilized thereto, wherein the first nucleic acid molecule comprises a single-stranded portion; and (ii) a single-stranded binding moiety, wherein the single-stranded binding moiety is configured to couple to the single-stranded portion of the first nucleic acid molecule, wherein the first particle is in fluidic communication with the second particle.

In some embodiments, the plurality of particles is a plurality of beads. In some embodiments, the plurality of nucleic acid molecules comprises a plurality of deoxyribonucleic acid (DNA) molecules, ribonucleic acid (RNA) molecules, or a combination thereof. In some embodiments, the single-stranded portion of the first nucleic acid molecule comprises single-stranded deoxyribonucleic acid (ssDNA), ribonucleic acid (RNA), or a combination thereof. In some embodiments, the first nucleic acid molecule comprises a sequence of a sample nucleic acid molecule, or a complement thereof. In some embodiments, the first nucleic acid molecule comprises a priming sequence, or a complement thereof. In some embodiments, the priming sequence is a targeted priming sequence. In some embodiments, the priming sequence comprises a random N-mer sequence. In some embodiments, the first nucleic acid molecule comprises a barcode sequence or a unique molecular identifier sequence.

In some embodiments, the method further comprises one or more reagents for facilitating coupling of the single-stranded binding moiety and the single-stranded portion of the first nucleic acid molecule. In some embodiments, the one or more reagents comprises a salt. In some embodiments, the one or more reagents comprises spermine. v the one or more reagents comprises cobalt hexammine. In some embodiments, the single-stranded binding moiety comprises a single-stranded binding (SSB) protein. In some embodiments, the SSB protein is a T4 phage-derived SSB protein, an Escherichia coli-derived SSB protein, or an Extreme Thermostable SSB protein. In some embodiments, the single-stranded binding moiety comprises a third nucleic acid molecule. In some embodiments, the third nucleic acid molecule comprises a random N-mer. In some embodiments, N is between 6 and 12. In some embodiments, the third nucleic acid molecule comprises 6 bases. In some embodiments, the third nucleic acid molecule has sequence complementarity to a sequence of the single-stranded portion of the first nucleic acid molecule. In some embodiments, the single-stranded binding moiety comprises a loop structure. In some embodiments, the loop structure is included in a hairpin moiety. In some embodiments, the single-stranded binding moiety comprises a single-stranded nucleic acid molecule coupled to a moiety comprising the loop structure. In some embodiments, the composition comprises a plurality of single-stranded binding moieties comprising the single-stranded binding moiety. In some embodiments, the plurality of single-stranded binding moieties is of a same type. In some embodiments, the plurality of single-stranded binding moieties comprises multiple types of single-stranded binding moieties.

In some embodiments, at least 50% of the plurality of particles comprise immobilized thereto a nucleic acid molecule of the plurality of nucleic acid molecules that is coupled to a single-stranded binding moiety of the plurality of single-stranded binding moieties. In some embodiments, at least 70% of the plurality of particles comprise immobilized thereto a nucleic acid molecule of the plurality of nucleic acid molecules that is coupled to a single-stranded binding moiety of the plurality of single-stranded binding moieties. In some embodiments, at least 90% of the plurality of particles comprise immobilized thereto a nucleic acid molecule of the plurality of nucleic acid molecules that is coupled to a single-stranded binding moiety of the plurality of single-stranded binding moieties. In some embodiments, no more than 1% of particles of the plurality of particles are included in a particle aggregate comprising two or more particles of the plurality of particles. In some embodiments, no more than 0.1% of particles of the plurality of particles are included in a particle aggregate comprising two or more particles of the plurality of particles. In some embodiments, no more than 0.01% of particles of the plurality of particles are included in a particle aggregate comprising two or more particles of the plurality of particles. In some embodiments, particle aggregates, comprising two or more particles of the plurality of particles, having a diameter of at least 5 micrometers (µm) are absent from the composition. In some embodiments, particle aggregates, comprising two or more particles of the plurality of particles, having a diameter of at least 1 micrometers (µm) are absent from the composition. In some embodiments, particle aggregates, comprising two or more particles of the plurality of particles, having an area of at least 8 square micrometers (µm2) are absent from the composition. In some embodiments, the plurality of particles comprises a particle that does not comprise a nucleic acid molecule coupled to a single-stranded binding moiety. In some embodiments, the first particle comprises a first subset of the plurality of nucleic acid molecules immobilized thereto, wherein the first subset comprises the first nucleic acid molecule and has at least partial sequence identity to a first nucleic acid sequence. In some embodiments, the second particle comprises a second subset of the plurality of nucleic acid molecules immobilized thereto, wherein the second subset is different from the first subset, wherein the second subset has at least partial sequence identity to a second nucleic acid sequence.

In some embodiments, the second nucleic acid sequence is different than the first nucleic acid sequence. In some embodiments, the second nucleic acid sequence and the first nucleic acid sequence are identical. In some embodiments, the first subset of the plurality of nucleic acid molecules comprises at least 1,000 nucleic acid molecules. In some embodiments, the first subset of the plurality of nucleic acid molecules comprises at least 100,000 nucleic acid molecules. In some embodiments, the plurality of particles comprises at least 10,000,000 particles. In some embodiments, the plurality of particles comprises at least 1,000,000,000 particles.

Disclosed herein, are systems. In an aspect, a system comprises: (i) a first solution comprising a suspension comprising a plurality of particles comprising a first particle and a second particle, wherein the plurality of particles comprises a plurality of nucleic acid molecules immobilized thereto, wherein the first particle comprises a first subset of the plurality of nucleic acid molecules immobilized thereto, wherein the first subset comprises a first nucleic acid molecule comprising a single-stranded portion, wherein the first subset of the plurality of nucleic acid molecules has at least partial sequence identity to a first nucleic acid sequence, wherein the second particle comprises a second subset of the plurality of nucleic acid molecules immobilized thereto, wherein the second subset is different from the first subset, wherein the second subset of the plurality of nucleic acid molecules has at least partial sequence identity to a second nucleic acid sequence different from the first nucleic acid sequence; and (ii) a second solution comprising a single-stranded binding moiety configured to couple to the single-stranded portion of the first nucleic acid molecule.

In some embodiments, the plurality of particles is a plurality of beads. In some embodiments, the plurality of nucleic acid molecules comprises a plurality of deoxyribonucleic acid (DNA) molecules, ribonucleic acid (RNA) molecules, or a combination thereof. In some embodiments, the single-stranded portion comprises single-stranded deoxyribonucleic acid (ssDNA), ribonucleic acid (RNA), or a combination thereof. In some embodiments, the first nucleic acid molecule comprises a sequence of a sample nucleic acid molecule, or a complement thereof. In some embodiments, the first nucleic acid molecule comprises a priming sequence, or a complement thereof. In some embodiments, the priming sequence is a targeted priming sequence. In some embodiments, the priming sequence comprises a random N-mer sequence. In some embodiments, the first nucleic acid molecule comprises a barcode sequence or a unique molecular identifier sequence.

In some embodiments, the second solution comprises one or more reagents configured to facilitate coupling of the single-stranded binding moiety and the single-stranded portion. In some embodiments, the one or more reagents comprises a salt. In some embodiments, the one or more reagents comprises spermine. In some embodiments, the one or more reagents comprises cobalt hexammine.

In some embodiments, the single-stranded binding moiety comprises a single-stranded binding (SSB) protein. In some embodiments, the SSB protein is a T4 phage-derived SSB protein, an Escherichia coli-derived SSB protein, or an Extreme Thermostable SSB protein. In some embodiments, the single-stranded binding moiety comprises a second nucleic acid molecule. In some embodiments, the second nucleic acid molecule comprises a random N-mer. 158. In some embodiments, N is between 6 and 12. In some embodiments, the second nucleic acid molecule comprises 6 bases. In some embodiments, the second nucleic acid molecule has sequence complementarity to a sequence of the single-stranded portion of the first nucleic acid molecule. In some embodiments, the single-stranded binding moiety comprises a loop structure. In some embodiments, the loop structure is included in a hairpin moiety. In some embodiments, the single-stranded binding moiety comprises a single-stranded nucleic acid molecule coupled to a moiety comprising the loop structure. In some embodiments, the second solution comprises a plurality of single-stranded binding moieties comprising the single-stranded binding moiety. In some embodiments, the plurality of single-stranded binding moieties is of a same type. In some embodiments, the plurality of single-stranded binding moieties comprises multiple types of single-stranded binding moieties. In some embodiments, the first subset of the plurality of nucleic acid molecules comprises at least 1,000 nucleic acid molecules. In some embodiments, the first subset of the plurality of nucleic acid molecules comprises at least 100,000 nucleic acid molecules. In some embodiments, the plurality of particles comprises at least 10,000,000 particles. In some embodiments, the plurality of particles comprises at least 1,000,000,000 particles.

Disclosed herein, are methods for storing a solution comprising a plurality of particles. In an aspect, a method for storing a solution comprising a plurality of particles comprises (a) providing the solution comprising the plurality of particles, wherein the plurality of particles comprises a first set of nucleic acid molecules immobilized thereto, wherein the first set of nucleic acid molecules are configured to capture sample nucleic acid molecules derived from one or more nucleic acid samples; (b) contacting the plurality of particles with a second set of nucleic acid molecules under conditions sufficient for at least 90% of first nucleic acid molecules of the first set of nucleic acid molecules to couple to second nucleic acid molecules of the second set of nucleic acid molecules, wherein the second set of nucleic acid molecules are not the sample nucleic acid molecules; and (c) subsequent to (b), storing the solution for a time period of at least 1 hour. 172.

In some embodiments, a first particle of the plurality of particles comprises a first subset of the first set of nucleic acid molecules immobilized thereto and a second particle of the plurality of particles comprises a second subset of the first set of nucleic acid molecules immobilized thereto, wherein the second subset is different from the first subset, wherein the first subset of the first set of nucleic acid molecules have at least partial sequence identity to a first nucleic acid sequence and the second subset of the first set of nucleic acid molecules have at least partial sequence identity to a second nucleic acid sequence. In some embodiments, the first nucleic acid sequence is different from the second nucleic acid sequence. In some embodiments, the first nucleic acid sequence and the second nucleic acid sequence are identical.

In some embodiments, during storage of the solution in (c), each first nucleic acid molecule of the first set of nucleic acid molecules that is hybridized to a second nucleic acid molecule of the second set of nucleic acid molecules does not hybridize to another nucleic acid molecule of the first set of nucleic acid molecules. In some embodiments, (b) comprises contacting the plurality of particles with the second set of nucleic acid molecules under conditions sufficient for at least 95% of first nucleic acid molecules of the first set of nucleic acid molecules to couple to the second nucleic acid molecules of the second set of nucleic acid molecules.

In some embodiments, (c) comprises storing the solution at temperatures between about 18° C. to about 30° C. In some embodiments, (c) comprises storing the solution for at least 6 hours. In some embodiments, (c) comprises storing the solution for at least 24 hours. In some embodiments, (c) comprises storing the solution for at least 2 days.

In some embodiments, a second nucleic acid molecule of the second set of nucleic acid molecules comprises a sequence that is substantially complementary to a sequence of the first set of nucleic acid molecules. In some embodiments, the sequence of the first set of nucleic acid molecules comprises at least 6 bases. In some embodiments, each first nucleic acid molecule of the first set of nucleic acid molecules comprises a common nucleic acid sequence. In some embodiments, the first set of nucleic acid molecules comprises one or more different nucleic acid sequences. In some embodiments, the first set of nucleic acid molecules comprise a plurality of priming sequences. In some embodiments, the plurality of priming sequences comprises a plurality of poly(T) sequences. In some embodiments, the plurality of priming sequences comprises a plurality of random N-mer sequences. In some embodiments, the first set of nucleic acid molecules comprises a plurality of deoxyribonucleic acid (DNA) molecules, a plurality of ribonucleic acid (RNA) molecules, or a combination thereof. In some embodiments, the second set of nucleic acid molecules comprises deoxyribonucleic acid (DNA) nucleotides, ribonucleic acid (RNA) nucleotides, or a combination thereof. In some embodiments, wherein each nucleic acid molecule of the second set of nucleic acid molecules comprises at least 6 bases.

In some embodiments, the method further comprises: (d) subsequent to (c), subjecting the plurality of particles to conditions sufficient to decouple the second nucleic acid molecules of the second set of nucleic acid molecules from the first nucleic acid molecules of the first set of nucleic acid molecules. In some embodiments, (d) comprises denaturing the second nucleic acid molecules of the second set of nucleic acid molecules from the first nucleic acid molecules of the first set of nucleic acid molecules via application of a chemical or thermal stimulus. In some embodiments, the chemical stimulus comprises sodium hydroxide. In some embodiments, (d) comprises denaturing the second nucleic acid molecules of the second set of nucleic acid molecules from the first nucleic acid molecules of the first set of nucleic acid molecules via application of the thermal stimulus. In some embodiments, a first nucleic acid molecule of the first set of nucleic acid molecules hybridized to a second nucleic acid molecule of the second nucleic acid molecules has a melting point between about 35° C. and 55° C. In some embodiments, (d) comprises enzymatic degradation of the second nucleic acid molecules of the second set of nucleic acid molecules.

In some embodiments, the method further comprises, subsequent to (d), using first nucleic acid molecules of the first set of nucleic acid molecules immobilized to the plurality of particles for one or more applications selected from the group consisting of: hybridization capture of the sample nucleic acid molecules or derivatives thereof, single nucleotide polymorphism (SNP) genotyping of the sample nucleic acid molecules or derivatives thereof, sequencing library capture, synthesis of nucleic acid molecules, on-surface amplification of the sample nucleic acid molecules or derivatives thereof, and downstream processing or analysis of the sample nucleic acid molecules or derivatives thereof.

In some embodiments, the plurality of particles is immobilized to a substrate. In some embodiments, the plurality of particles is immobilized to a substantially planar array of the substrate. In some embodiments, the plurality of particles is immobilized to the substrate at independently addressable locations. In some embodiments, the independently addressable locations are substantially planar. In some embodiments, the independently addressable locations comprise one or more wells. In some embodiments, the independently addressable locations comprise one or more pillars. In some embodiments, the plurality of particles is immobilized to the substrate in a random pattern. In some embodiments, the plurality of particles is immobilized to the substrate in a predetermined pattern. In some embodiments, the plurality of particles is immobilized to the substrate with a density of at least 1,000 particles per mm².

Disclosed herein, are methods for nucleic acid processing. In an aspect, a method for nucleic acid processing comprises: (a) providing a solution comprising: (i) a plurality of particles, wherein the plurality of particles comprises a first set of nucleic acid molecules immobilized thereto, wherein the first set of nucleic acid molecules are configured to capture sample nucleic acid molecules derived from one or more nucleic acid samples; and (ii) a second set of nucleic acid molecules, wherein the second set of nucleic acid molecules are not the sample nucleic acid molecules, and wherein the second set of nucleic acid molecules comprises sequences that are substantially complementary to sequences of the first set of nucleic acid molecules, wherein the solution has been stored for a time period of at least 1 hour under conditions sufficient for at least 90% of first nucleic acid molecules of the first set of nucleic acid molecules to couple to second nucleic acid molecules of the second set of nucleic acid molecules; and (b) subjecting the plurality of particles to conditions sufficient to decouple the second nucleic acid molecules of the second set of nucleic acid molecules from the first nucleic acid molecules of the first set of nucleic acid molecules. In some embodiments, a first particle of the plurality of particles comprises a first subset of the first set of nucleic acid molecules immobilized thereto and a second particle of the plurality of particles comprises a second subset of the first set of nucleic acid molecules immobilized thereto, wherein the second subset is different from the first subset, wherein the first subset of the first set of nucleic acid molecules have at least partial sequence identity to a first nucleic acid sequence and the second subset of the first set of nucleic acid molecules have at least partial sequence identity to a second nucleic acid sequence. In some embodiments, the first nucleic acid sequence is different from the second nucleic acid sequence. In some embodiments, the first nucleic acid sequence and the second nucleic acid sequence are identical.

In some embodiments, the storage of the solution, each first nucleic acid molecule of the first set of nucleic acid molecules that is hybridized to a second nucleic acid molecule of the second set of nucleic acid molecules does not hybridize to another nucleic acid molecule of the first set of nucleic acid molecules. In some embodiments, the solution has been stored for a time period of at least 1 hour under conditions sufficient for at least 95% of first nucleic acid molecules of the first set of nucleic acid molecules to couple to second nucleic acid molecules of the second set of nucleic acid molecules. In some embodiments, prior to (b), the solution has been stored at temperatures between about 18° C. to about 30° C. In some embodiments, prior to (b), the solution has been stored for a time period of at least 6 hours. In some embodiments, prior to (b), the solution has been stored for a time period of at least 24 hours. In some embodiments, wherein, prior to (b), the solution has been stored for a time period of at least 2 days.

In some embodiments, a second nucleic acid molecule of the second set of nucleic acid molecules comprises a sequence that is substantially complementary to a sequence of the first set of nucleic acid molecules. In some embodiments, the sequence of the first set of nucleic acid molecules comprises at least 6 bases. In some embodiments, each first nucleic acid molecule of the first set of nucleic acid molecules comprises a common nucleic acid sequence. In some embodiments, the first set of nucleic acid molecules comprises one or more different nucleic acid sequences. In some embodiments, the first set of nucleic acid molecules comprises a plurality of priming sequences. In some embodiments, the plurality of priming sequences comprises a plurality of poly(T) sequences. In some embodiments, the plurality of priming sequences comprises a plurality of random N-mer sequences.

In some embodiments, the first set of nucleic acid molecules comprises a plurality of deoxyribonucleic acid (DNA) molecules, a plurality of ribonucleic acid (RNA) molecules, or a combination thereof. In some embodiments, the second set of nucleic acid molecules comprises deoxyribonucleic acid (DNA) nucleotides, ribonucleic acid (RNA) nucleotides, or a combination thereof. In some embodiments, each nucleic acid molecule of the second set of nucleic acid molecules comprises at least 6 bases.

In some embodiments, (b) comprises denaturing the second nucleic acid molecules of the second set of nucleic acid molecules from the first nucleic acid molecules of the first set of nucleic acid molecules via application of a chemical or thermal stimulus. chemical stimulus comprises sodium hydroxide. In some embodiments, (b) comprises denaturing the second nucleic acid molecules of the second set of nucleic acid molecules from the first nucleic acid molecules of the first set of nucleic acid molecules via application of the thermal stimulus. In some embodiments, a first nucleic acid molecule of the first set of nucleic acid molecules hybridized to a second nucleic acid molecule of the second nucleic acid molecules has a melting point between about 35° C. and 55° C. In some embodiments, (b) comprises enzymatic degradation of the second nucleic acid molecules of the second set of nucleic acid molecules.

In some embodiments, the method comprises, subsequent to (b), using the first set of nucleic acid molecules immobilized to a surface for one or more applications selected from the group consisting of: hybridization capture of the sample nucleic acid molecules or derivatives thereof, single nucleotide polymorphism (SNP) genotyping of the sample nucleic acid molecules or derivatives thereof, sequencing library capture, synthesis of nucleic acid molecules, on-surface amplification of the sample nucleic acid molecules or derivatives thereof, and downstream processing or analysis of the sample nucleic acid molecules or derivatives thereof.

In some embodiments, the plurality of particles is immobilized to a substrate. In some embodiments, the plurality of particles is immobilized to a substantially planar array of the substrate. In some embodiments, the plurality of particles is immobilized to the substrate at independently addressable locations. In some embodiments, the independently addressable locations are substantially planar. In some embodiments, the independently addressable locations comprise one or more wells. In some embodiments, the independently addressable locations comprise one or more pillars. In some embodiments, the plurality of particles is immobilized to the substrate in a random pattern. In some embodiments, the plurality of particles is immobilized to the substrate in a predetermined pattern. In some embodiments, the plurality of particles is immobilized to the substrate with a density of at least 1,000 molecules per mm².

Disclosed herein, are kits. In an aspect, a kit, comprising: (i) a first solution comprising a plurality of particles comprising a first set of nucleic acid molecules immobilized thereto, wherein the first set of nucleic acid molecules are configured to capture sample nucleic acid molecules derived from one or more nucleic acid samples; and (ii) a second set of nucleic acid molecules, wherein the second set of nucleic acid molecules comprises one or more second nucleic acid molecules, which one or more second nucleic acid molecules are not the sample nucleic acid molecules, and wherein the second set of nucleic acid molecules comprises sequences that are substantially complementary to sequences of the first set of nucleic acid molecules such that, upon contacting the plurality of particles with the second set of nucleic acid molecules, at least 70% of first nucleic acid molecules of the first set of nucleic acid molecules couple to second nucleic acid molecules of the second set of nucleic acid molecules.

In some embodiments, a first particle of the plurality of particles comprises a first subset of the first set of nucleic acid molecules immobilized thereto, and wherein a second particle of the plurality of particles comprises a second subset of the first set of nucleic acid molecules immobilized thereto, wherein the second subset is different from the first subset, wherein the first subset of the first set of nucleic acid molecules have at least partial identity to a first nucleic acid sequence and the second subset of the first set of nucleic acid molecules have at least partial sequence identity to a second nucleic acid sequence. In some embodiments, the first nucleic acid sequence is different from the second nucleic acid sequence. In some embodiments, the first nucleic acid sequence and the second nucleic acid sequence are identical. In some embodiments, the second set of nucleic acid molecules are included in a second solution separate from the first solution. In some embodiments, the second set of nucleic acid molecules are included in the second solution.

In some embodiments, a second nucleic acid molecule of the second set of nucleic acid molecules comprises a sequence that is substantially complementary to a sequence of the first set of nucleic acid molecules. In some embodiments, the sequence of the first set of nucleic acid molecules comprises at least 6 bases. In some embodiments, each first nucleic acid molecule of the first set of nucleic acid molecules comprises a common nucleic acid sequence. In some embodiments, the first set of nucleic acid molecules comprises one or more different nucleic acid sequences. In some embodiments, the first set of nucleic acid molecules comprises a plurality of priming sequences. In some embodiments, the plurality of priming sequences comprises a plurality of poly(T) sequences. In some embodiments, the plurality of priming sequences comprises a plurality of random N-mer sequences. In some embodiments, the first set of nucleic acid molecules comprises a plurality of deoxyribonucleic acid (DNA) molecules, a plurality of ribonucleic acid (RNA) molecules, or a combination thereof. In some embodiments, the second set of nucleic acid molecules comprises deoxyribonucleic acid (DNA) nucleotides, ribonucleic acid (RNA) nucleotides, or a combination thereof. In some embodiments, each nucleic acid molecule of the second set of nucleic acid molecules comprises at least 6 bases. In some embodiments, the sequences of the first set of nucleic acid molecules comprise between about 6-20 bases.

In some embodiments, the sequences of the first set of nucleic acid molecules and the sequences of the second set of nucleic acid molecules have the same number of nucleotides. In some embodiments, the sequences of the first set of nucleic acid molecules and the sequences of the second set of nucleic acid molecules have different numbers of nucleotides. In some embodiments, the sequences of the first set of nucleic acid molecules comprises one or more different nucleic acid sequences. In some embodiments, the sequences of the first set of nucleic acid molecules are identical.

In some embodiments, the method further comprises a chemical stimulus configured to decouple the first nucleic acid molecules from the second nucleic acid molecules. In some embodiments, the chemical stimulus comprises sodium hydroxide.

In some embodiments, the plurality of particles is immobilized to a substrate. In some embodiments, the plurality of particles is immobilized to a substantially planar array of the substrate. In some embodiments, the plurality of particles is immobilized to the substrate at independently addressable locations. In some embodiments, the independently addressable locations are substantially planar. In some embodiments, the independently addressable locations comprise one or more wells. In some embodiments, the independently addressable locations comprise one or more pillars. In some embodiments, the plurality of particles is immobilized to the substrate in a random pattern. In some embodiments, the plurality of particles is immobilized to the substrate in a predetermined pattern. In some embodiments, the plurality of particles is immobilized to the substrate with a density of at least 1,000 particles per mm².

Disclosed herein, are methods for dispensing a plurality of particles onto a substrate. In an aspect, a method for dispensing a plurality of particles onto a substrate comprises: (a) incubating the plurality of particles with a first buffer solution, wherein the first buffer solution is substantially depleted of a cation, wherein each of at least a subset of the plurality of particles comprises a nucleic acid molecule immobilized thereto; (b) loading the substrate with a second buffer solution, wherein the second buffer solution comprises the cation; and (c) dispensing the plurality of particles onto the substrate to immobilize at least the subset of the plurality of particles onto a plurality of individually addressable locations on the substrate

In some embodiments, the cation comprises a divalent cation. In some embodiments, the divalent cation comprises a magnesium ion or a calcium ion. In some embodiments, the divalent cation comprises the magnesium ion. In some embodiments, the magnesium ion comprises Mg²⁺. In some embodiments, the divalent cation comprises the calcium ion. In some embodiments, the calcium ion comprises Ca²⁺. In some embodiments, the first buffer solution is free of the cation. In some embodiments, the second buffer solution comprises at least about 5 millimolar (mM) of the cation. In some embodiments, the second buffer solution comprises at least about 10 millimolar (mM) of the cation. In some embodiments, the second buffer solution comprises at least about 25 millimolar (mM) of the cation. In some embodiments, the second buffer solution comprises at least about 50 millimolar (mM) of the cation.

In some embodiments, the first buffer solution comprises a Tris buffer solution. In some embodiments, the Tris buffer solution comprises about 10 (millimolar) mM of Tris. In some embodiments, the Tris buffer solution has a pH of about 7.0. In some embodiments, the second buffer solution comprises a Tris buffer solution. In some embodiments, the Tris buffer solution comprises about 10 (millimolar) mM of Tris. In some embodiments, the Tris buffer solution has a pH of about 7.0. In some embodiments, the Tris buffer solution comprises about 0.055% Tergitol by volume.

In some embodiments, the plurality of particles comprises at least about 100,000 particles. In some embodiments, the plurality of particles comprises at least about 10,000,000 particles. In some embodiments, the plurality of particles comprises at least about 1,000,000,000 particles. In some embodiments, subsequent to (c), at least 100,000 particles are immobilized on the substrate. In some embodiments, subsequent to (c), at least 1,000,000,000 particles are immobilized on the substrate. In some embodiments, the plurality of particles, subsequent to contacting the first buffer solution, comprises a concentration of at least about 100,000 particles per microliter (µL) in the first buffer solution. In some embodiments, the plurality of particles, subsequent to contacting the first buffer solution, comprises a concentration of at least about 1,000,000 particles per microliter (µL) in the first buffer solution. In some embodiments, the plurality of particles, subsequent to contacting the first buffer solution, comprises a concentration of at least about 20,000,000 particles per microliter (µL) in the first buffer solution. In some embodiments, the first buffer solution has a volume of less than about 1 milliliter (mL). In some embodiments, the first buffer solution has a volume of less than about 100 microliter (µL). In some embodiments, the first buffer solution has a volume of less than about 20 microliter (µL).

In some embodiments, (a) comprises a first incubation time of at least about 0.5 minutes. In some embodiments, the method further comprises, subsequent to (b), incubating the substrate with the second buffer solution. In some embodiments, the incubating the substrate with the second buffer solution comprises a second incubation time of at least about 60 minutes. In some embodiments, the method further comprises, subsequent to (b), forming a layer of the cation on the substrate. In some embodiments, the layer has a thickness of at least about 10-20 micrometers (µm).

In some embodiments, the plurality of individually addressable locations comprises at least about 100,000 locations. In some embodiments, at least about 60% of the plurality of independently addressable locations has at least one of the plurality of particles immobilized thereto. In some embodiments, at least about 90% of the plurality of independently addressable locations has at least one of the plurality of particles immobilized thereto.

In some embodiments, the center of each independently addressable location of the plurality of individually addressable locations is separated by fewer than about 10 micrometers (µm). In some embodiments, the center of each independently addressable location of the plurality of individually addressable locations is separated by fewer than about 5 micrometers (µm). In some embodiments, the center of each independently addressable location of the plurality of individually addressable locations is separated by about 1.8 micrometers (µm). In some embodiments, the center of each independently addressable location of the plurality of individually addressable locations is separated by about 1.5 micrometers (µm). In some embodiments, the center of each independently addressable location of the plurality of individually addressable locations is separated by about 1 micrometers (µm). In some embodiments, the plurality of independently addressable locations is substantially planar. In some embodiments, the plurality of independently addressable locations comprises one or more wells. In some embodiments, the plurality of independently addressable locations comprises one or more pillars. In some embodiments, the plurality of particles is a plurality of beads. In some embodiments, an average maximum dimension of the plurality of particles shrinks upon contacting the second buffer solution. In some embodiments, the average maximum dimension of the plurality of particles shrinks by at least 5% upon contacting the second buffer solution. In some embodiments, the nucleic acid molecule comprises deoxyribonucleic acid (DNA) nucleotides, ribonucleic acid (RNA) nucleotides, or a combination thereof. In some embodiments, the nucleic acid molecule comprises deoxyribonucleic acid (DNA) nucleotides. In some embodiments, the nucleic acid molecule comprises ribonucleic acid (RNA) nucleotides.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a schematic illustration of a particle comprising a nucleic acid molecule immobilized thereto.

FIG. 2A shows an image taken of a substrate loaded with particles comprising human genomic deoxyribonucleic acid (DNA) following titration thereon in the absence of a single-strand binding protein. FIG. 2B shows an image taken of a substrate loaded with particles comprising human genomic DNA following titration thereon in the presence of a T4 phage-derived single-strand binding protein.

FIG. 3A shows another image taken of a substrate loaded with particles comprising human genomic DNA following titration thereon in the absence of a single-strand binding protein. FIGS. 3B-3D show images taken of substrates loaded with particles comprising human genomic DNA following titration thereon in the presence of varying amounts of an Extreme Thermostable (ET) single-strand binding protein (FIG. 3B: 40 ng SSB/million particles; FIG. 3C: 60 ng SSB/million particles; and FIG. 3D: 80 ng SSB/million beads).

FIG. 4A shows another image taken following titration of particles comprising human genomic DNA in the absence of a single-strand binding protein. FIG. 4B shows an image taken following titration of particles comprising human genomic DNA in the presence of a E. coli-derived single-strand binding protein.

FIGS. 5A and 5B show relative coverage in sequencing assays performed in the absence (FIG. 5A) and presence (FIG. 5B) of single-strand binding proteins.

FIGS. 6A-6C show flow cytometry signals measured for particle populations in the absence of single-strand binding proteins (FIG. 6A), in the presence of single-strand binding proteins (FIG. 6B), and in the presence of random hexamers (FIG. 6C).

FIGS. 7A and 7B show relative coverage in sequencing assays performed in the presence (FIG. 7A) and absence (FIG. 7B) of single-stranded binding proteins.

FIG. 8A demonstrates formation of a particle aggregate comprising two particles (upper panel) and use of a blocking nucleic acid molecule to prevent formation of a particle aggregate (lower panel). FIG. 8B shows a particle comprising nucleic acid molecules blocked with complementary nucleic acid molecules.

FIGS. 9A and 9B show images taken of a substrate loaded with particles comprising single-stranded human genomic deoxyribonucleic acid (DNA) (FIG. 9A) and double-stranded human genomic DNA (FIG. 9B).

FIG. 10 show histograms showing flow cytometric analysis of particles comprising single-stranded nucleic acid molecules and treated with varying concentrations of complementary oligomers.

FIGS. 11A and 11B show a scheme for stabilizing a molecule hybridized to a particle. FIG. 11C shows a comparison of double-stranded molecules and double-stranded molecules including a loop feature.

FIGS. 12A-12G illustrate different examples of cross-sectional surface profiles of a substrate.

FIGS. 13A-13B illustrate methods for loading beads onto a substrate. FIG. 13A illustrates a method for loading beads onto specific regions of a substrate. FIG. 13B illustrates a method for loading a subset of beads onto specific regions of a substrate.

FIG. 14 shows an example coating of a substrate with a hexagonal lattice of beads.

FIG. 15A shows an example system and method for loading a sample or a reagent onto a substrate. FIG. 15B shows another example system and method for loading a sample or a reagent onto a substrate.

FIG. 16 shows a flowchart for an example of a method for sequencing a nucleic acid molecule.

FIG. 17 illustrates example individually addressable locations on different substrates.

FIGS. 18A-18D show representative images of bead aggregation on a wafer coupon upon loading onto the wafer after the beads were incubated for different amounts of time. Circles highlight selected bead aggregates that were detected. * indicates selected regions with significant aggregation. * * indicates selected regions of poor bead loading.

FIGS. 19A-19E show representative images of bead aggregation on a wafer coupon with a pitch size of 1.8 µm. The wafer surfaces were incubated in a buffer with different levels of Mg²⁺ (prewet) and the beads were incubated in a buffer with different levels of Mg²⁺ (load) before the beads were loaded onto the wafer surface. Circles highlight selected bead aggregates that were detected.

FIGS. 20A-20B show representative images of bead aggregation on a wafer coupon with a pitch size of 1.5 µm. The wafer surfaces were incubated in a buffer with different levels of Mg²⁺ (prewet) and the beads were incubated in a buffer with different levels of Mg²⁺ (load) before the beads were loaded onto the wafer surface. Circles highlight example bead aggregates.

FIGS. 21A-21E show representative images of bead aggregation on a wafer coupon with a pitch size of 1 × 1.5 µm. The wafer surfaces were incubated in a buffer with different levels of Mg²⁺ (prewet) and the beads (amplified IH beads, B1434 (10%-tBA ATRP in THF), PA39FAM labeled) were incubated in a buffer with different levels of Mg²⁺ (load) before the beads were loaded onto the wafer surface. Circles highlight example bead aggregates that were detected.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Ranges may be expressed herein as from “about” one particular value, and/or to “about” another particular value. The terms “about” and “approximately” shall generally mean an acceptable degree of error or variation for a given value or range of values, such as, for example, a degree of error or variation that is within 20 percent (%), within 15%, within 10%, or within 5% of a given value or range of values.

The term “subject,” as used herein, generally refers to an individual or entity from which a biological sample (e.g., a biological sample that is undergoing or will undergo processing or analysis) may be derived. A subject may be an animal (e.g., mammal or non-mammal) or plant. The subject may be a human, dog, cat, horse, pig, bird, non-human primate, simian, farm animal, companion animal, sport animal, or rodent. A subject may be a patient. The subject may have or be suspected of having a disease or disorder, such as cancer (e.g., breast cancer, colorectal cancer, brain cancer, leukemia, lung cancer, skin cancer, liver cancer, pancreatic cancer, lymphoma, esophageal cancer or cervical cancer) or an infectious disease. Alternatively or additionally, a subject may be known to have previously had a disease or disorder. The subject may have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn’s disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington’s disease, Klinefelter syndrome, Marfan syndrome, myotonic dystrophy, neurofibromatosis, Noonan syndrome, osteogenesis imperfecta, Parkinson’s disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease. A subject may be undergoing treatment for a disease or disorder. A subject may be symptomatic or asymptomatic of a given disease or disorder. A subject may be healthy (e.g., not suspected of having disease or disorder). A subject may have one or more risk factors for a given disease. A subject may be under the care of one or more health professionals. The subject may be undergoing treatment. The subject may not be undergoing treatment. A subject may have a given weight, height, body mass index, or other physical characteristics. A subject may have a given ethnic or racial heritage, place of birth or residence, nationality, disease or remission state, family medical history, or other characteristics.

As used herein, the term “biological sample” generally refers to any sample obtained from a subject or specimen. The biological sample can be a fluid or tissue from the subject or specimen. The fluid can be blood (e.g., whole blood), saliva, urine, or sweat. The tissue can be from an organ (e.g., liver, lung, or thyroid), or a mass of cellular material, such as, for example, a tumor. The biological sample can be a feces sample, collection of cells (e.g., cheek swab), or hair sample. The biological sample can be a cell-free or cellular sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid molecules may be cell-free or cell-free nucleic acid molecules, such as cell free DNA or cell free RNA. The nucleic acid molecules may be derived from a variety of sources. The biological sample may be obtained directly or indirectly from the subject. A sample may be obtained from a subject via any suitable method, including, but not limited to, spitting, swabbing, blood draw, biopsy, obtaining excretions (e.g., urine, stool, sputum, vomit, or saliva), excision, scraping, and puncture. A sample may be obtained from a subject by, for example, intravenously or intraarterially accessing the circulatory system, collecting a secreted biological sample (e.g., stool, urine, saliva, sputum, etc.), breathing, or surgically extracting a tissue (e.g., biopsy). The sample may be obtained by non-invasive methods including but not limited to: scraping of the skin or cervix, swabbing of the cheek, or collection of saliva, urine, feces, menses, tears, or semen. Alternatively, the sample may be obtained by an invasive procedure such as biopsy, needle aspiration, or phlebotomy. A sample may comprise a bodily fluid such as, but not limited to, blood (e.g., whole blood, red blood cells, leukocytes or white blood cells, platelets), plasma, serum, sweat, tears, saliva, sputum, urine, semen, mucus, synovial fluid, breast milk, colostrum, amniotic fluid, bile, bone marrow, interstitial or extracellular fluid, or cerebrospinal fluid. For example, a sample may be obtained by a puncture method to obtain a bodily fluid comprising blood and/or plasma. Such a sample may comprise both cells and cell-free nucleic acid material. Alternatively, the sample may be obtained from any other source including but not limited to blood, sweat, hair follicle, buccal tissue, tears, menses, feces, or saliva. The biological sample may be a tissue sample, such as a tumor biopsy. The sample may be obtained from any of the tissues provided herein including, but not limited to, skin, heart, lung, kidney, breast, pancreas, liver, intestine, brain, prostate, esophagus, muscle, smooth muscle, bladder, gall bladder, colon, or thyroid. The methods of obtaining provided herein include methods of biopsy including fine needle aspiration, core needle biopsy, vacuum assisted biopsy, large core biopsy, incisional biopsy, excisional biopsy, punch biopsy, shave biopsy or skin biopsy. The biological sample may comprise one or more cells. A biological sample may comprise one or more nucleic acid molecules such as one or more deoxyribonucleic acid (DNA) and/or ribonucleic acid (RNA) molecules (e.g., included within cells or not included within cells). Nucleic acid molecules may be included within cells. Alternatively or additionally, nucleic acid molecules may not be included within cells (e.g., cell-free nucleic acid molecules). The biological sample may be a cell-free sample.

The term “cell-free sample,” as used herein, generally refers to a sample that is substantially free of cells (e.g., less than 10% cells on a volume basis). A cell-free sample may be derived from any source (e.g., of a subject as described herein). For example, a cell-free sample may be derived from blood, sweat, urine, or saliva. For example, a sample from a first tissue or fluid may be combined with a sample from a second tissue or fluid (e.g., while the samples are obtained or after the samples are obtained). In an example, a first fluid and a second fluid may be collected from a subject (e.g., at the same or different times) and the first and second fluids may be combined to provide a sample. A cell-free sample may comprise one or more nucleic acid molecules such as one or more DNA or RNA molecules.

A sample that is not a cell-free sample (e.g., a sample comprising one or more cells) may be processed to provide a cell-free sample. For example, a sample that includes one or more cells as well as one or more nucleic acid molecules (e.g., DNA and/or RNA molecules) not included within cells (e.g., cell-free nucleic acid molecules) may be obtained from a subject. The sample may be subjected to processing (e.g., as described herein) to separate cells and other materials from the nucleic acid molecules not included within cells, thereby providing a cell-free sample (e.g., comprising nucleic acid molecules not included within cells). The cell-free sample may then be subjected to further analysis and processing (e.g., as provided herein). Nucleic acid molecules not included within cells (e.g., cell-free nucleic acid molecules) may be derived from cells and tissues. For example, cell-free nucleic acid molecules may derive from a tumor tissue or a degraded cell (e.g., of a tissue of a body). Cell-free nucleic acid molecules may comprise any type of nucleic acid molecules (e.g., as described herein). Cell-free nucleic acid molecules may be double-stranded, single-stranded, or a combination thereof. Cell-free nucleic acid molecules may be released into a bodily fluid through secretion or cell death processes, e.g., cellular necrosis, apoptosis, or the like. Cell-free nucleic acid molecules may be released into bodily fluids from cancer cells (e.g., circulating tumor DNA (ctDNA)). Cell free nucleic acid molecules may also be fetal DNA circulating freely in a maternal blood stream (e.g., cell-free fetal nucleic acid molecules such as cffDNA). Alternatively or additionally, cell-free nucleic acid molecules may be released into bodily fluids from healthy cells.

A biological sample may be obtained directly from a subject and analyzed without any intervening processing, such as, for example, sample purification or extraction. For example, a blood sample may be obtained directly from a subject by accessing the subject’s circulatory system, removing the blood from the subject (e.g., via a needle), and transferring the removed blood into a receptacle. The receptacle may comprise reagents (e.g., anti-coagulants) such that the blood sample is useful for further analysis. Such reagents may be used to process the sample or analytes derived from the sample in the receptacle or another receptacle prior to analysis. In another example, a swab may be used to access epithelial cells on an oropharyngeal surface of the subject. Following obtaining the biological sample from the subject, the swab containing the biological sample may be contacted with a fluid (e.g., a buffer) to collect the biological fluid from the swab.

A sample (e.g., a biological sample or cell-free biological sample) may undergo one or more processes in preparation for analysis, including, but not limited to, filtration, centrifugation, selective precipitation, permeabilization, isolation, agitation, heating, purification, and/or other processes. For example, a sample may be filtered to remove contaminants or other materials. In an example, a sample comprising cells may be processed to separate the cells from other material in the sample. Such a process may be used to prepare a sample comprising only cell-free nucleic acid molecules. Such a process may consist of a multi-step centrifugation process. Multiple samples, such as multiple samples from the same subject (e.g., obtained in the same or different manners from the same or different bodily locations, and/or obtained at the same or different times (e.g., seconds, minutes, hours, days, weeks, months, or years apart)) or multiple samples from different subjects may be obtained for analysis as described herein. In an example, the first sample is obtained from a subject before the subject undergoes a treatment regimen or procedure and the second sample is obtained from the subject after the subject undergoes the treatment regimen or procedure. Alternatively or additionally, multiple samples may be obtained from the same subject at the same or approximately the same time. Different samples obtained from the same subject may be obtained in the same or different manner. For example, a first sample may be obtained via a biopsy and a second sample may be obtained via a blood draw. Samples obtained in different manners may be obtained by different medical professionals, using different techniques, at different times, and/or at different locations. Different samples obtained from the same subject may be obtained from different areas of a body (e.g., different tissues).

A biological sample as described herein may contain a target nucleic acid and/or a plurality of nucleic acid molecules. For example, a biological sample may comprise a plurality of target nucleic acid molecules from a single subject. In another example, a biological sample may comprise a first target nucleic acid molecule from a first subject and a second target nucleic acid molecule from a second subject. As used herein, the terms “template nucleic acid”, “target nucleic acid”, “nucleic acid molecule,” “nucleic acid sequence,” “nucleic acid fragment,” “oligonucleotide,” “polynucleotide,” and “nucleic acid” generally refer to polymeric forms of nucleotides of any length, such as deoxyribonucleotides (dNTPs) or ribonucleotides (rNTPs), or analogs thereof, and may be used interchangeably. Nucleic acids may have any three-dimensional structure, and may perform any function, known or unknown. A nucleic acid molecule may have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3 kb, 4 kb, 5 kb, 10 kb, 50 kb, or more. An oligonucleotide is typically composed of a specific sequence of four nucleotide bases: adenine (A); cytosine (C); guanine (G); and thymine (T) (uracil (U) for thymine (T) when the polynucleotide is RNA). Oligonucleotides may include one or more nonstandard nucleotide(s), nucleotide analog(s) and/or modified nucleotides. Non-limiting examples of nucleic acids include DNA, RNA, genomic DNA (e.g., gDNA such as sheared gDNA), cell-free DNA (e.g., cfDNA), synthetic DNA/RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, complementary DNA (cDNA), recombinant nucleic acids, branched nucleic acids, plasmids, vectors, isolated DNA of any sequence, isolated RNA of any sequence, nucleic acid probes, and primers. A nucleic acid may comprise one or more nonstandard nucleotide(s), nucleotide analog(s) and/or modified nucleotides (e.g., methylated nucleotides). If present, modifications to the nucleotide structure may be made before or following assembly of the nucleic acid. The sequence of nucleotides of a nucleic acid may be interrupted by non-nucleotide components. A nucleic acid may be further modified following polymerization, such as by conjugation or binding with a reporter agent.

A target nucleic acid or sample nucleic acid as described herein may be amplified to generate an amplified product. A target nucleic acid may be a target RNA or a target DNA. When the target nucleic acid is a target RNA, the target RNA may be any type of RNA, including types of RNA described elsewhere herein. The target RNA may be viral RNA and/or tumor RNA. A viral RNA may be pathogenic to a subject. Non-limiting examples of pathogenic viral RNA include human immunodeficiency virus I (HIV I), human immunodeficiency virus II (HIV II), orthomyxoviruses, Ebola virus, Dengue virus, influenza viruses (e.g., H1N1, H3N2, H7N9, or H5N1), herpesvirus, hepatitis A virus, hepatitis B virus, hepatitis C virus (e.g., armored RNA-HCV virus), hepatitis D virus, hepatitis E virus, hepatitis G virus, coronaviruses, Epstein-Barr virus, mononucleosis virus, cytomegalovirus, SARS virus, West Nile Fever virus, polio virus, and measles virus.

The term “nucleotide,” as used herein, generally refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a modified or engineered nucleotide).

The term “nucleotide analog,” as used herein, may include, but is not limited to, a nucleotide that may or may not be a naturally occurring nucleotide. For example, a nucleotide analog may be derived from and/or include structural similarities to a canonical nucleotide such as adenine- (A), thymine- (T), cytosine- (C), uracil- (U), or guanine- (G) including nucleotide. A nucleotide analog may comprise one or more differences or modifications relative to a natural nucleotide. Examples of nucleotide analogs include inosine, diaminopurine, 5-fluorouracil, 5-bromouracil, 5-chlorouracil, 5-iodouracil, hypoxanthine, xanthine, deazaxanthine, deazaguanine, isocytosine, isoguanine, 4- acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids, and modified versions thereof (e.g., by oxidation, reduction, and/or addition of a substituent such as an alkyl, hydroxyalkyl, hydroxyl, or halogen moiety). Nucleic acid molecules (e.g., polynucleotides, double-stranded nucleic acid molecules, single-stranded nucleic acid molecules, primers, adapters, etc.) may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. In some cases, a nucleotide may include a modification in its phosphate moiety, including a modification to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates), and modifications with selenium moieties (e.g., phosphoroselenoate nucleic acids). A nucleotide or nucleotide analog may comprise a sugar selected from the group consisting of ribose, deoxyribose, and modified versions thereof (e.g., by oxidation, reduction, and/or addition of a substituent such as an alkyl, hydroxyalkyl, hydroxyl, or halogen moiety). A nucleotide analog may also comprise a modified linker moiety (e.g., in lieu of a phosphate moiety). Nucleotide analogs may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure may provide, for example, higher density in bits per cubic mm, higher safety (resistant to accidental or purposeful synthesis of natural toxins), easier discrimination in photo-programmed polymerases, and/or lower secondary structure. Nucleotide analogs may be capable of reacting or bonding with detectable moieties for nucleotide detection.

The term “amplification,” as used herein, generally refers to the production of one or more copies of a nucleic acid molecule or an extension product (e.g., a product of a primer extension reaction on the nucleic acid molecule). Amplification of a nucleic acid molecule may yield a single strand hybridized to the nucleic acid molecule, or multiple copies of the nucleic acid molecule or complement thereof. An amplicon may be a single-stranded or double-stranded nucleic acid molecule that is generated by an amplification procedure from a starting template nucleic acid molecule. The amplicon may comprise a nucleic acid strand, of which at least a portion may be substantially identical or substantially complementary to at least a portion of the starting template. Where the starting template is a double-stranded nucleic acid molecule, an amplicon may comprise a nucleic acid strand that is substantially identical to at least a portion of one strand and is substantially complementary to at least a portion of either strand. The amplicon can be single-stranded or double-stranded irrespective of whether the initial template is single-stranded or double-stranded. An amplification reaction may be, for example, a polymerase chain reaction (PCR), such as an emulsion polymerase chain reaction (ePCR; e.g., PCR carried out within a microreactor such as a well or droplet).

The term “clonal,” as used herein, generally refers to a population of nucleic acids for which a substantial portion (e.g., greater than 50%, 60%, 70%, 80%, 90%, 95%, or 99%) of its members have substantially identical sequences. Members of a clonal population of nucleic acid molecules may have sequence homology to one another. In some instances, such members may have sequence homology to a template nucleic acid molecule. In some instances, such members may have sequence homology to a complement of the template nucleic acid molecule (if single stranded). The members of the clonal population may be double stranded or single stranded. Members of a population may not be 100% identical or complementary because, e.g., “errors” may occur during the course of synthesis such that a minority of a given population may not have sequence homology with a majority of the population. For example, at least 50% of the members of a population may be substantially identical to each other or to a reference nucleic acid molecule (i.e., a molecule of defined sequence used as a basis for a sequence comparison). At least 60%, at least 70%, at least 80%, at least 90%, at least 95%, at least 99%, or more of the members of a population may be substantially identical to the reference nucleic acid molecule. Two molecules may be considered substantially identical (or homologous) if the percent identity between the two molecules is at least 60%, 70%, 75%, 80%, 85%, 90%, 95%, 98%, 99%, 99.9% or greater. Two molecules may be considered substantially complementary if the percent complementarity between the two molecules is at least 60%, 70%, 75%, 80%, 85%, 90%, 95%, 98%, 99%, 99.9% or greater. A low or insubstantial level of mixing of non-homologous nucleic acids may occur, and thus a clonal population may contain a minority of diverse nucleic acids (e.g., less than 30%, e.g., less than 10%).

The term “polymerizing enzyme” or “polymerase,” as used herein, generally refers to a substance catalyzing a polymerization reaction. A polymerizing enzyme may be used to extend a nucleic acid primer paired with a template strand by incorporation of nucleotides or nucleotide analogs. A polymerizing enzyme may add a new strand of DNA by extending the 3′ end of an existing nucleotide chain, adding new nucleotides matched to the template strand one at a time via the creation of phosphodiester bonds. A polymerizing enzyme may be a polymerase such as a nucleic acid polymerase. A polymerase may be naturally occurring or synthesized. A polymerase may have relatively high processivity, namely the capability of the polymerase to consecutively incorporate nucleotides into a nucleic acid template without releasing the nucleic acid template. A polymerizing enzyme may be a transcriptase. Examples of polymerases include, but are not limited to, a DNA polymerase, an RNA polymerase, a thermostable polymerase, a wild-type polymerase, a modified polymerase, E. coli DNA polymerase I, T7 DNA polymerase, bacteriophage T4 DNA polymerase, 029 (phi29) DNA polymerase, Taq polymerase, Tth polymerase, Tli polymerase, Pfu polymerase, Pwo polymerase, VENT polymerase, DEEPVENT polymerase, EXTaq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tea polymerase, Tih polymerase, Tfi polymerase, Platinum Taq polymerases, Tbr polymerase, Tfl polymerase, Pfutubo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. A polymerase may be a single subunit polymerase.

The term “complementary sequence,” as used herein, generally refers to a sequence that hybridizes to another sequence or has sequence complementarity with such other sequence. Hybridization between two single-stranded nucleic acid molecules may involve the formation of a double-stranded structure that is stable under certain conditions. Two single-stranded polynucleotides may be considered to be hybridized if they are bonded to each other by two or more sequentially adjacent base pairings. A substantial proportion of nucleotides in one strand of a double-stranded structure may undergo Watson-Crick base-pairing with a nucleoside on the other strand. Hybridization may also include the pairing of nucleoside analogs, such as deoxyinosine, nucleosides with 2-aminopurine bases, and the like, that may be employed to reduce the degeneracy of probes, whether or not such pairing involves formation of hydrogen bonds.

The term “denaturation,” as used herein, generally refers to separation of a double-stranded molecule (e.g., DNA) into single-stranded molecules. Denaturation may be complete or partial denaturation. In partial denaturation, a single-stranded region may form in a double-stranded molecule by denaturation of the two deoxyribonucleic acid (DNA) strands flanked by double-stranded regions in DNA.

The term “melting temperature” or “melting point,” as used herein, generally refers to the temperature at which at least a portion of a strand of a nucleic acid molecule in a sample has separated from at least a portion of a complementary strand. The melting temperature may be the temperature at which a double-stranded nucleic acid molecule has partially or completely denatured. The melting temperature may refer to a temperature of a sequence among a plurality of sequences of a given nucleic acid molecule, or a temperature of the plurality of sequences. Different regions of a double-stranded nucleic acid molecule may have different melting temperatures. For example, a double-stranded nucleic acid molecule may include a first region having a first melting point and a second region having a second melting point that is higher than the first melting point. Accordingly, different regions of a double-stranded nucleic acid molecule may melt (e.g., partially denature) at different temperatures. The melting point of a nucleic acid molecule or a region thereof (e.g., a nucleic acid sequence) may be determined experimentally (e.g., via a melt analysis or other procedure) or may be estimated based upon the sequence and length of the nucleic acid molecule. For example, a software program such as MELTING may be used to estimate a melting temperature for a nucleic acid sequence (Dumousseau M, Rodriguez N, Juty N, Le Novère N, MELTING, a flexible platform to predict the melting temperatures of nucleic acids. BMC Bioinformatics. 2012 May 16;13:101. doi: 10.1186/1471-2105-13-101). Accordingly, a melting point as described herein may be an estimated melting point. A true melting point of a nucleic acid sequence may vary based upon the sequences or lack thereof adjacent to the nucleic acid sequence of interest as well as other factors.

The term “sequencing,” as used herein, generally refers to a process for generating or identifying a sequence of a biological molecule, such as a nucleic acid molecule. Such sequence may be a nucleic acid sequence, which may include a sequence of nucleic acid bases (e.g., nucleobases). Sequencing may be, for example, single molecule sequencing, sequencing by synthesis, sequencing by hybridization, or sequencing by ligation. Sequencing may be performed using template nucleic acid molecules immobilized on a support, such as a flow cell or one or more beads. A sequencing assay may yield one or more sequencing reads corresponding to one or more template nucleic acid molecules.

The term “read,” as used herein, generally refers to a nucleic acid sequence, such as a sequencing read. A sequencing read may be an inferred sequence of nucleic acid bases (e.g., nucleotides) or base pairs obtained via a nucleic acid sequencing assay. A sequencing read may be generated by a nucleic acid sequencer, such as a massively parallel array sequencer (e.g., Illumina or Pacific Biosciences of California). A sequencing read may correspond to a portion, or in some cases all, of a genome of a subject. A sequencing read may be part of a collection of sequencing reads, which may be combined through, for example, alignment (e.g., to a reference genome), to yield a sequence of a genome of a subject.

The term “support” or “substrate,” as used herein, generally refers to any solid or semi-solid article on which reagents such as nucleic acid molecules may be immobilized. Nucleic acid molecules may be synthesized, attached, ligated, or otherwise immobilized. Nucleic acid molecules may be immobilized on a substrate by any method including, but not limited to, physical adsorption, by ionic or covalent bond formation, or combinations thereof. A substrate may be 2-dimensional (e.g., a planar 2D substrate) or 3-dimensional. In some cases, a substrate may be a component of a flow cell and/or may be included within or adapted to be received by a sequencing instrument. A substrate may include a polymer, a glass, or a metallic material. Examples of substrates include a membrane, a planar substrate, a microtiter plate, a bead (e.g., a magnetic bead), a filter, a test strip, a slide, a cover slip, and a test tube. A substrate may comprise organic polymers such as polystyrene, polyethylene, polypropylene, polyfluoroethylene, polyethyleneoxy, and polyacrylamide (e.g., polyacrylamide gel), as well as co-polymers and grafts thereof. A substrate may comprise latex or dextran. A substrate may also be inorganic, such as glass, silica, gold, controlled-pore-glass (CPG), or reverse-phase silica. The configuration of a support may be, for example, in the form of beads, spheres, particles, granules, a gel, a porous matrix, or a substrate. In some cases, a substrate may be a single solid or semi-solid article (e.g., a single particle), while in other cases a substrate may comprise a plurality of solid or semi-solid articles (e.g., a collection of particles). Substrates may be planar, substantially planar, or non-planar. Substrates may be porous or non-porous and may have swelling or non-swelling characteristics. A substrate may be shaped to comprise one or more wells, depressions, or other containers, vessels, features, or locations. A plurality of substrates may be configured in an array at various locations. A substrate may be addressable (e.g., for robotic delivery of reagents), or by detection approaches, such as scanning by laser illumination and confocal or deflective light gathering. For example, a substrate may be in optical and/or physical communication with a detector. Alternatively, a substrate may be physically separated from a detector by a distance. An amplification substrate can be placed within or on another substrate, for example, where beads used as amplification substrates are disposed (e.g., immobilized) on a planar surface, or where beads used as amplification substrates are disposed (e.g., immobilized) inside of wells.

The term “coupled to,” as used herein, generally refers to an association between two or more objects that may be temporary or substantially permanent. A first object may be reversibly or irreversibly coupled to a second object. For example, a nucleic acid molecule may be reversibly coupled to a particle or support. A reversible coupling may comprise, for example, a releasable coupling (e.g., in which a first object may be released from a second object to which it is coupled). A first object releasably coupled to a second object may be separated from the second object, e.g., upon application of a stimulus, which stimulus may comprise a photostimulus (e.g., ultraviolet light), a thermal stimulus, a chemical stimulus (e.g., reducing agent), or any other useful stimulus. Coupling may encompass immobilization to a support (e.g., as described herein). Similarly, coupling may encompass attachment, such as attachment of a first object to a second object. A coupling may comprise any interaction that affects an association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-π interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. For example, a particle may be coupled to a planar support via an electrostatic interaction. In another example, a particle may be coupled to a planar support via a magnetic interaction. In another example, a particle may be coupled to a planar support via a covalent interaction. Similarly, a nucleic acid molecule may be coupled to a particle via a covalent interaction. Alternatively or additionally, a nucleic acid molecule may be coupled to a particle via a non-covalent interaction. A coupling between a first object and a second object may comprise a labile moiety, such as a moiety comprising an ester, vicinal diol, phosphodiester, peptidic, glycosidic, sulfone, Diels-Alder, or similar linkage. The strength of a coupling between a first object and a second object may be indicated by a dissociation constant, Kd, that indicates the inclination of a coupled object comprising a first object and a second object to dissociate into the uncoupled first and second objects and may be expressed as a ratio of dissociated (e.g., uncoupled) objects to coupled objects. A smaller dissociation constant is generally indicative of a stronger coupling between coupled objects.

Coupled objects and their corresponding uncoupled components may exist in dynamic equilibrium with one another. For example, a solution comprising a plurality of coupled objects each comprising a first object and a second object may also include a plurality of first objects and a plurality of second objects. At a given point in time, a given first object and a given second object may be coupled to one another or the objects may be uncoupled; the relative concentrations of coupled and uncoupled components throughout the solution will depend upon the strength of the coupling between the first and second objects (reflected in the dissociation constant). For example, a binding moiety may be coupled to a nucleic acid molecule to provide a binding complex. In a solution comprising a plurality of binding complexes each comprising a binding moiety coupled to a nucleic acid molecule, the plurality of binding complexes may exist in equilibrium with their constituent nucleic acid molecules and binding moieties. The association between a given nucleic acid molecule and a given binding moiety may be such that, at a given point in time, at least 50%, such as at least 60%, 65%, 70%, 75%, 80%, 85%, 90%, 95%, 98%, or more, of the nucleic acid molecules may be components of a binding complex of the plurality of binding complexes.

The terms “dispense,” “disperse,” and “load onto a substrate” may be used interchangeably herein. In some cases, dispensing may comprise dispersing and/or dispersing may comprise dispensing. Dispensing generally refers to distributing, depositing, providing, or supplying a reagent, solution, or other object, etc. In some instances, a reagent may comprise a sample. Dispensing may comprise dispersing, which may generally refer to spreading.

The term “label,” as used herein, generally refers to a moiety that is capable of coupling with a species, such as, for example a nucleotide analog. A label may include an affinity moiety. In some cases, a label may be a detectable label that emits a signal (or reduces an already emitted signal) that can be detected. In some cases, such a signal may be indicative of incorporation of one or more nucleotides or nucleotide analogs. In some cases, a label may be coupled to a nucleotide or nucleotide analog, which nucleotide or nucleotide analog may be used in a primer extension reaction. In some cases, the label may be coupled to a nucleotide analog after a primer extension reaction. The label, in some cases, may be reactive specifically with a nucleotide or nucleotide analog. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP), tris(hydroxypropyl)phosphine (THP) or enzymatically cleavable (e.g., via an esterase, lipase, peptidase or protease). As disclosed herein, the terms cleavable and excisable are used interchangeably. In some cases, the label may be luminescent; that is, fluorescent or phosphorescent. Labels may be quencher molecules. The term “quencher,” as used herein refers to a molecule that can reduce an emitted signal. For example, a template nucleic acid molecule may be designed to emit a detectable signal. Incorporation of a nucleotide or nucleotide analog comprising a quencher can reduce or eliminate the signal, which reduction or elimination is then detected. In some cases, as described elsewhere herein, labelling with a quencher can occur after nucleotide or nucleotide analog incorporation. Dyes and labels may be incorporated into nucleic acid sequences. Dyes and labels may also be incorporated into linkers, such as linkers for linking one or more beads to one another. Non-limiting examples of dyes include SYBR green, SYBR blue, DAPI, propidium iodine, Hoechst, SYBR gold, ethidium bromide, acridine, proflavine, acridine orange, acriflavine, fluorocoumarin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5,, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores, Black Hole Quencher Dyes (Biosearch Technologies) such as BH1-0, BHQ-1, BHQ-3, BHQ-10); QSY Dye fluorescent quenchers (from Molecular Probes/Invitrogen) such QSY7, QSY9, QSY21, QSY35, and other quenchers such as Dabcyl and Dabsyl; Cy5Q and Cy7Q and Dark Cyanine dyes (GE Healthcare); Dy-Quenchers (Dyomics), such as DYQ-660 and DYQ-661; and ATTO fluorescent quenchers (ATTO-TEC GmbH), such as ATTO 540Q, 580Q, 612Q. In some cases, the label may be a type that does not self-quench or exhibit proximity quenching. Non-limiting examples of a label type that does not self-quench or exhibit proximity quenching include Bimane derivatives such as Monobromobimane. The term “proximity quenching,” as used herein, generally refers to a phenomenon where one or more dyes near each other may exhibit lower fluorescence as compared to the fluorescence they exhibit individually. In some cases, the dye may be subject to proximity quenching wherein the donor dye and acceptor dye are within 1 nm to 50 nm of each other.

The terms “cycle” and “flow cycle” are used interchangeably and as used herein generally refer to a process in which a nucleotide flow, a wash flow, and a cleavage flow corresponding to each canonical nucleotide (e.g., dATP, dCTP, dGTP, and dTTP or dUTP, or modified versions thereof) are used (e.g., provided to a sequencing template, as described herein). Multiple cycles may be used to sequence and/or amplify a nucleic acid molecule. The order of nucleotide flows can be varied.

Regarding flows, a nucleotide flow can consist of a mixture of labeled and unlabeled nucleotides or nucleotide analogs (e.g., nucleotides or nucleotide analogs of a single canonical type). For example, a solution comprising a plurality of optically (e.g., fluorescently) labeled nucleotides and a plurality of unlabeled nucleotides may be contacted with, e.g., a sequencing template (as described herein). Alternatively, a flow may include only unlabeled nucleotides or nucleotide analogs. A flow may include a mixture of nucleotide or nucleotide analogs of different types (e.g., A and G). A wash flow (e.g., a solution comprising a buffer) may be used to remove any nucleotides that are not incorporated into a nucleic acid complex (e.g., a sequencing template, as described herein). A cleavage flow (e.g., a solution comprising a cleavage reagent) may be used to remove dye moieties (e.g., fluorescent dye moieties) from optically labeled nucleotides or nucleotide analogs.

The term “analyte” may refer to molecules, cells, biological particles, or organisms. In some instances, a molecule may be a nucleic acid molecule, antibody, antigen, peptide, protein, or other biological molecule obtained from or derived from a biological sample. For example, an analyte may be a nucleic acid molecule. An analyte may originate from, and/or be derived from, a biological sample, such as from a cell or organism (e.g., as described herein). An analyte may be synthetic. An analyte may be a biological analyte. For instance, the biological analyte may be a macromolecule, e.g., a nucleic acid, a carbohydrate, a protein, a lipid, etc. The biological analyte may comprise multiple macromolecular groups, e.g., glycoproteins, proteoglycans, ribozymes, liposomes, etc. The biological analyte may be an antibody, antibody fragment, or engineered variant thereof, an antigen, a cell, a peptide, a polypeptide, etc. In some cases, the biological analyte comprises a nucleic acid molecule. The nucleic acid molecule may comprise at least about 10, 100, 1000, 10,000, 100,000, 1,000,000, 10,000,000, 100,000,000, 1,000,000,000 or more nucleotides. Alternatively or in addition, the nucleic acid molecule may comprise at most about 1,000,000,000, 100,000,000, 10,000,000, 1,000,000, 100,000, 10,000, 1000, 100, 10 or fewer nucleotides. The nucleic acid molecule may have a number of nucleotides that is within a range defined by any two of the preceding values. In some cases, the nucleic acid molecule may also comprise a common sequence, to which an N-mer may bind. An N-mer may comprise 1, 2, 3, 4, 5, or 6 nucleotides and may bind the common sequence. In some cases, the nucleic acid molecules may be amplified to produce a colony of nucleic acid molecules attached to the substrate or attached to beads that may associate with or be immobilized to the substrate. In some instances, the nucleic acid molecules may be attached to beads and subjected to a nucleic acid reaction, e.g., amplification, to produce a clonal population of nucleic acid molecules attached to the beads.

The term “processing an analyte,” as used herein, generally refers to one or more stages of interaction with one more sample substances. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The term “detector,” as used herein, generally refers to a device that is capable of detecting a signal, such as a signal indicative of the presence or absence of an incorporated nucleotide or nucleotide analog. A detector may include optical and/or electronic components that may detect signals. Non-limiting examples of detection methods involving a detector include optical detection, spectroscopic detection, electrostatic detection, and electrochemical detection. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel based techniques, such as, for example, gel electrophoresis. Electrochemical detection methods include, but are not limited to, electrochemical detection of amplified product after high-performance liquid chromatography separation of the amplified products.

The term “open substrate”, as used herein, generally refers to a substantially planar substrate in which a single active surface is physically accessible at any point from a direction normal to the substrate. Substantially planar may refer to planarity at a micrometer level or nanometer level. Alternatively, substantially planar may refer to planarity at less than a nanometer level or greater than a micrometer level (e.g., millimeter level).

### Reducing Particle Aggregation

The present disclosure provides methods, compositions, kits, and systems for processing particles. The methods, compositions, kits, and systems provided herein may be useful in reducing aggregation of particles comprising nucleic acid molecules coupled thereto, which may improve downstream analysis including, for example, nucleic acid sequencing. As disclosed herein, reduction of aggregation of particles after applying methods, compositions, kits, and systems disclosed herein can be illustrated by a number of parameters, including, but not limited to a percentage reduction of particles that exist in an aggregate form, an increased number of particles that exist in non-aggregate form, a lower number of particles that exist in an aggregate form, etc.

As disclosed herein, an aggregate exists when multiple particles stick together. An aggregate may be defined by its size and/or by a number of particles forming the aggregate. For example, an aggregate may include 2 or more particles, such as 2, 3, 4, 5, 6, 7, 8, 9, 10, 11, 12, 13, 14, 15, 16, 17, 18, 19, 20, or more particles. Alternatively or additionally, an aggregate may have a dimension exceeding a threshold value, such as a dimension of at least about 1 µm, 1.5 µm, 2 µm, 2.5 µm, 3 µm, 3.5 µm, 4 µm, 4.5 µm, 5 µm, 6 µm, 7 µm, 8 µm, 9 µm, 10 µm, 12 µm, 14 µm, 16 µm, or greater. Such a dimension may be a cross-sectional dimension such as a diameter. Alternatively or additionally, an aggregate may have a cross-sectional area of at least about 0.5 µm², 1 µm², 2 µm², 3 µm², 4 µm², 5 µm², 6 µm², 7 µm², 8 µm², 9 µm², 10 µm², 11 µm², 12 µm², 13 µm², 14 µm², 15 µm², 16 µm², 17 µm², 18 µm², 19 µm², 20 µm², or greater. In some cases, such a cross-sectional area may be approximately circular. In some cases, image analysis may be used to identify the presence of an aggregate. For example, an aggregate may be identified when, e.g., at least 2, 3, 4, 5, 6, 7, 8, 9, 10, 11, 12, 13, 14, 15, 16, 17, 18, 19, 20, or more particles are detected in image analysis (e.g., within a given distance of one another). In some cases, an aggregate may be identified when image analysis detects an object that occupies an area exceeding a threshold value (e.g., including, but not limited to, at least about 0.5 µm², 1 µm², 2 µm², 3 µm², 4 µm², 5 µm², 6 µm², 7 µm², 8 µm², 9 µm², 10 µm², 11 µm², 12 µm², 13 µm², 14 µm², 15 µm², 16 µm², 17 µm², 18 µm², 19 µm², 20 µm², or greater). In some cases, an aggregate may be identified when image analysis detects an object having a dimension that exceeds a threshold value (e.g., including, but not limited to, at least about 1 µm, 1.5 µm, 2 µm, 2.5 µm, 3 µm, 3.5 µm, 4 µm, 4.5 µm, 5 µm, 6 µm, 7 µm, 8 µm, 9 µm, 10 µm, 12 µm, 14 µm, 16 µm, or greater).

For example, a method of the present disclosure may provide a solution or substrate comprising at least 50% fewer aggregates, such as at least 50%, 55%, 60%, 65%, 70%, 75%, 80%, 85%, 90%, 95%, or more fewer particles in an aggregate form when compared to a solution or substrate prepared according to another method (e.g., in the absence of a single-stranded binding moiety). Preferably, a method of the present disclosure provides a solution or substrate comprising at least 95%, 98%, 99%, 99.1%, 99.2%, 99.3%, 99.4%, 99.5%, 99.6%, 99.7%, 99.8%, 99.9%, 99.95%, 99.99%, or fewer particles in an aggregate form when compared to a solution or substrate prepared according to another method (e.g., in the absence of a single-stranded binding moiety). In some cases, a solution or substrate processed according to the method disclosed herein may contain no aggregates. A method, composition, kit, or system of the present disclosure may provide a solution or substrate in which no more than 10%, such as no more than 10%, 9%, 8%, 7%, 6%, 5%, 4%, 3%, 2%, 1%, 0.5%, 0.4%, 0.3%, 0.2%, or fewer, of particles in the solution or coupled to the substrate are in an aggregate form. Preferably, the method, composition, kit, or system of the present disclosure may provide a solution or substrate in which no more than 0.1%, such as no more than 0.09%, 0.08%, 0.07%, 0.06%, 0.05%, 0.04%, 0.03%, 0.02%, 0.01%, 0.005% or fewer, of particles in the solution or coupled to the substrate are in an aggregate form. In some cases, no detectable level of particles exists in aggregate form. Similarly, a method, composition, kit, or system of the present disclosure may provide a solution or substrate in or on which particle aggregates occupying an area of greater than about 20 µm², such as at least about 20 µm², 18 µm², 16 µm², 15 µm², 14 µm², 13 µm², 12 µm², 10 µm², 9 µm², 8 µm², 7 µm², 6 µm², 5 µm², 4 µm², 3 µm², 2 µm², or smaller, are absent or below a predetermined threshold. For example, aggregates occupying an area larger than 8 µm² may constitute about 0.1% or less of the entire particle population.

In an aspect, the present disclosure provides a method for processing a particle of a plurality of particles, such as a bead of a plurality of beads. The method may comprise providing a plurality of particles comprising a first particle (e.g., a first bead) and a second particle (e.g., a second bead). The first particle may comprise a first nucleic acid molecule (e.g., a deoxyribonucleic acid (DNA) or ribonucleic acid (RNA) molecule) coupled (e.g., immobilized) thereto. The first nucleic acid molecule may comprise a first single-stranded portion. In some cases, the first nucleic acid molecule may comprise only a single strand. The method may comprise the first nucleic acid molecule may be contacted with a first binding moiety (e.g., a first single-stranded binding moiety) under conditions sufficient to couple the first binding moiety to the first single-stranded portion of the first nucleic acid molecule to yield a first treated particle comprising a first blocked nucleic acid molecule coupled (e.g., immobilized) thereto, where the first blocked nucleic acid molecule comprises the first nucleic acid molecule (e.g., the first single-stranded region of the first nucleic acid molecule) coupled to the first binding moiety (e.g., the first single-stranded binding moiety). A single-stranded binding moiety may also be referred to herein as a single strand binding moiety. The first treated particle may then be provided in a solution comprising the second particle. The second particle may be a second treated particle that comprises a second blocked nucleic acid molecule coupled (e.g., immobilized) thereto, where the second blocked nucleic acid molecule comprises a second single-stranded portion of a second nucleic acid molecule coupled to a second single-stranded binding moiety.

In another aspect, the present disclosure provides a method for processing a plurality of particles, such as a plurality of beads. The method may comprise providing a plurality of particles comprising a first particle (e.g., a first bead) and a second particle (e.g., a second bead). The first particle may comprise a first nucleic acid molecule (e.g., a DNA or RNA molecule) coupled (e.g., immobilized) thereto. The first nucleic acid molecule may comprise a first single-stranded portion. The second particle may comprise a second nucleic acid molecule (e.g., a DNA or RNA molecule) coupled (e.g., immobilized) thereto. The second nucleic acid molecule may comprise a second single-stranded portion. In some cases, the first nucleic acid molecule and/or the second nucleic acid molecule may comprise only a single strand. The first nucleic acid molecule may be contacted with a first binding moiety (e.g., a first single-stranded binding moiety) of a plurality of binding moieties (e.g., single strand binding moieties) under conditions sufficient to couple the first binding moiety to the first single-stranded portion of the first nucleic acid molecule to yield a first treated particle comprising a first blocked nucleic acid molecule coupled (e.g., immobilized) thereto, where the first blocked nucleic acid molecule comprises the first nucleic acid molecule (e.g., the first single-stranded portion of the first nucleic acid molecule) coupled to the first binding moiety. At the same time or at a later time, the second nucleic acid molecule may be contacted with a second binding moiety (e.g., a second single-stranded binding moiety) of a plurality of binding moieties (e.g., single strand binding moieties) under conditions sufficient to couple the second binding moiety to the second single-stranded portion of the second nucleic acid molecule to yield a second treated particle comprising a second blocked nucleic acid molecule coupled (e.g., immobilized) thereto, where the second blocked nucleic acid molecule comprises the second nucleic acid molecule (e.g., the second single-stranded portion of the second nucleic acid molecule) coupled to the second binding moiety. The first treated particle and the second treated particle may be prepared in a solution, such that they are included in a same solution subsequent to their formation. Alternatively, the first treated particle and the second treated particle may be prepared in separate solutions or compartments, such as in separate droplets, tubes, vials, or wells. Subsequent to contacting the first nucleic acid molecule and the second nucleic acid molecule with the first binding moiety and the second binding moiety, respectively, the first treated particle and the second treated particle may be included in a same solution. For example, the first treated particle prepared in a first compartment may be combined with the second treated particle prepared in a second compartment. The first treated particle and the second treated particle may be prepared while the first particle and the second particle are coupled to a substrate (e.g., as described herein). Alternatively, the first treated particle and the second treated particle may be immobilized to a substrate subsequent to contacting the first nucleic acid molecule and the second nucleic acid molecule with the first binding moiety and the second binding moiety, respectively. For example, a solution comprising the first treated particle and the second treated particle may be brought into contact with a substrate under conditions sufficient to couple (e.g., immobilize) the first treated particle and/or the second treated particle to the substrate.

In another aspect, the present disclosure provides a method for processing a plurality of particles, such as a plurality of beads. The method may comprise providing a plurality of particles, where each particle of at least a subset of the plurality of particles comprises a nucleic acid molecule (e.g., a DNA or RNA molecule) of a plurality of nucleic acid molecules immobilized thereto. Each nucleic acid molecule of at least a subset of the plurality of nucleic acid molecules may comprise a single-stranded portion. In some cases, one or more nucleic acid molecules of the plurality of nucleic acid molecules may comprise only a single strand. The plurality of particles may be contacted with a plurality of binding moieties (e.g., single strand binding moieties) under conditions sufficient for single-stranded binding moieties of the plurality of single-stranded binding moieties to couple to single-stranded portions of nucleic acid molecules of the at least the subset of the plurality of nucleic acid molecules. Subsequent to this contacting process, the plurality of particles may be included in a solution. The solution may be the same solution in which the plurality of particles is contacted with the plurality of binding moieties or a different solution. In some cases, particles of the plurality of particles may be contacted with the plurality of binding moieties separately, such as in different solutions and/or in separate compartments, such as in separate droplets, tubes, vials, or wells, and at the same or different times. The plurality of particles may be immobilized to a substrate (e.g., as described herein). For example, particles may be immobilized to a substrate prior to being contacted with the plurality of binding moieties, or particles may be immobilized to a substrate subsequent to being contacted with the plurality of binding moieties. In some cases, subsequent to the contacting process, no more than 10% of particles of the plurality of particles may be included in a particle aggregate comprising two or more particles of the plurality of particles. For example, no more than 9%, 8%, 7%, 6%, 5%, 4%, 3%, 2%, 1%, 0.5%, 0.25%, 0.1%, 0.05%, 0.02%, 0.01%, 0.008%, 0.005%, 0.002%, 0.001%, or fewer particles may be included in a particle aggregate. In an example, no more than 1% of particles of the plurality of particles may be included in a particle aggregate comprising two or more particles of the plurality of particles. In another example, no more than 0.5% of particles of the plurality of particles may be included in a particle aggregate comprising two or more particles of the plurality of particles. In a further example, no more than 0.1% of particles of the plurality of particles may be included in a particle aggregate comprising two or more particles of the plurality of particles. Alternatively or additionally, in some cases, subsequent to the contacting process, particle aggregates, comprising two or more particles of the plurality of particles (e.g., such as at least 2, 3, 4, 5, 6, 7, 8, 9, 10, or more particles), having a dimension of at least about 5 µm may be absent from the solution in which the plurality of particles are included. For example, aggregates having a dimension of at least about 1 µm, 1.5 µm, 2 µm, 2.5 µm, 3 µm, 3.5 µm, 4 µm, 4.5 µm, 5 µm, 6 µm, 7 µm, 8 µm, 9 µm, 10 µm, 12 µm, 14 µm, 16 µm, or greater may be absent from the solution. In an example, aggregates having a dimension of at least about 3 µm may be absent from the solution. Such a dimension may be, for example, a cross-sectional dimension such as a diameter.

A plurality of particles may be a plurality of beads, such as a plurality of polymer or gel beads. A plurality of particles may comprise at least 2 particles (e.g., at least first and second particles), such as at least 10, 20, 50, 100, 1,000, 2,000, 5,000, 10,000, 20,000, 30,000, 50,000, 100,000, 150,000, 200,000, 500,000, 1,000,000, 10,000,000, 20,000,000, 50,000,000, 100,000,000, or more particles. For example, the plurality of particles may comprise at least 10 million, 20 million, 50 million, 100 million, 200 million, 500 million, 600 million, 700 million, 800 million, 900 million, 1 billion, 1.2 billion, 1.5 billion, 2 billion, 2.5 billion, 3 billion, 4 billion, 5 billion, 6 billion, 7 billion, 8 billion, 9 billion, 10 billion, 11 billion, 12 billion, 13 billion, 14 billion, 15 billion, 16 billion, 17 billion, 18 billion, 19 billion, 20 billion, or more particles. A suitable number of particles may be selected based on particle size, particle shape, substrate size, and substrate shape. Particles may have any useful structure, size, and characteristics. A particle may be approximately spherical or may have a dumbbell shape, an oblong or elliptical shape, a rectangular prism shape, or any other shape. In some cases, a particle may have an irregular shape. Particles of a plurality of particles may have the same or approximately the same shape. Alternatively, a plurality of particles may comprise particles having a variety of shapes, such as a variety of irregular shapes. Particles of a plurality of particles may have any useful dimension. For example, a particle may have a dimension (e.g., a diameter or cross-section) of at least about 10 nm, 25 nm, 50 nm, 100 nm, 200 nm, 300 nm, 400 nm, 500 nm, 600 nm, 700 nm, 800 nm, 900 nm, 1 µm, 1.2 µm, 1.5 µm, 2 µm, 2.5 µm, 3 µm, 4 µm, 5 µm, 10 µm, 20 µm, 30 µm, 40 µm, 50 µm, 60 µm, 70 µm, 80 µm, 90 µm, 100 µm, 250 µm, 500 µm, 1 millimeter (mm), or greater. In some cases, a particle may have a dimension (e.g., a diameter or cross-section) of no more than about 10 nm, 25 nm, 50 nm, 100 nm, 200 nm, 300 nm, 400 nm, 500 nm, 600 nm, 700 nm, 800 nm, 900 nm, 1 µm, 1.2 µm, 1.5 µm, 2 µm, 2.5 µm, 3 µm, 4 µm, 5 µm, 10 µm, 20 µm, 30 µm, 40 µm, 50 µm, 60 µm, 70 µm, 80 µm, 90 µm, 100 µm, 250 µm, 500 µm, 1 mm, or less. In some cases, a particle may have a dimension (e.g., diameter or cross-section) in the range of about 10-100 nm, 10-500 nm, 100-1000 nm, 500 nm - 5 µm, 1-50 µm, 10-100 µm, 50-100 µm, 1-500 µm, 10-500 µm, 100-500 µm, 1-1000 µm, 10-1000 µm, 100-1000 µm, or any range therein. As disclosed herein, the size of a particle may be comparable to or larger than the smallest unit that can be resolved optically in image analysis (e.g., the size of a pixel, such as a pixel of an imaging device). Preferably, a particle may have a dimension between 100 nm to 5 µm. In an example, a particle may have a dimension between about 0.5-0.8 µm. In another example, a particle may have a dimension between about 0.8-1.2 µm. Particles of a plurality of particles may have the same or approximately the same size. In some cases, a plurality of particles may have an average dimension according to the sizes provided thereof. For example, a plurality of particles may have a relatively monodisperse size distribution. In another example, a plurality of particles may have a polydisperse size distribution. For example, a plurality of particles may comprise particles having a first average dimension and particles having a second average dimension which second average dimension is greater than the first average dimension. In some cases, a plurality of particles may have a size distribution that has a coefficient of variation in a dimension of less than about 50% such as less than about 40%, 30%, 20%, 10%, 5%, 1%, or less.

Particles of a plurality of particles may be, for example, flexible, compressible, semi-rigid, or rigid. A particle may be solid, semi-solid, semi-fluidic, fluidic, or a combination thereof. A particle may be porous or non-porous. A particle may be at least partially degradable or dissolvable, such as upon application of a stimulus (e.g., a thermal stimulus, chemical stimulus (e.g., reducing agent), a photo stimulus (e.g., UV light), a pH change, etc.). A particle may comprise synthetic and/or natural materials. A particle may comprise a polymeric material, such as one or more different polymeric materials. A particle may comprise a gel such as a hydrogel. For example, a particle may comprise a cross-linked polymer. A particle may comprise a plurality of disulfide linkages linking, e.g., monomers, oligomers, or polymers. In an example, a particle may comprise polyacrylamide that has been polymerized in the presence of cystamine or a similar molecule to provide a particle comprising a polyacrylamide particle comprising disulfide linkages. Such a particle may be at least partially degradable or dissolvable (e.g., by reduction of disulfide linkages). A particle may comprise an acrydite moiety. A particle may comprise a detectable material, such as a fluorescent material. A particle may comprise a magnetic or paramagnetic material. A particle may comprise a metal such as iron oxide, silica, gold, or silver. A particle may comprise a material embedded within the particle, such as within pores of the particle and/or coupled to a polymeric network of the particle. For example, an analyte and/or reagent, including a detectable and/or magnetic material, may be embedded within the particle. Alternatively or additionally, a particle may comprise a material coupled to the exterior of the particle. For example, a particle may be coupled to a detectable moiety, such as a molecule comprising a detectable label (e.g., as described herein). Particles of a plurality of particles may comprise the same materials. For example, each particle of a plurality of particles may be prepared from the same materials and according to a same method. Alternatively, a plurality of particles may comprise particles comprising one or more different materials. For example, a plurality of particles may comprise a first subset of particles comprising a first material and a second subset of particles comprising a second material that is different from the first material. Similarly, each particle of a plurality of particle may comprise a same material or moiety, such as a magnetic material or a detectable moiety. Alternatively, a plurality of particles may comprise particles that comprise a given material or moiety as well as particles that do not comprise the given material or moiety. In an example, a plurality of particles comprises a first subset of particles comprising a magnetic or paramagnetic material and second subset of particles that do not comprise a magnetic or paramagnetic material. In another example, a plurality of particles comprises a first subset of particles comprising a detectable moiety and a second subset of particles that do not comprise a detectable moiety.

A particle may be coupled to a nucleic acid molecule (e.g., as described herein). A nucleic acid molecule may be coupled to a particle via a covalent bond. For example, a nucleic acid molecule coupled to a particle may be coupled to the particle via a disulfide moiety. A disulfide moiety may be linked to, e.g., an acrydite moiety of the particle. For example, a particle may comprise an acrydite moiety coupled to a thiol or reactive hydroxyl moiety that may serve as an attachment mechanism for a nucleic acid molecule. A nucleic acid molecule coupled to a particle may be completely external to the particle. Alternatively, at least a portion of a nucleic acid molecule coupled to a particle may be internal to the particle. A nucleic acid molecule coupled to a particle may be immobilized to the particle.

A nucleic acid molecule (e.g., a nucleic acid molecule coupled to a particle) may be derived from a sample, such as a biological sample. A nucleic acid molecule may be derived from a cell, such as a cell of a sample. A nucleic acid molecule may comprise a sequence of a sample nucleic acid molecule. A nucleic acid molecule may be a synthetic nucleic acid molecule, and/or may comprise a synthetic sequence. A nucleic acid molecule may have any useful features and be of any useful type. For example, a nucleic acid molecule may comprise deoxyribonucleic acid (DNA) and/or ribonucleic acid (RNA). For instance, a nucleic acid molecule may comprise human genomic DNA. A nucleic acid molecule may be at least partially double-stranded. For example, a nucleic acid molecule may comprise one or more double-stranded regions separated by one or more single-stranded regions. A nucleic acid molecule may comprise one or more single-stranded regions. For example, a nucleic acid molecule may comprise a single-stranded region distal to an end of the nucleic acid molecule coupled to a particle. In some cases, a nucleic acid molecule may include only a single strand. A single-stranded region of a nucleic acid molecule may have any useful size and characteristics. For example, a single-stranded region of a nucleic acid molecule may comprise at least 2 nucleotides, such as at least 5, 10, 15, 20, 30, 40, 50, 60, 70, 80, 90, 100, or more nucleotides. In some cases, a nucleic acid molecule may not initially comprise a single-stranded region, or may not comprise a single-stranded region of interest. For example, a nucleic acid molecule may initially be double-stranded. A method of preparing a treated particle (e.g., a particle comprising a blocked nucleic acid molecule) may comprise providing a single-stranded region of a nucleic acid molecule coupled to a particle, such as via denaturing a region of a double-stranded region (e.g., as described herein), excision and/or dissociation of one or more nucleotides of a strand of a double-stranded region (e.g., as described herein), and/or enzymatic action upon a region of a double-stranded region (e.g., using an endonuclease, nickase, exonuclease, or other enzyme).

A nucleic acid molecule may have any useful length and composition. For example, a nucleic acid molecule may comprise at least 10 nucleotides in a given strand, such as at least 10, 20, 30, 40, 50, 60, 70, 80, 90, 100, 150, 200, 250, 300, 350, 400, 450, 500, 600, 700 800, 900, 1,000 or more nucleotides in a given strand. A nucleic acid molecule may include canonical DNA and RNA nucleotides (e.g., adenine, cysteine, thymine, uracil, and guanine) and/or nucleotide analogs (e.g., as described herein), including nucleotides and nucleotide analogs coupled to labeling moieties (e.g., as described herein). In some cases, a nucleic acid molecule may be enriched in one or more nucleotides or nucleotide analogs. For example, a nucleic acid molecule may comprise a region that is enriched in adenine, thymine, inosine, and/or similar nucleotides or nucleotide analogs. Such a region may have a melting temperature that differs from melting temperatures of other regions of a nucleic acid molecule, which may facilitate selective denaturation of a strand of a region of a double-stranded nucleic acid molecule upon application of thermal energy. A nucleic acid may include one or more functional sequences. For example, a nucleic acid molecule may comprise a sequencing primer sequence, a flow cell attachment sequence, a priming sequence (e.g., a random N-mer or targeted priming sequence, such as a poly(thymine) sequence), a capture sequence, a unique molecular identifier sequence, a barcode sequence, or any other useful sequence, or a complement of any such sequence. Such sequences may be included in a single-stranded region of a nucleic acid molecule. A nucleic acid molecule may comprise an adapter, such as a single- or double-stranded adapter. A nucleic acid molecule coupled to a particle may be releasably coupled to the particle. For example, a nucleic acid molecule may be releasable from a particle to which it is coupled upon application of a stimulus, such as a thermal stimulus, chemical stimulus (e.g., reducing agent), photo stimulus (e.g., UV light), pH change, etc. For example, a nucleic acid molecule coupled to a particle via a disulfide linkage may be releasable from the particle upon cleavage of the disulfide bond. In another example, a nucleic acid molecule may be releasable from a particle to which it is coupled upon partial or complete dissolution or degradation of the particle. Alternatively, a nucleic acid molecule may not be releasable from a particle to which it is coupled.

A nucleic acid molecule may comprise a cleavable or excisable moiety in one or more strands. For example, a nucleic acid molecule may comprise a cleavable or excisable moiety near an end of the nucleic acid molecule, such as an end of the nucleic acid molecule that is distal to an end coupled to a particle. A cleavable or excisable moiety (e.g., a cleavable base) may be a moiety such as a nucleobase that may be cleaved and/or excised from a nucleic acid molecule. Examples of cleavable or excisable moieties include, but are not limited to, uracil, 8-oxoguanine (also referred to as 8-hydroxyguanine, 8-oxo-7,8-dihydroguanine, 7,8-dihydro-8-oxoguanine, and 8oxoG herein), inosine, and 2,6-diamino-4-hydroxy-5-formamidopyrimidine (FapyG). Cleavage and/or excision of a cleavable or excisable moiety may be carried out by contacting the cleavable or excisable moiety (e.g., cleavable base) with a cleaving agent. Examples of cleaving agents include, but are not limited to, uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), and RNase (e.g., RNaseH, such as RNaseHII). Photocleavable or photoexcisable moieties may be cleaved or excised using appropriate application of energy, such as by contacting the moiety with UV light. One or more cleaving agents may be used in combination to cleave or excise a cleavable or excisable moiety. In an example, the cleavable base may be an RNA base in a DNA backbone, and the cleaving agent may be RNase (e.g., RNaseH or RNaseHII). In such a case, the nucleic acid molecule may not be an RNA molecule. In another example, the cleavable base may be an uracil base and the cleaving agent may be selected from uracil DNA glycosylase (UDG), apyrimidinic/apurinic endonuclease (APE), Endonuclease VIII and uracil-specific excision reagent (USER) enzyme. For example, the cleaving agent may be UDG. For example, the cleaving agent may be APE. In another example, the cleavable base may be an inosine base and the cleaving agent may be Endonuclease V (Endo V). In another example, the cleavable base may be 2,6-diamino-4-hydroxy-5-formamidopyrimidine (FapyG) base and the cleaving agent may be formamidopyrimidine DNA glycosylase (Fpg). In another example, the cleavable base may be 8-oxo-7,8-dihydroguanine (8oxoG) and the cleaving agent may be 8-oxoguanine glycosylase (OGG1). In another example, the cleavable base may be a photo-cleavable base and the cleaving agent may be light, such as laser light. Application of a cleaving agent may generate a “nick” in a strand of a nucleic acid molecule. Alternatively or additionally, another enzyme may be added to generate a nick, or otherwise functionalize a nick. For example, T4 PNK may be added to remove a 3′ phosphate. An enzyme may be used to remove a lesion, such as a 3′ lesion. In an example, a nucleic acid molecule may comprise a cleavable or excisable moiety in a first strand of a double-stranded region at an end of the nucleic acid molecule that is distal to an end coupled to a particle. The cleavable or excisable moiety may be cleaved or excised to provide a gap or nicked region in the first strand. In some cases, the first strand of the double-stranded region may comprise multiple cleavable or excisable moieties such that cleavage or excision of the multiple cleavable moieties may provide multiple gaps or nicked regions or a gap or nicked region spanning more than one nucleotide. Additional nucleotides may dissociate upon introduction of a nearby gap or nicked region. In some cases, an enzyme may be used to expand the size of a gap or nicked region. Such processes may provide a single-stranded region, for example, at the distal end of the nucleic acid molecule, which single-stranded region may be used in additional processing as described herein.

A particle may comprise a plurality of nucleic acid molecules coupled thereto. For example, a particle may comprise two or more nucleic acid molecules coupled thereto, such as at least 2, 10, 20, 50, 100, 200, 500, 1,000, 2,000, 5,000, 10,000, 50,000, 100,000, 200,000, 500,000, 1,000,000, 2,000,000, 5,000,000, 10,000,000, or more nucleic acid molecules. For example, a particle may comprise at least 1,000 nucleic acid molecules coupled thereto. In another example, a particle may comprise at least 100,000 nucleic acid molecules coupled thereto. Nucleic acid molecules coupled to a given particle may have identical nucleic acid sequences (e.g., sequences of different nucleic acid molecules may have sequence identity to one another). Alternatively or additionally, nucleic acid molecules coupled to a particle may have different nucleic acid sequences. Nucleic acid molecules coupled to a particle may comprise both identical and different nucleic acid sequences. For example, nucleic acid molecules coupled to a particle may comprise identical barcode sequences and, in some cases, other sequences but may comprise unique molecular identifier sequences that vary across the population of nucleic acid molecules coupled to the particle. In another example, a plurality of nucleic acid molecules coupled to a particle may comprise a first subset of nucleic acid molecules having a first nucleic acid sequence and a second subset of nucleic acid molecules having a second nucleic acid sequence, where the first and second nucleic acid sequences are not identical to one another. For example, a plurality of nucleic acid molecules coupled to a particle may comprise a first subset of nucleic acid molecules having a first capture or primer sequence and a second subset of nucleic acid molecules having a second capture or primer sequence, where the first and second capture or primer sequences are not identical to one another.

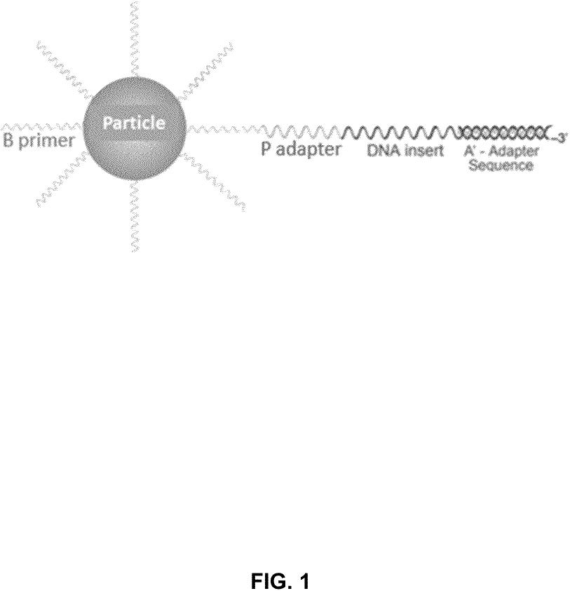

FIG. 1 shows an example of a particle having a nucleic acid molecule coupled thereto. The particle may comprise properties of any of the particles described elsewhere herein. For example, the particle may have any useful size, shape, or other characteristic. The particle may comprise any useful material. The particle may be a bead, such as a gel bead. The particle may comprise a magnetic material and/or a detectable moiety. The particle in the figure has a plurality of nucleic acid molecules coupled thereto, but the number of nucleic acid molecules displayed is not intended to be limiting. A particle may comprise any useful number of nucleic acid molecules coupled thereto. For example, a particle may include a single nucleic acid molecule or a plurality of nucleic acid molecules coupled thereto, such as greater than 100 nucleic acid molecules coupled thereto (e.g., as described herein). For example, a particle may have at least 1,000 nucleic acid molecules, such as at least 100,000 nucleic acid molecules, coupled thereto. Similarly, though two different types of nucleic acid molecules are shown, any useful number of nucleic acid molecules having different nucleic acid sequences and features may be used in any desired proportion. A particle may comprise a first plurality of nucleic acid molecules of a first type and second plurality of nucleic acid molecules of a second type that is different than the first type. For example, a particle may comprise a single extended nucleic acid molecule and a plurality of unextended primer nucleic acid molecules. Nucleic acid molecules coupled to the particle may be single- or double-stranded nucleic acid molecules. For example, at least a subset of nucleic acid molecules coupled to the particle may be single-stranded nucleic acid molecules. In FIG. 1, most nucleic acid molecules coupled to the particle are shown as unextended primer nucleic acid molecules (“B primers”). The right-most nucleic acid molecule shown in the figure includes the primer sequence as well as additional sequences. While an adapter sequence and a sequence of a template or sample nucleic acid molecule (“DNA insert” sequence) are shown, one or more additional sequences, including spacer sequences, barcode sequences, unique identifier sequences, adapter sequences, and/or other sequences, may also be included at any useful location, including between the particle and the primer sequence. Such sequences may be included in single-stranded regions of a nucleic acid molecule coupled to a particle and/or in double-stranded regions of a nucleic acid molecule coupled to a particle. In the extended primer shown in FIG. 1, the nucleic acid molecule comprises a double-stranded region distal to the particle that comprises an adapter sequence A coupled to a complementary sequence A′. The hybridized A′ sequence may function as a blocking oligomer to prevent the A sequence from hybridizing to other sequences, including to other sequences coupled to other particles included in a solution, thereby preventing formation of a particle aggregate including the treated particle. Similarly, the hybridized A′ sequence may prevent the A sequence from hybridizing to other nucleic acid molecules that may be included in a solution comprising the treated particle, including nucleic acid contaminants. The use of the blocking oligomers may therefore function to reduce aggregation and contamination of samples, which may result in improved sequencing quality in downstream analysis. Other binding moieties such as single-strand binding proteins may also be used in place of blocking oligomers (e.g., as described herein). Treatment of a particle as described herein may comprise contacting a nucleic acid molecule comprising a single-stranded region with a single-stranded binding moiety (e.g., as described herein). The single-stranded binding moiety may be configured to associate with all or a portion of the single-stranded region of the nucleic acid molecule. For example, the single-stranded binding moiety may be configured to associate with the sequences labeled “P1 sequence” and “DNA insert” of the nucleic acid molecule in the figure.

A binding moiety may be a single-stranded binding moiety (e.g., a binding moiety configured to bind a single strand of a nucleic acid molecule). A binding moiety may be configured to bind to a specific sequence of a nucleic acid molecule, such as a sequence of a single-stranded region of a nucleic acid molecule.

A single-stranded binding moiety may comprise a single-stranded binding (SSB) protein. SSB proteins may be useful for stabilizing a single-stranded region of a nucleic acid molecule, such as a DNA molecule. An SSB protein may derive from a bacterium. For example, an SSB protein may derive from Escherichia coli (E. coli). An SSB protein may derive from phage T4 (e.g., a T4 Gene 32 Protein or T4 SSB protein). Additional examples of SSB proteins include human replication protein A (hRPA) SSB protein, human SSB1 protein, and Extreme Thermostable SSB protein (New England BioLabs). In some cases, a portion of an SSB protein may be used, such as a truncated SSB protein. In other examples, a binding moiety may comprise a nucleic acid (e.g., DNA) binding protein such as a transcription factor, a polymerase, a nuclease, or a histone. A single-stranded binding protein may have any useful features. An SSB protein may comprise a tetramer, such as a tetramer having a footprint of approximately 30 bases. In such an instance, approximately 300 ng/million particles may be a saturating amount of SSB protein.

In some cases, a single-stranded binding moiety may comprise a nucleic acid molecule. Such a nucleic acid molecule may comprise a random N-mer (e.g., a random sequence including N nucleotides). A random N-mer may comprise, for example, between 6 and 10 nucleotides, such as 6 nucleotides (e.g., a random hexamer). A population of random N-mers may comprise a plurality of nucleic acid molecules, each of which has a different nucleic acid sequence. Alternatively, a population of random N-mers may comprise a plurality of nucleic acid molecules, one or more of which may comprise the same nucleic acid sequence. The population of random N-mers may be such that it is statistically unlikely that random N-mers having the same nucleic acid sequence will come into contact with one another. For example, a population of random N-mers may comprise at least 100, 500, 1,000, 2,000, 5,000, 10,000, 50,000, 100,000, 500,000, 1,000,000, 5,000,000, 10,000,000, or more nucleic acid molecules. In some cases, a population of random N-mers may comprise at least 100, 500, 1,000, 2,000, 5,000, 10,000, 50,000, 100,000, 500,000, 1,000,000, 5,000,000, 10,000,000, or more different nucleic acid sequences. In some cases, at least about 50%, such as at least 55%, 60%, 65%, 70%, 75%, 80%, 85%, 90%, 95%, or more, of a population of random N-mers may comprise different nucleic acid sequences.

A single-stranded binding moiety may comprise a nucleic acid molecule comprising a sequence having sequence complementarity to a sequence of a nucleic acid molecule coupled to a particle. Such a nucleic acid molecule may alternately be referred to as a “blocking oligonucleotide” or “blocking nucleic acid molecule.” For example, a particle comprising a first nucleic acid molecule coupled thereto, where the first nucleic acid molecule comprises a first sequence, may be contacted with a single-stranded binding moiety that comprises a second nucleic acid molecule comprising a second sequence (e.g., a blocking oligonucleotide or blocking nucleic acid molecule), where the second sequence has at least partial sequence complementarity to the first sequence. In some cases, the first sequence may comprise mainly a surface primer sequence. In some cases, the first sequence may comprise a surface primer sequence and a target nucleic acid sequence. In some cases, the first sequence may comprise a surface primer sequence and a target nucleic acid sequence, where the target nucleic acid sequence is flanked by adapter sequences on both sides. In some cases, the second sequence can be the strand that is not covalently attached to a particle; for example, amplified nucleic acid molecules may be kept in double-stranded form to minimize aggregation prior to performance of a sequencing process. A sequence of a nucleic acid molecule that acts as a single-stranded binding moiety may have at least 50% sequence complementarity to a sequence of a nucleic acid molecule coupled to a substrate or particle (e.g., a target sequence), such as at least 55%, 60%, 65%, 70%, 75%, 80%, 85%, 90%, 95%, 96%, 97%, 98%, 99%, or greater. A sequence of a nucleic acid molecule may have at least partial sequence complementarity to a target sequence disposed at or near a terminus of a nucleic acid molecule coupled to a substrate or particle, such as target sequence distal from a substrate or particle. Alternatively or additionally, a sequence of a nucleic acid molecule may have at least partial sequence complementarity to a target sequence that is not disposed at or near a terminus of a nucleic acid molecule coupled to a substrate or particle, such as a target sequence included in a single-stranded region of a nucleic acid molecule that may be flanked by at least one double-stranded region. A nucleic acid molecule that acts as a single-stranded binding moiety may be a single-stranded nucleic acid molecule. Alternatively, a nucleic acid molecule that acts as a single-stranded binding moiety may comprise at least one double-stranded region (e.g., a hairpin-like structure). In an example, a nucleic acid molecule may comprise a first strand including a sequence complementary to a target sequence and a second strand that is coupled to the first strand, and hybridization of the first strand or a portion thereof to the target sequence may be accompanied and/or preceded by separation of the first strand and the second strand, or portions thereof. A sequence of a nucleic acid molecule that acts as a single-stranded binding moiety may be included in a portion of the nucleic acid molecule that has a low melting point, such as a melting temperature that is lower than that of another region of the nucleic acid molecule. For example, the sequence may be included in a portion of the nucleic acid molecule having a melting temperature of less than about 65° C., such as less than about 60° C., 55° C., 50° C., 45° C., 40° C., 35° C., or lower. A sequence of a nucleic acid molecule that acts as a single-stranded binding moiety may be of any useful length and may have any useful composition. For example, a sequence of a nucleic acid molecule that acts as a single-stranded binding moiety may comprise at least 2 nucleotides, such as least 3, 4, 5, 6, 7, 8, 9, 10, 12, 14, 16, 18, 20, or more nucleotides. A sequence of a nucleic acid molecule that acts as a single-stranded binding moiety may have the same or a different length as a sequence to which it may configured to bind, such as a primer or capture sequence. For example, a sequence of a nucleic acid molecule that acts as a single-stranded binding moiety may comprise fewer nucleotides than a primer or capture sequence which it may be configured to bind. A sequence of a nucleic acid molecule that acts as a single-stranded binding moiety may comprise a sequence that is configured to be completely complementary to a target sequence. Alternatively, a sequence of a nucleic acid molecule that acts as a single-stranded binding moiety may comprise a sequence that is configured to be less than 100% complementary to a target sequence, such as less than 99%, 98%, 97%, 96%, 95%, 90%, 85%, 80%, or less. In some cases, the sequence of the nucleic acid molecule that acts as a single-stranded binding moiety may be configured such that, when the sequence is bound to a target sequence, one or more nucleotides of the nucleic acid molecule that acts as a single-stranded binding moiety may not be associated (e.g., via noncovalent interactions including hydrogen bonds) with a nucleotides of the target sequence (e.g., there may one or more base mismatches). A sequence of a nucleic acid molecule that acts as a single-stranded binding moiety may include any useful composition. For example, the sequence may include canonical nucleotides and/or nucleotide analogs. The sequence may be enriched in one or more nucleotides or nucleotide analogs. For example, a nucleic acid molecule may comprise a region that is enriched in adenine, thymine, inosine, and/or similar nucleotides or nucleotide analogs.

A nucleic acid molecule that acts as a single-stranded binding moiety (e.g., a blocking oligonucleotide) may comprise a linear structure. A nucleic acid molecule that acts as a single-stranded binding moiety may also or alternatively comprise a secondary structure. For example, a nucleic acid molecule that acts a s a single-stranded binding moiety may comprise one or more branchpoints, loop structures, hairpins, stem-loops, helices, or any other useful secondary structure. A secondary structure may be included in or branched off of a portion of the nucleic acid molecule that is configured to couple to a nucleic acid molecule coupled to a particle. Alternatively or additionally, a secondary structure such as a loop (e.g., hairpin moiety) may be external to a portion of the nucleic acid molecule that is configured to couple to a nucleic acid molecule coupled to a particle. For example, the nucleic acid molecule that acts as a single-stranded binding moiety may comprise a single-stranded portion configured to couple to a nucleic acid molecule coupled to a particle, which single-stranded portion is coupled to a loop or stem-loop structure. Such a loop structure may facilitate stabilization of the blocking oligonucleotide coupled to the nucleic acid molecule coupled to the particle, for instance, upon contacting the nucleic acid molecule coupled to the particle with additional SSB moieties (e.g., SSB proteins) at a later time.

A nucleic acid molecule that acts as a single-stranded binding moiety may comprise one or more additional sequences in addition to a sequence having at least partial sequence complementarity to a target sequence. For example, a nucleic acid molecule that acts as a single-stranded binding moiety may comprise a tag or index sequence such as a barcode sequence or unique molecular identifier sequence. Alternatively or additionally, a nucleic acid molecule that acts as a single-stranded binding moiety may comprise any other functional sequence described herein, including a spacer sequence, linker sequence, adapter sequence, or any other sequence, any of which may be included in a double-stranded region of the nucleic acid molecule.

A single-stranded binding moiety (e.g., an SSB protein or nucleic acid molecule) may comprise a tag or label. For example, a single-stranded binding moiety may comprise an index, tag, or barcode sequence or a unique molecular identifier sequence. Such a sequence may be incorporated into or appended to, for example, a nucleic acid molecule that acts as a single-stranded binding moiety. Similarly, a nucleic acid molecule that acts as a single-stranded binding moiety may be configured to bind one or more probe molecules, which one or more probe molecules may comprise optically detectable labels. In an example, a particle (e.g., bead) coupled to (e.g., immobilized to) a nucleic acid molecule comprising a target sequence (e.g., as described herein) is contacted with a single-stranded binding moiety that comprises a nucleic acid molecule comprising a sequence having at least partial sequence complementarity to the target sequence under conditions sufficient for the sequence of the single-stranded binding moiety to couple to the target sequence of the nucleic acid molecule coupled to the particle. The single-stranded binding moiety coupled to the particle may then be contacted with a plurality of probes, where a probe of the plurality of probes is configured to hybridize to a sequence of the single-stranded binding moiety. The probe may comprise an optically detectable moiety such as a fluorescent dye moiety (e.g., as described herein). Multiple probes may be configured to hybridize to different sequences of the single-stranded binding moiety, where each probe may comprise a different fluorescent dye moiety and/or be configured to fluoresce at a different intensity. Subsequent excitation and imaging may facilitate identification of particles to which a single-stranded binding moiety has coupled. Alternatively or additionally, a single-stranded binding moiety may comprise an optically detectable moiety. For example, a nucleic acid molecule that acts as a single-stranded binding moiety may comprise an optically detectable moiety coupled thereto. In another example, an SSB protein may comprise an optically detectable moiety coupled thereto. Alternatively or additionally, a particle may comprise an optically detectable moiety. An optically detectable moiety may comprise a fluorescent dye moiety (e.g., as described herein). Excitation of the optically detectable moiety and subsequent imaging may facilitate identification of a particle to which a single-stranded binding moiety has coupled (e.g., subsequent to a washing process configured to remove un-coupled single-stranded binding moieties).

In an example, a first particle (e.g., a bead) may comprise a first nucleic acid molecule (e.g., a deoxyribonucleic acid (DNA) or ribonucleic acid (RNA) molecule) coupled (e.g., immobilized) thereto. The first nucleic acid molecule may comprise a first single-stranded portion. In some cases, the first nucleic acid molecule may comprise only a single strand. The method may comprise contacting the first nucleic acid molecule with a first binding moiety (e.g., a first single-stranded binding moiety, such as a nucleic acid molecule or an SSB protein) under conditions sufficient to couple the first binding moiety or portion thereof to the first single-stranded portion of the first nucleic acid molecule to yield a first treated particle comprising a first blocked nucleic acid molecule coupled (e.g., immobilized) thereto, where the first blocked nucleic acid molecule comprises the first nucleic acid molecule (e.g., the first single-stranded region of the first nucleic acid molecule) coupled to the first binding moiety (e.g., the first single-stranded binding moiety) or portion thereof. The first binding moiety may comprise an optically detectable moiety, such as a fluorescent dye moiety. The method may further comprise detecting the optically detectable moiety of the first binding moiety coupled to the treated particle. Prior to detecting the optically detectable moiety, the treated particle may undergo processing including filtration, isolation, separation, sequestration, and/or enrichment (e.g., as described herein). For example, the treated particle may be separated from other untreated particles included in a solution comprising a plurality of particles including both treated and untreated particles. The treated particle may be immobilized to a support (e.g., as described herein). Detection of an optically detectable moiety coupled to the treated particle may be used to determine that the particle has been treated according to the method described herein, and/or to identify the particle within a solution and/or coupled to a support.

Any useful amount of a single-stranded binding moiety may be used. For example, at least about 100 picograms (pg), such as at least about 100 pg, 1 nanogram (ng), 5 ng, 10 ng, 20 ng, 30 ng, 40 ng, 50 ng, 60 ng, 70 ng, 80 ng, 90 ng, 100 ng, 200 ng, 300 ng, 400 ng, 500 ng, 600 ng, 700 ng, 800 ng, 900 ng, 1 microgram (µg), 10 µg, 25 µg, 50 µg, 100 µg, 200 µg, 400 µg, 500 µg, or more, of a single-stranded binding moiety (e.g., SSB protein) may be used per 1 million particles. For example, the concentration of single-stranded binding moiety may be at a concentration between about 100 pg single-stranded binding moiety/million particles and about 500 µg single-stranded binding moiety/million particles. For example, the concentration of single-stranded binding moiety may be at a concentration between about 100 ng single-stranded binding moiety/million particles and about 400 ng single-stranded binding moiety/million particles. Alternatively, the concentration of single-stranded binding moiety may be no more than a concentration selected between about 100 pg single-stranded binding moiety/million particles and 500 µg single-stranded binding moiety/million particles. For example, at least about 50 ng of an SSB protein may be used for a million particles, such as at least about 100 ng, 200 ng, 300 ng, 400 ng, 500 ng, or more SSB protein. Similarly, any useful concentration of a single-stranded binding moiety may be used. For example, at least about 0.01 µM of a single-stranded binding moiety may be used, such as at least about 1.0 nM, 2.0 nM, 5.0 nM, 10.0 nM, 20.0 nM, 50.0 nM, 100.0 nM, 0.2 µM, 0.3 µM, 0.4 µM, 0.5 µM, 0.75 µM, 1.0 µM, 1.1 µM, 1.25 µM, 1.5 µM, 1.75 µM, 2.0 µM, 2.5 µM, 3.0 µM, 4.0 µM, 5.0 µM, 6.0 µM, 7.0 µM, 8.0 µM, 9.0 µM, 10.0 µM, 12.0 µM, 14.0 µM, 16.0 µM, 18.0 µM, 20.0 µM, 50.0 µM, 100.0 µM, 200.0 µM, 250.0 µM, 300.0 µM, 350.0 µM,400.0 µM, 450.0 µM,500.0 µM,600.0 µM,700.0 µM,800.0 µM,900.0 µM,1 millimolar (mM), or more. Any useful volume of a solution comprising a single-stranded binding moiety may be used. For example, at least about 1 picoliter (pL) may be used, such as at least about 2 pL, 5 pL, 10 pL, 20 pL, 50 pL, 100 pL, 200 pL, 500 pL, 1 nanoliter (nL), 10 nL, 20 nL, 50 nL, 100 nL, 200 nL, 500 nL, 1 microliter (µL), 10 µL, 20 µL, 50 µL, 100 µL, 200 µL, 500 µL, 1 milliliter (mL), or more may be used. The amount of a single-stranded binding moiety needed to effectively reduce particle aggregation and/or otherwise improve sequencing quality may be impacted by the use of reagents such as salt (e.g., magnesium salt such as MgC1₂), spermine or spermidine, and cobalt hexammine, which may increase the footprint of an SSB protein from about 30 bases to about 60 bases per tetramer. Other factors including, for example, temperature, pH, and ionic strength may also impact aggregation of particles comprising nucleic acid molecules.

Contacting a nucleic acid molecule (e.g., a nucleic acid molecule coupled to a particle) with a binding moiety (e.g., a single-stranded binding moiety) may comprise bringing the nucleic acid molecule and the binding moiety into physical proximity to one another. Contacting a nucleic acid molecule with a binding moiety may comprise providing a reaction mixture comprising the binding moiety, which reaction mixture may comprise one or more reagents. For example, the reaction mixture may comprise a salt. A reaction mixture may comprise a polyamine such as spermidine and/or another stabilizing cation such as cobalt hexammine. Upon contacting a nucleic acid molecule and a binding moiety, a treated particle may be provided. The treated particle may comprise the nucleic acid molecule having a single-stranded region that is associated with the binding moiety. This process may be repeated, e.g., substantially simultaneously or sequentially, for a plurality of nucleic acid molecules coupled to a given particle and a plurality of binding moieties (e.g., single-stranded binding moieties, such as single-strand binding proteins). Similarly, the process may be repeated, e.g., substantially simultaneously or sequentially, for a plurality of particles comprising a plurality of nucleic acid molecules and a plurality of binding moieties (e.g., single-stranded binding moieties, such as single-strand binding proteins). Nucleic acid molecules of different particles of the plurality of particles may have sequence identity to one another. Alternatively or additionally, nucleic acid molecules of different particles of the plurality of particles may not have sequence identity to one another. For example, a sequence of a nucleic acid molecule coupled to a first particle may have less than 99% sequence complementarity to a sequence of a nucleic acid molecule coupled to a second particle, such as less than 95%, 90%, 85%, 80%, 75%, 70%, 65%, 60%, 55%, 50%, 45%, 40%, or less sequence complementarity. The plurality of binding moieties (e.g., single-stranded binding moieties) may be of a single type or a plurality of different types. In some cases, at least 50% of the nucleic acid molecules of a plurality of nucleic acid molecules coupled to a given particle, such as at least 55%, 60%, 65%, 70%, 75%, 80%, 85%, 90%, 95%, 98%, 99%, or more, may be associated with (e.g., bound to) a binding moiety of a plurality of binding moieties. In certain cases, at least 70% of the nucleic acid molecules of a plurality of nucleic acid molecules coupled to a given particle may be associated with (e.g., bound to) a binding moiety of a plurality of binding moieties. In some cases, at least 90% of the nucleic acid molecules of a plurality of nucleic acid molecules coupled to a given particle may be associated with (e.g., bound to) a binding moiety of a plurality of binding moieties. Where more than one particle is used, nucleic acid molecules of at least 50% of a plurality of particles may be associated with (e.g., bound to) binding moieties of a plurality of binding moieties. In some cases, nucleic acid molecules of at least 70% of a plurality of particles may be associated with (e.g., bound to) binding moieties of a plurality of binding moieties. In some cases, nucleic acid molecules of at least 90% of a plurality of particles may be associated with (e.g., bound to) binding moieties of a plurality of binding moieties. Where a particle comprises a first plurality of nucleic acid molecules having a first sequence and a second plurality of nucleic acid molecules having a second sequence different than the first sequence, different concentrations of first and second nucleic acid molecules may be coupled to binding moieties. For example, a first plurality of binding moieties may be configured to bind to nucleic acid molecules of the first plurality of nucleic acid molecules and binding moieties of a second plurality of binding moieties may be configured to bind to nucleic acid molecules of the second plurality of nucleic acid molecules. The first plurality of binding moieties may be of a first type and the second plurality of binding moieties may be of a second type. Alternatively, the first plurality of binding moieties may be a first plurality of nucleic acid molecules and the second plurality of binding moieties may be a second plurality of nucleic acid molecules, where the first plurality of nucleic acid molecules and the second plurality of nucleic acid molecules comprise different sequences and are configured to bind to different target sequences. The population of binding moieties used in a treating process may be varied to ensure that desired target sequences coupled to one or more different particles in a solution are contacted with appropriate binding moieties. For example, one or more different types of binding moieties may be provided in excess, and/or a concentration control process may be used to control the number of nucleic acid molecules having given sequences that are contacted with appropriate binding moieties.

In an example, a first particle coupled to a nucleic acid molecule and a second particle may be used in a method of processing a plurality of particles comprising the first and second particles. The first particle and the second particle may have the same or different composition, size, shape, and other features. For example, the first particle and the second particle may be of approximately the same size and shape and may have similar compositions. The first particle may comprise a plurality of nucleic acid molecules coupled (e.g., immobilized) thereto, where the plurality of nucleic acid molecules comprises the nucleic acid molecule, and where at least a subset of the plurality of nucleic acid molecules have sequence identity to a first nucleic acid sequence. The second particle may comprise an additional plurality of nucleic acid molecules coupled (e.g., immobilized) thereto, where the additional plurality of nucleic acid molecules is different from the plurality of nucleic acid molecules and where at least a subset of the additional plurality of nucleic acid molecules have sequence identity to a second nucleic acid sequence. The second nucleic acid sequence may have a different nucleic acid sequence than the first nucleic acid sequence. Alternatively, the second nucleic acid sequence may have sequence identity to the first nucleic acid sequence. The plurality of nucleic acid molecules coupled to the first particle may comprise at least 100 nucleic acid molecules, such as at least 500, 1,000, 2,000, or more nucleic acid molecules. Similarly, the additional plurality of nucleic acid molecules coupled to the second particle may comprise at least 100 nucleic acid molecules, such as at least 500, 1,000, 2,000, or more nucleic acid molecules. Nucleic acid molecules of the plurality of nucleic acid molecules and additional nucleic acid molecules of the additional plurality of nucleic acid molecules may share one or more nucleic acid sequences (e.g., functional sequences, as described herein) and/or may be coupled to their respective particles via the same or different mechanisms. The first particle may be contacted with a plurality of binding moieties (e.g., single-stranded binding moieties, such as single-strand binding proteins) under conditions sufficient to couple binding moieties of the plurality of binding moieties to nucleic acid molecules of the plurality of nucleic acid molecules of the first particle to provide a plurality of treated nucleic acid molecules coupled to the first particle. This process may be repeated for the second particle (e.g., with a second plurality of binding moieties). Alternatively, the second particle may not be treated in this manner.

The first particle comprising the plurality of treated nucleic acid molecules may be provided in a solution comprising the second particle. The first particle and second particle may be included in a same solution while the first particle is contacted with the plurality of binding moieties. Alternatively, the first particle and second particle may not be included in a same solution while the first particle is contacted with the plurality of binding moieties. For example, the first particle may be included in a first solution and the second particle may be included in a second solution. In an example, the first particle may be included in a partition (e.g., a droplet or a well, such as a droplet of an aqueous emulsion or a well of a microwell plate) prior to being provided in a same solution with the second particle. The first particle may be contacted with the plurality of binding moieties in a partition. Alternatively, the first particle may be contacted with the plurality of binding moieties outside of a partition. A solution comprising the first particle and the second particle may be an aqueous solution. The solution may comprise one or more reagents for storing and/or processing nucleic acid molecules. For example, the solution may comprise a cryoprotective agent to facilitate storage of the solution at a low temperature, such as in a freezer. Alternatively or additionally, the solution may comprise a stabilizing agent to facilitate storage of the solution, optionally at room temperature. The solution may be provided in a sealed container, such as a sealed tube or flask. Such a container may be air- and/or watertight. The solution may be stored for at least 1 hour, such as least 2 hours, 4 hours, 8 hours, 10 hours, 12 hours, 18 hours, 24 hours, 48 hours, 72 hours, or longer. For example, the solution may be stored for at least 1 day, 2 days, 4 days, 7 days, 2 weeks, 3 weeks, 4 weeks, 1 month, 2 months, 3 months, 6 months, 12 months, or longer. The solution may be stored during transit between a first location and a second location. For example, the solution may be stored during transit between a first location where the treated particle is prepared to a second location where downstream processing (e.g., nucleic acid amplification and/or sequencing) may be performed.

The methods, systems, compositions, and kits provided herein may be useful for reducing particle aggregation and contamination for substrates comprising nucleic acid molecules. For example, the methods, systems, compositions, and kits provided herein may be useful for preventing contaminant nucleic acid molecules from interacting with (e.g., hybridizing to) nucleic acid molecules coupled to substrates such as particles (e.g., beads) and/or particles immobilized to a support. Similarly, the methods, systems, compositions, and kits provided herein may be useful for preventing interactions between nucleic acid molecules including nucleic acid molecules coupled to substrates such as particles (e.g., beads) and/or particles immobilized to a support. For example, interactions between nucleic acid molecules coupled to a given substrate may be reduced by contacting one or more of the nucleic acid molecules with a binding moiety configured to bind to a sequence of the one or more nucleic acid molecules. Interactions between nucleic acid molecules coupled to different substrates (e.g., different particles, including different particles immobilized to a support) may be reduced in a similar manner. Accordingly, the methods, systems, compositions, and kits provided herein may be useful for preventing aggregation between one or more particles and or between one or more particles and a support. Such methods, systems, compositions, and kits may be implemented at various points during a processing involving a substrate comprising a nucleic acid molecule coupled thereto. For example, aggregation and/or contamination may be prevented and/or mediated as provided herein upon generation of the substrate comprising a nucleic acid molecule coupled thereto, such as upon manufacturing a particle comprising a nucleic acid molecule coupled thereto. The particle may then be stored in a solution with one or more additional particles and/or a support. Aggregation and/or contamination may also be prevented or mediated as provided herein during processing of a nucleic acid molecule coupled to a particle. For example, a nucleic acid molecule coupled to a particle may comprise a capture sequence coupled thereto that is configured to capture a template nucleic acid sequence. Upon capture of the template nucleic acid sequence (e.g., via a ligation, hybridization, splint-based hybridization, or other mechanism), the template nucleic acid sequence may be optionally amplified and/or otherwise processed in preparation for subsequent downstream analysis via, e.g., a nucleic acid sequencing assay. The methods, systems, compositions, and kits provided herein may be used to facilitate template attachment to the particle, and/or to facilitate storing and subsequent processing of the template-attached particle after the attachment process.

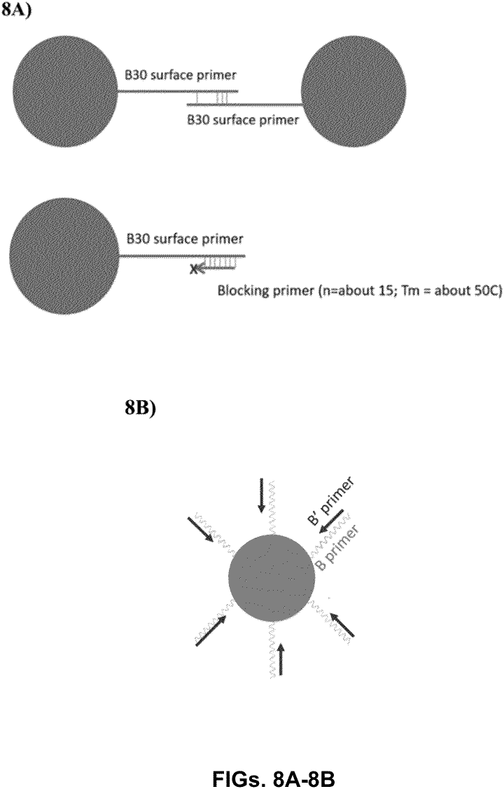

Treating particles according to the methods provided herein may allow for the preparation of more stable compositions including fewer particle aggregates than compositions including particles not treated according to such methods. For example, the methods, systems, kits, and compositions provided herein may prevent formation of particle aggregates based on hybridization of like-sequences on separate particles. Such methods, systems, kits, and compositions may be useful using particles at pre-amplification states, where separate particles comprise like sequences in, e.g., surface primer molecules that can, or tend to, hybridize with each other, and also useful using particles at post-amplification states, where separate particles comprise like sequences in, e.g., amplified products such as DNA inserts immobilized to the particles that can, or tend to, hybridize with each other. For example, the upper panel of FIG. 8A shows formation of an aggregate including particles each comprising a nucleic acid molecule comprising a common nucleic acid sequence (e.g., a B30 surface primer sequence). The common nucleic acid sequences include multiple points of overlap, which may be common for human genomic material, for example. In the lower panel of FIG. 8A, a single-stranded binding moiety, shown here as a blocking primer molecule, is used to block access to the free single-stranded region of the nucleic acid molecule coupled to a particle, making it unavailable for hybridization to another nucleic acid molecule coupled to a separate particle and thus preventing aggregate formation. A random N-mer (e.g., a random hexamer) or other single-stranded binding moiety such as a single-strand binding protein may be used in place of a blocking primer molecule. FIG. 8B shows a particle comprising nucleic acid molecules coupled thereto, each of which comprises a B primer sequence, where each nucleic acid molecule is blocked with a nucleic acid molecule comprising a sequence complementary to the sequence of the B primer (e.g., a B′ primer). Aggregation of particles may be assessed using, for example, visualization methods including microscopy (e.g., electron microscopy or fluorescence microscopy) and nucleic acid sequencing. Reduction of particle aggregates and/or the size of particle aggregates (e.g., clumps) may impact loading of particles onto a substrate (e.g., wafer) for subsequent analysis (e.g., nucleic acid sequencing) as well as the proportion of “live” particles (e.g., particles available for sequencing) available upon loading of a substrate. Accordingly, reducing particle aggregates may improve cost efficiency as well as downstream sequencing quality.

A blocking oligonucleotide, such as a blocking primer molecule, may have any useful structure, size, and composition (e.g., as described herein). A blocking oligonucleotide may be linear, as illustrated in FIGS. 8A and 8B. Alternatively, a blocking oligonucleotide may include one or more branchpoints, loops, secondary structures, or any other structural features. For example, a blocking oligonucleotide may include a loop or a hairpin-like feature. FIGS. 8A and 8B illustrate blocking primers bound directly to surface primers B30; for example, prior to attachment of target nucleic acid molecules to the particles. One or more types of single strand binding moieties may be added at any point of the process. As an example, and without limitation, a single strand binding moiety can be added, alternatively or additionally, to particles (e.g., beads) after target nucleic acid molecules are attached to the particles, after target nucleic acid molecules are amplified but before sequencing, and/or during a sequencing process.

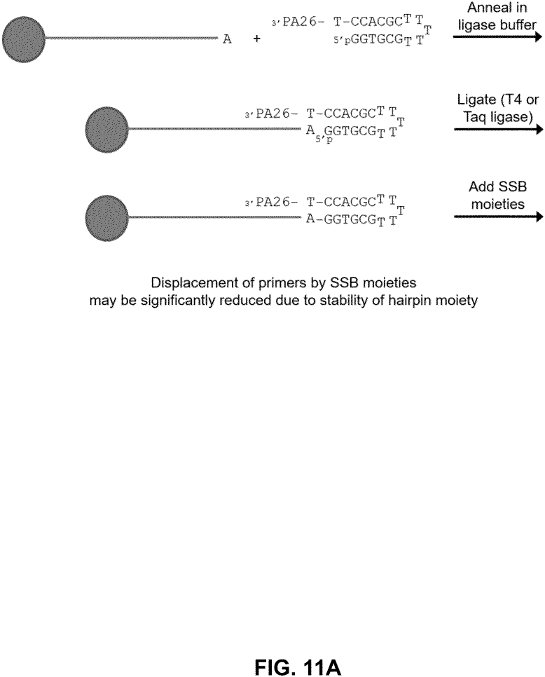

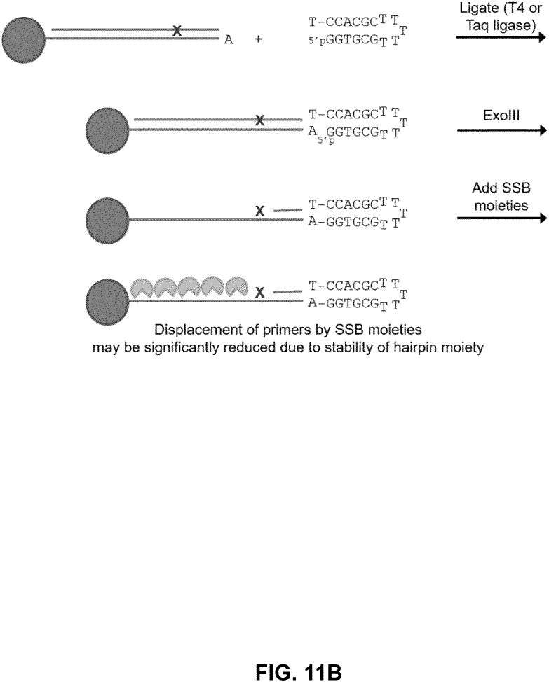

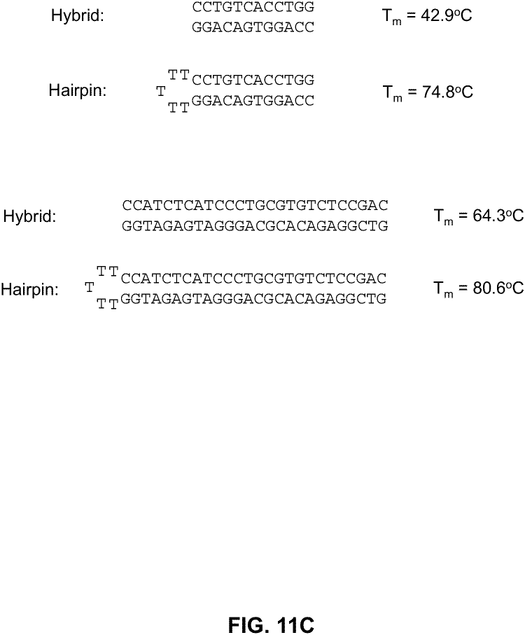

In some cases, a single strand binding moiety (e.g., a single stranded binding protein) is added during the sequencing process to reduce secondary or tertiary structures in target nucleic acid molecules. When the single strand binding moiety reaches a certain concentration, it can also interfere with the binding interactions between sequencing primers and the target nucleic acid molecules. As disclosed herein, a number of methods can be applied to enhance the binding interactions between sequencing primers and the target nucleic acid molecules (e.g., as indicated by higher melting temperatures of the sequencing primer-target nucleic acid molecule duplex). In some cases, longer sequencing primers can be used. For example, sequencing primers may include at least 30, 32 34, 35, 36, 38, 40, 42, 44, 45, or even more nucleic acids. In some embodiments, locked nucleic acid molecules (LNAs, also known as inaccessible RNAs) can be used. For example, LNAs may comprise a modified RNA nucleotide in which the ribose moiety is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in an oligonucleotide and hybridize with DNA or RNA according to Watson-Crick base-pairing rules. The locked ribose conformation may enhance base stacking and backbone pre-organization. This can significantly increase the hybridization properties (e.g., melting temperature) of oligonucleotides. In some cases, a sequencing primer can include a hairpin moiety. For example, the hairpin moiety can wrap around the target nucleic acid molecule, placing the terminal residue close to a terminal adenosine-containing nucleotide (A-tail), and be ligated to the terminal adenosine-containing nucleotide, thereby converting the original duplex intermolecular binding interactions to intramolecular interactions. Details of this example are illustrated in FIGS. 11A-11C.

FIG. 11A shows an example schematic in which a sequencing primer (e.g., PA26) is extended to include a loop feature (e.g., a hairpin moiety). The upper panel of FIG. 11A shows a nucleic acid molecule attached (e.g., covalently attached) to a particle (e.g., a bead) (e.g., as described herein). In some cases, the nucleic acid molecule includes a terminal adenine-containing nucleotide, which nucleotide may be included in an adapter sequence and/or may have been added to the nucleic acid molecule in an A-tailing process (e.g., when using a Taq polymerase) or in an amplification process using a terminal transferase. The nucleic acid molecule may have any useful length, features, and composition (e.g., as described herein). The nucleic acid molecule may include one or more primer or adapter sequences. The nucleic acid molecule may include a sequence of a template nucleic acid, or a complement thereof. For example, the nucleic acid can include an amplified copy of a target nucleic acid molecule that is covalently attached to the particle via a surface primer. The nucleic acid molecule may include functional sequences including a spacer sequence, an index or barcode sequence, a unique identifier sequence, a sequencing primer sequence, a flow cell adapter sequence, or any other useful sequence (e.g., as described herein). The nucleic acid molecule may have been prepared in solution and subsequently attached to the particle. Alternatively, the nucleic acid molecule may have been prepared while attached to the particle. For example, the nucleic acid molecule may have been prepared via an amplification process, e.g., using a primer sequence coupled (e.g., covalently attached) to the particle. Accordingly, the nucleic acid molecule may be an extended primer molecule.

The nucleic acid molecule coupled to the particle shown in FIG. 11A may include a primer sequence or a sequence complementary to a specific primer sequence. For example, the illustrated nucleic acid molecule may include a sequence complementary to a primer sequence (e.g., a “PA26” primer sequence) distal to the particle. The sequence complementary to the primer sequence may be adjacent to the terminal A nucleotide. Alternatively, the sequence complementary to the primer sequence may be separated from the terminal A nucleotide by one or more additional nucleotides. The nucleic acid molecule may be contacted with a molecule including a hairpin moiety, as shown in the upper panel. The molecule including the hairpin moiety includes a primer sequence (e.g., a PA26 primer sequence). The primer sequence may be adjacent to a T-containing nucleotide. Alternatively, the primer sequence may be separated from the T-containing nucleotide by one or more nucleotides. The molecule including the hairpin molecule may include a double-stranded portion, which double-stranded portion may have any useful length (e.g., at least 1, 2, 3, 4, 5, 6, 7, 8, 9, 10, or more base pairs). The double-stranded portion may include at least 1 C-G base pair, such as at least 2, 3, 4, 5, or more C-G base pairs. The loop structure of the hairpin moiety may have any useful structure. For example, the loop structure may include a single type of nucleotide (e.g., a single type of canonical nucleotide, such as T-containing nucleotides). Alternatively, the loop structure may include two or more different types of nucleotides. The loop structure of the hairpin moiety may include any useful number of nucleotides, such as 3, 4, 5, 6, 7, or more nucleotides. For example, the loop structure of the hairpin moiety may include 5 nucleotides. In FIG. 11A, the loop structure includes 5 T-containing nucleotides. The nucleic acid molecule coupled to the particle may be contacted with the molecule including the hairpin moiety under conditions sufficient to anneal the primer sequence in the molecule including the hairpin moiety to the complementary sequence in the nucleic acid molecule coupled to the particle. Such conditions may comprise a ligase buffer. As shown in the middle panel of FIG. 11A, the primer sequence of the molecule including the hairpin moiety may hybridize to the complementary sequence in the nucleic acid molecule coupled to the particle. The terminal A-containing nucleotide of the nucleic acid molecule coupled to the particle may also hybridize to a T-containing nucleotide of the molecule including the hairpin moiety. The hybrid structure may be subjected to conditions sufficient to ligate the terminal A-containing nucleotide of the nucleic acid molecule coupled to the particle to the terminal nucleotide of the molecule including the hairpin moiety, shown in FIG. 11A as a G-containing nucleotide. Such conditions may comprise, e.g., a ligase such as a T4 or Taq ligase. The resultant structure includes an intramolecular hairpin moiety and may have enhanced stability compared to a nucleic acid molecule coupled to the primer sequence in the absence of the hairpin moiety. Accordingly, upon contacting the nucleic acid molecule coupled to the particle with SSB moieties (e.g., as described herein), such as SSB proteins, the SSB moieties may be less likely to displace the primer sequence hybridized to the nucleic acid molecule coupled to the particle than they might be in the absence of the hairpin moiety.

Though FIG. 11A shows addition of a hairpin moiety to a single-stranded nucleic acid molecule coupled to the particle, a hairpin moiety may also be incorporated into a system including a double-stranded nucleic acid molecule coupled to a particle (e.g., bead), as shown in FIG. 11B. The upper panel of FIG. 11B shows a double-stranded nucleic acid molecule coupled to a particle (e.g., bead). The nucleic acid molecule may have any useful length, features, and composition (e.g., as described herein). The nucleic acid molecule may include one or more single-stranded portions. The nucleic acid molecule may comprise a sequence of a template nucleic acid molecule (e.g., derived from a nucleic acid sample) in one or both strands. The nucleic acid molecule may include functional sequences including a spacer sequence, an index or barcode sequence, a unique identifier sequence, a sequencing primer sequence, a flow cell adapter sequence, or any other useful sequence (e.g., as described herein), which sequences may be included in either strand. The nucleic acid molecule may include one or more primer or adapter sequences. For example, the double-stranded nucleic acid molecule may include a primer sequence in a first strand and a complement of the primer sequence in a second strand. The first strand may be directly coupled (e.g., covalently attached) to the particle. Alternatively, the second strand may be directly coupled (e.g., covalently attached) to the particle. The nucleic acid molecule may have been prepared in solution and subsequently attached to the particle. Alternatively, the nucleic acid molecule may have been prepared while attached to the particle. For example, the nucleic acid molecule may have been prepared via an amplification process, e.g., using a primer sequence coupled (e.g., covalently attached) to the particle. For example, the nucleic acid molecule may have been prepared via an amplification process using a primer sequence included in a first strand of the nucleic acid molecule coupled (e.g., covalently attached) to the particle. The first strand may comprise an extended primer sequence, which extended primer sequence may have been used to prepare the double-stranded nucleic acid molecule. The illustrated nucleic acid molecule in FIG. 11B includes a terminal adenine-containing nucleotide, which nucleotide may be included in an adapter sequence and/or may have been added to the nucleic acid molecule in an A-tailing process (e.g., using a Taq polymerase) or in an amplification process using a terminal transferase. The terminal A-containing nucleotide may be included in a strand that is covalently attached to the particle. Alternatively, the terminal A-containing nucleotide may be included in a strand that is not covalently attached to the particle. The illustrated nucleic acid molecule may also include a reactive site. The reactive site of the nucleic acid molecule shown in FIG. 11B is indicated by an “X” symbol. The reactive site may comprise a cleavable or excisable moiety, such as an uracil base. Alternatively, the reactive site may comprise a digestible moiety. For example, the reactive site may comprise a phosphorothioate moiety.

The nucleic acid molecule coupled to the particle may be contacted with a molecule comprising a hairpin moiety. The molecule including the hairpin molecule may include a double-stranded portion, which double-stranded portion may have any useful length (e.g., at least 1, 2, 3, 4, 5, 6, 7, 8, 9, 10, or more base pairs). The double-stranded portion may include at least 1 C-G base pair, such as at least 2, 3, 4, 5, or more C-G base pairs. The loop structure of the hairpin moiety may have any useful structure. For example, the loop structure may include a single type of nucleotide (e.g., a single type of canonical nucleotide, such as T-containing nucleotides). Alternatively, the loop structure may include two or more different types of nucleotides. The loop structure of the hairpin moiety may include any useful number of nucleotides, such as 3, 4, 5, 6, 7, or more nucleotides. For example, the loop structure of the hairpin moiety may include 5 nucleotides. In FIG. 11B, the loop structure includes 5 T-containing nucleotides. The nucleic acid molecule coupled to the particle may be contacted with the molecule comprising the hairpin moiety under conditions sufficient to hybridize a portion of the molecule comprising the hairpin moiety to the nucleic acid molecule. For example, a terminal T-containing nucleotide of the molecule comprising the hairpin moiety may hybridize to a terminal A-containing nucleotide of the nucleic acid molecule coupled to the particle. In some cases, a strand of the nucleic acid molecule coupled to the particle may comprise two or more nucleotides to which the molecule comprising the hairpin moiety may hybridize. Hybridization between these moieties may serve to stabilize the double-stranded nucleic acid molecule (e.g., relative to a nucleic acid molecule that does not include a hairpin moiety). A ligase (e.g., T4 or Taq ligase) may be used to hybridize the terminal A-containing nucleotide of the nucleic acid molecule coupled to the particle to a terminal nucleotide of the molecule comprising the hairpin moiety (shown in FIG. 11B as a G-containing nucleotide). An additional enzyme such as an exonuclease may be used to remove a portion of the double-stranded nucleic acid molecule. For example, an exonuclease (e.g., ExoIII) may be used to remove the portion of a strand of the double-stranded nucleic acid molecule downstream of the reactive site, as shown in the third panel of FIG. 11B. Where a primer sequence was included in the partially cleaved strand (e.g., adjacent to the reactive site), the resultant structure may include a primer sequence for use in a later amplification and/or sequencing process. Accordingly, the resultant structure may include an intramolecular hairpin moiety that may have enhanced stability compared to a nucleic acid molecule coupled to the primer sequence in the absence of the hairpin moiety. Thus, upon contacting the nucleic acid molecule coupled to the particle with SSB moieties (e.g., as described herein), such as SSB proteins, the SSB moieties may be less likely to displace the primer sequence hybridized to the nucleic acid molecule coupled to the particle than they might be in the absence of the hairpin moiety.

FIG. 11C illustrates the stability of double-stranded nucleic acid molecules relative to the same double-stranded nucleic acid molecules including an appended loop structure. As shown in the upper panel, the illustrated short, C-G rich sequence has a melting temperature, Tₘ, of 42.9° C., while the same sequence with an appended loop structure has a melting temperature of 74.8° C. Similarly, the longer sequence shown in the lower panel has a melting temperature of 64.3° C., while the same sequence with an appended loop structure has a melting temperature of 80.6° C. Accordingly, inclusion of a hairpin moiety in a structure including a primer sequence hybridized to a nucleic acid molecule may serve to stabilize the primer sequence, thereby reducing denaturation and/or displacement by single-stranded binding moieties.

A particle comprising a treated nucleic acid molecule (e.g., a treated particle) may be immobilized to a substrate (e.g., as described herein). A substrate may be a disk, such as a rotatable disk. A substrate may be patterned. For example, a substrate may comprise a plurality of areas comprising a chemical feature (e.g., capture moiety) and a plurality of areas that do not comprise the chemical feature. A substrate may comprise a plurality of areas that are raised or depressed relative to other areas of the substrate, such as a plurality of wells. A substrate may comprise a plurality of independently addressable locations. A substrate may be substantially planar. For example, a plurality of independently addressable locations of a substrate may be substantially planar. A plurality of independently addressable locations of a substrate may comprise one or more wells. A substrate may comprise a solution comprising a treated particle and, in some cases, an additional particle (e.g., a first particle and second particle). A substrate may be positioned in contact with a treated particle and, in some cases, an additional particle. In an example, a particle may be treated as described herein and provided in solution with an additional particle. The solution comprising the treated particle and the additional particle may then be brought into contact with a substrate, at which time the treated particle may be immobilized to the substrate (e.g., via a capture moiety). In another example, a particle may be immobilized to a substrate. The immobilized particle may be treated as described herein and an additional particle may be provided, such that the immobilized particle and the additional particle are included in a same solution.

A particle comprising a treated nucleic acid molecule may undergo additional processing. For example, non-treated nucleic acid molecules coupled to the particle may be removed from the particle and/or completely or partially digested such that the treated particle comprises only the treated nucleic acid molecule (or, where a plurality of treated nucleic acid molecules are present, the plurality of treated nucleic acid molecules). The particle may be subjected to storage at room temperature or a freezing temperature (e.g., as described herein). The particle may be immobilized to a substrate (e.g., as described herein). In some cases, a solution comprising a plurality of particles comprising a treated particle may be subjected to filtration, centrifugation, agitation, or another mechanical process, such as to remove particles that may have aggregated together via associations between non-treated nucleic acid molecules. In some cases, magnetic sorting may be used to separate a treated particle from other particles in a solution. For example, a particle may comprise a magnetic moiety (e.g., coupled to a single-stranded binding moiety associated with a nucleic acid molecule of the particle), which magnetic moiety may be used to separate the particle from non-treated particles. In some cases, an affinity moiety may be used to separate a treated particle from other particles in a solution. For example, a particle may comprise an affinity moiety (e.g., coupled to a single-stranded binding moiety associated with a nucleic acid molecule of the particle), which affinity moiety may be used to separate the particle from non-treated particles. Such a method may comprise the use of a biotin-avidin (e.g., streptavidin) association. In some cases, a treated particle may be subjected to optical detection subsequent to an isolation, separation, or enrichment process. For example, an optically detectable moiety of the treated particle and/or a single-stranded binding moiety coupled thereto may be detected. Such a process may be used to identify treated particles comprising nucleic acid molecules coupled to binding moieties.

A treated particle (e.g., in a solution and/or immobilized to a substrate) may be optically detected. For example, the treated particle may be optically detectable and/or may comprise an optically detectable moiety, such as a fluorescent moiety. In an example, a solution comprises a plurality of particles, each of which is optically detectable. A first subset of the plurality of particles comprises treated particles, while a second subset of the plurality of particles comprises non-treated particles. Imaging of the plurality of particles of the solution may reveal aggregation of non-treated particles, and/or may facilitate separation of treated particles from non-treated particles. The plurality of particles may be immobilized to a substrate (e.g., as described herein). An optically detectable moiety may be coupled to a surface of a particle (e.g., a treated particle). For example, an optically detectable moiety may be coupled to a nucleic acid molecule coupled to a particle. Alternatively or additionally, an optically detectable moiety may be included within and/or distributed throughout a particle. For example, an optically detectable moiety may be coupled to a polymer network of a particle.

A treated nucleic acid molecule of a particle (e.g., a nucleic acid molecule comprising a single-stranded region associated with a single-stranded binding moiety, such as a single-strand binding protein) may be used in processing of a template (e.g., sample) nucleic acid molecule. For example, a single-stranded binding moiety associated with a single-stranded region of the treated nucleic acid molecule may be disassociated from the single-stranded region and the single-stranded region may be undergo a nucleic acid reaction with the template nucleic acid molecule. The template nucleic acid molecule may derive from a sample (e.g., as described herein) such that the template nucleic acid molecule comprises a sequence corresponding to the sample. The template nucleic acid molecule may comprise an adapter sequence at one or both ends.

The template nucleic acid molecule may comprise a sequence (e.g., in an adapter coupled to the template nucleic acid molecule) complementary to a sequence of the single-stranded region such that the sequences may hybridize to one another. Subsequent primer extension may be used to generate a copy of a sequence of the template nucleic acid molecule. In some cases, such a process may be used to generate one or more copies of sequences of the template nucleic acid molecules. Amplification of the template nucleic acid molecule may be used to prepare a clonal population of nucleic acid molecules comprising sequences of the template nucleic acid molecule or sequences complementary thereto. Alternatively or additionally, a nucleic acid sequencing reaction (e.g., sequencing by synthesis) may be performed. A sequencing process may comprise sequencing all or a portion of the single-stranded region of the nucleic acid molecule. A sequencing process may comprise the use of terminated and/or non-terminated nucleotides. For example, a sequencing process may comprise the use of non-terminated nucleotides such that homopolymeric regions of the single-stranded region of the nucleic acid molecule and/or of the template nucleic acid molecule may be sequenced. Nucleotides and/or nucleotide analogs used in a sequencing process may comprise detectable labels (e.g., fluorescent dyes), which detectable labels may be linked to the nucleotides and/or nucleotide analogs via linkers (e.g., semi-rigid linkers). A sequencing process may comprise any useful method, including, for example, sequencing by synthesis, sequencing by ligation, sequencing by hybridization, nanopore sequencing, single molecule sequencing, and pyrosequencing.

A treated particle may comprise a single treated nucleic acid molecule or a finite number of treated nucleic acid molecules. For example, the particle may initially be prepared with a single primer nucleic acid molecule coupled thereto such that a single template nucleic acid molecule may be coupled to the particle. Alternatively, concentration control methods may be utilized to ensure that a limited number of template nucleic acid molecules may be coupled to a particle prior to it undergoing stabilizing treatment as described herein. A particle may be treated subsequent to a template nucleic acid molecule hybridizing to a primer nucleic acid molecule coupled thereto. A primer extension process may also take place prior to treatment of the particle. In this regard, the reduction of particle aggregation facilitated by the methods provided herein may be applied subsequent to an amplification process.

In an aspect, the present disclosure provides a system comprising a first solution comprising a suspension comprising a plurality of particles (e.g., beads) comprising a first particle and a second particle. The plurality of particles may comprise, for example, at least about 1,000 particles (e.g., at least about 500, 1,000, 1,500, 2,000, 2,500, 3,000, 5,000, 10,000, 50,000, 100,000, 500,000, 1,000,000, 2,000,000, 5,000,000, 10,000,000, or more particles). For example, the plurality of particles may comprise at least about 100,000 particles, such as at least about 1,000,000 particles. A “suspension,” as used herein, may comprise a liquid phase comprising free-floating and/or mobile solid phases. The plurality of particles may comprise a plurality of nucleic acid molecules immobilized thereto, such that the first particle comprises a first subset of the plurality of nucleic acid molecules immobilized thereto, which first subset of the plurality of nucleic acid molecules comprises a first nucleic acid molecule comprising a single-stranded portion. The first subset of the plurality of nucleic acid molecules may have at least partial sequence identity to a first nucleic acid sequence. The first nucleic acid sequence may comprise a sequence of a sample (e.g., template) nucleic acid molecule. The first subset of the plurality of nucleic acid molecules may comprise, for example, at least about 1,000 nucleic acid molecules (e.g., at least about 500, 1,000, 1,500, 2,000, 2,500, 3,000, 5,000, 10,000, 50,000, 100,000, 200,000, 500,000, 1,000,000, or more nucleic acid molecules). The second particle may comprise a second subset of the plurality of nucleic acid molecules immobilized thereto. The second subset may be different from the first subset, and the second subset of the plurality of nucleic acid molecules may have at least partial sequence identity to a second nucleic acid sequence different from the first nucleic acid sequence. The second subset may comprise, for example, at least about 1,000 nucleic acid molecules (e.g., at least about 500, 1,000, 1,500, 2,000, 2,500, 3,000, 5,000, 10,000, 50,000, 100,000, 200,000, 500,000, 1,000,000, or more nucleic acid molecules). The system may further comprise a second solution comprising a single-stranded binding moiety configured to couple to the single-stranded portion of the first nucleic acid molecule. The plurality of nucleic acid molecules may comprise a plurality of DNA molecules, a plurality of RNA molecules or a combination thereof. The single-stranded portion of the first nucleic acid molecule may comprise single-stranded DNA (ssDNA) and/or RNA. The first nucleic acid molecule may comprise a priming sequence, or a complement thereof. Such a priming sequence may be, for example, a targeted priming sequence or a random N-mer sequence. The first nucleic acid molecule may comprise a barcode sequence or a unique molecular identifier sequence, or any other useful sequence described herein. The second solution may comprise a plurality of single-stranded binding moieties, which plurality of single-stranded binding moieties may be of a single type. Alternatively, the plurality of single-stranded binding moieties may comprise a plurality of different types of single-stranded binding moieties. The second solution may further comprise one or more reagents configured to facilitate coupling of the single-stranded binding moiety and the single-stranded portion of the nucleic acid molecule. Such reagents may comprise salt, spermidine, and cobalt hexammine. Alternatively, such reagents may be separately provided or may be included in the first solution. The single-stranded binding moiety of the second solution may comprise a single-stranded binding (SSB) protein (e.g., as described herein), such as an SSB derived from E. coli or phage T4 (e.g., a T4 Gene 32 Protein or T4 SSB protein) or an Extreme Thermostable SSB protein. The single-stranded binding moiety may comprise an additional nucleic acid molecule, which additional nucleic acid molecule may comprise a random N-mer. A random N-mer may comprise, for example, between 6-10 nucleotides. In an example, the additional nucleic acid molecule of the single-stranded binding moiety comprises 6 or more nucleotides, such as 6, 7, 8, 9, or 10 nucleotides. Alternatively, the additional nucleic acid molecule may have sequence complementarity to a sequence of the single-stranded portion of the first nucleic acid molecule. The additional nucleic acid molecule may have any useful number of nucleotides, such as between 6-20 nucleotides.

In another aspect, the present disclosure provides a composition comprising a suspension comprising a plurality of particles (e.g., beads) comprising a first particle. The suspension may further comprise a second particle, such that the first particle is in fluidic communication with the second particle. The plurality of particles may comprise at least about 1,000 particles (e.g., at least about 500, 1,000, 1,500, 2,000, 2,500, 3,000, 5,000, 10,000, 50,000, 100,000, 1,000,000, 2,000,000, 5,000,000, 10,000,000, or more particles). For example, the plurality of particles may comprise at least 100,000 particles, such as at least about 1,000,000 particles. The plurality of particles may comprise a plurality of nucleic acid molecules immobilized thereto. The first particle may comprise a first nucleic acid molecule of the plurality of nucleic acid molecules immobilized thereto, and the second particle may comprise a second nucleic acid molecule of the plurality of nucleic acid molecules immobilized thereto. The first nucleic acid molecule may comprise a single-stranded portion. The second nucleic acid molecule may also comprise a single-stranded portion. The second nucleic acid molecule may have at least partial sequence identity to the first nucleic acid molecule. For example, the second nucleic acid molecule and the first nucleic acid molecule may both comprise a common nucleic acid sequence. Alternatively or additionally, the second nucleic acid molecule and the first nucleic acid molecule may comprise one or more different nucleic acid sequences. The first nucleic acid molecule and/or the second nucleic acid molecule may comprise a sequence of a sample (e.g., template) nucleic acid molecule. The plurality of nucleic acid molecules may comprise a plurality of DNA molecules, a plurality of RNA molecules or a combination thereof. The single-stranded portion of the first nucleic acid molecule may comprise single-stranded DNA (ssDNA) and/or RNA. The first nucleic acid molecule may comprise a priming sequence, or a complement thereof. Such a priming sequence may be, for example, a targeted priming sequence or a random N-mer sequence. The first nucleic acid molecule may comprise a barcode sequence or a unique molecular identifier sequence, or any other useful sequence described herein. The single-stranded portion of the first nucleic acid molecule may be associated with (e.g., bound to) a single-stranded binding moiety. The single-stranded binding moiety may comprise, for example, a single-stranded binding (SSB) protein, such as an SSB protein derived from E. coli or phage T4 (e.g., a T4 Gene 32 Protein or T4 SSB protein) or an Extreme Thermostable SSB protein. The single-stranded binding moiety may comprise an additional nucleic acid molecule, which additional nucleic acid molecule may comprise a random N-mer. A random N-mer may comprise, for example, between 6-10 nucleotides. In an example, the additional nucleic acid molecule of the single-stranded binding moiety comprises 6 or more nucleotides, such as 6, 7, 8, 9, or 10 nucleotides. Alternatively, the additional nucleic acid molecule may have sequence complementarity to a sequence of the single-stranded portion of the first nucleic acid molecule. The composition may comprise a plurality of single-stranded binding moieties comprising the single-stranded binding moieties. The plurality of single-stranded binding moieties may be of a same type. Alternatively, the plurality of single-stranded binding moieties may be of multiple types. At least 50%, such as at least 70% or 90%, of the plurality of particles may comprise immobilized thereto a nucleic acid molecule of the plurality of nucleic acid molecules that is coupled to a single-stranded binding moiety of a plurality of single-stranded binding moieties. In the composition, no more than 1%, such as no more than 0.1%, 0.01%, or 0.001%, of particles of the plurality of particles may be included in a particle aggregate (e.g., comprising two or more particles of the plurality of particles). Similarly, particle aggregates having a dimension (e.g., a cross-sectional dimension, such as a diameter) of at least 10 micrometers (µm), such as at least 1, 2, 3, 4, 5, 6, 7, 8, or 9 µm, may be absent from the composition. For example, particle aggregates having a dimension of at least 1 µm may be absent from the composition. Similarly, particle aggregates having an area (e.g., a cross-sectional area) of at least 10 µm², such as at least 8, 9, 10, 11, or 12 µm, may be absent from the composition. As described herein, an area of a particle aggregate may be assessed using an imaging detection method. The first particle may comprise a first subset of the plurality of nucleic acid molecules immobilized thereto, which first subset may comprise the first nucleic acid molecule and have sequence identity to a first nucleic acid sequence. The second particle may comprise a second subset of the plurality of nucleic acid molecules immobilized thereto, which second subset may have sequence identity to a second nucleic acid sequence. The first subset may be different from the second subset. The first nucleic acid sequence and the second nucleic acid sequence may be identical. Alternatively, the first nucleic acid sequence may be different from the second nucleic acid sequence. The first nucleic acid sequence may comprise a sequence of a sample (e.g., template) nucleic acid molecule, or a complement thereof. The subset of the plurality of nucleic acid molecules may comprise at least about 1,000 nucleic acid molecules (e.g., at least about 500, 1,000, 1,500, 2,000, 2,500, 3,000, 5,000, 10,000, 50,000, 100,000, 200,000, 500,000, 1,000,000, or more nucleic acid molecules).

The plurality of particles may comprise an untreated particle, e.g., a particle that may not comprise a nucleic acid molecule coupled to or capable of coupling to a single-stranded binding moiety. The composition may comprise a plurality of untreated particles. The number of untreated particles in the composition may be greater than the number of treated particles in the composition. Alternatively, the number of treated particles in the composition may be greater than the number of untreated particles in the composition. In some cases, at least about 50%, such as at least about 70% or 90%, of the particles of the composition may be treated particles. Untreated particles and treated particles may be separated from one another using, e.g., one or more processes such as filtration, agitation, centrifugation, magnetic-based sorting, affinity-based sorting, and optical detection-based sorting (e.g., as described herein).

A composition may further comprise one or more reagents, such as one or more reagents for facilitating coupling of a single-stranded binding moiety and a single-stranded portion of a nucleic acid molecule. Such reagents may be included in a reaction mixture, such as a reaction mixture that is separate from the suspension. Examples of reagents useful for facilitating coupling between a single-stranded binding moiety and a single-stranded portion of a nucleic acid molecule include, for example, salts, spermidine, and cobalt hexammine.

In another aspect, the present disclosure provides a method for storing a solution comprising a plurality of particles (e.g., beads). The method may comprise providing a solution (e.g., an aqueous solution) comprising the plurality of particles, where the plurality of particles may comprise a first set of nucleic acid molecules (e.g., DNA or RNA molecules) immobilized thereto. The plurality of particles may comprise any useful number of particles, such as at least 1, 2, 5, 10, 25, 50, 100, 200, 500, 1,000, 2,000, 5,000, 10,000, 50,000, 100,000, 500,000, 1,000,000, 2,000,000, 5,000,000, 10,000,000, or more particles. The first set of nucleic acid molecules may be configured to capture sample nucleic acid molecules (e.g., template nucleic acid molecules) derived from one or more nucleic acid samples (e.g., as described herein). The solution comprising the plurality of particles may contacted with a second set of nucleic acid molecules under conditions sufficient for at least about 50%, such as at least about 60%, 70%, 80%, 90%, 95%, or greater, of first nucleic acid molecules of the first set of nucleic acid molecules to couple to second nucleic acid molecules of the second set of nucleic acid molecules. The second set of nucleic acid molecules may not be the sample nucleic acid molecules. The solution may then be stored for a time period of at least 1 hour, such as at least 2 hours, 3 hours, 4 hours, 6 hours, 8 hours, 12 hours, 18 hours, 24 hours, 48 hours, 3 days, 1 week, 2 weeks, 3 weeks, 4 weeks, 1 month, or longer.

A particle of the plurality of particles may have one or more nucleic acid molecules immobilized thereto. For example, a particle of the plurality of particles may have at least 1, 2, 3, 4, 5, 10, 25, 50, 100, 200, 500, 1,000, 2,000, 5,000, 10,000, 50,000, 100,000, 200,000, 500,000, 1,000,000, or more nucleic acid molecules immobilized thereto. In an example, a particle (e.g., a first particle) of a plurality of particles (e.g., beads) may comprise a first subset of a first set of nucleic acid molecules immobilized thereto, and a second particle of the plurality of particles may comprise a second subset of the first set of nucleic acid molecules immobilized thereto. The second subset may be different from the first subset. The first subset of the first set of nucleic acid molecules may have at least partial sequence identity to a first nucleic acid sequence and the second subset of the first set of nucleic acid molecules have at least partial sequence identity to a second nucleic acid sequence. The first nucleic acid sequence may be different from the second nucleic acid sequence. Alternatively, the first nucleic acid sequence and the second nucleic acid sequence may be identical.

A treated particle prepared according to the methods provided herein may comprise any number of nucleic acid molecules of a first set (e.g., nucleic acid molecules initially immobilized to the particle) hybridized to nucleic acid molecules of a second set (e.g., nucleic acid molecules brought into contact with the particle in a solution, which nucleic acid molecules are not sample nucleic acid molecules). For example, at least about 50% of the first set of nucleic acid molecules may be hybridized to second nucleic acid molecules of the second set of nucleic acid molecules, such as at least about 55%, 60%, 65%, 70%, 75%, 80%, 85%, 90%, 95%, or greater. In an example, at least about 95% of the first set of nucleic acid molecules may be hybridized to second nucleic acid molecules of the second set of nucleic acid molecules. Nucleic acid molecule pairs may be stably hybridized to one another such that nucleic acid molecule pairs are not significantly diminished during storage of a solution comprising a plurality of treated particles. For example, during storage of a solution of a plurality of treated particles, each first nucleic acid molecule of the first set of nucleic acid molecules that is hybridized to a second nucleic acid molecule of the second set of nucleic acid molecules may not hybridize to another nucleic acid molecule of the first set of nucleic acid molecules.

Nucleic acid molecules may be immobilized to a particle via any useful linkage (e.g., as described herein), including, for example, a cleavable linkage (e.g., as described herein). In some cases, nucleic acid molecules may be releasably coupled to a particle such that one or more nucleic acid molecules may be released from a particle (e.g., upon application of an appropriate stimulus, such as a thermal stimulus, chemical stimulus, or light). Nucleic acid molecules immobilized to a particle may be of any useful type and may have any useful features. For example, a nucleic acid molecule immobilized to a particle may comprise DNA or RNA. In an example, the first set of nucleic acid molecules may comprise DNA nucleotides and/or RNA nucleotides. Similarly, the second set of nucleic acid molecules may comprise DNA nucleotides and/or RNA nucleotides. A nucleic acid molecule may comprise any useful nucleotides, including canonical DNA and RNA nucleotides (e.g., adenine, thymine, cytosine, guanine, and uracil-containing nucleotides) and nucleotide analogs (e.g., as described herein). A nucleic acid molecule may comprise any useful number of nucleotides. For example, a nucleic acid molecule immobilized to a particle may comprise at least 6 nucleotides in sequence, such as at least 8, 10, 20, 30, 40, 50, 60, 70, 80, 90, 100, or more nucleotides. A nucleic acid molecule configured to hybridize to a nucleic acid molecule immobilized to a particle (e.g., a nucleic acid molecule of a second set of nucleic acid molecules) may comprise at least 6 nucleotides, such as at least 8, 10, 12, 14, 16, 18, 20, or more nucleotides.

A nucleic acid molecule immobilized to a particle may be a double-stranded nucleic acid molecule. A nucleic acid molecule immobilized to a particle may comprise one or more single-stranded regions. A nucleic acid molecule immobilized to particle may comprise one or more functional sequences (e.g., as described herein), including one or more barcode sequences, unique molecular identifier sequences, sequencing primer sequences, flow cell sequences, adapter sequences, index sequences, or other sequences. Nucleic acid molecules coupled to a given particle may comprise identical nucleic acid sequences. For example, nucleic acid molecules coupled to a given particle may all have sequence identity to one another. In an example, a given particle may comprise a plurality of nucleic acid molecules coupled thereto, where the plurality of nucleic acid molecules includes a first subset of nucleic acid molecules that have sequence identity to one another and a second subset including one or more nucleic acid molecules that do not have sequence identity to nucleic acid molecules of the first subset. In another example, each first nucleic acid molecule of a first set of nucleic acid molecules coupled to a particle may comprise a common nucleic acid sequence. At least a portion of the first nucleic acid molecules of the first set of nucleic acid molecules coupled to the particle may comprise one or more different nucleic acid sequences, such as one or more barcode sequences or unique molecular identifier sequences. For example, the first set of nucleic acid molecules may comprise one or more different nucleic acid sequences. The first set of nucleic acid molecules may comprise a first subset of nucleic acid molecules comprising a first nucleic acid sequence and a second subset of nucleic acid molecules comprising a second nucleic acid sequence, where the first and second nucleic acid sequences are different. The first subset of nucleic acid molecules and the second subset of nucleic acid molecules may both comprise a third nucleic acid sequence, which third nucleic acid sequence may comprise, for example, a poly(thymine) (e.g., poly(T)) or poly(adenine) (e.g., poly(A)) sequence. A second nucleic acid molecule of a second set of nucleic acid molecules may have sequence complementarity to all or a portion of a nucleic acid molecule of a first set of nucleic acid molecules coupled to a particle. For example, a second nucleic acid molecule of the second set of nucleic acid molecules may comprise a sequence that is substantially complementary to a sequence of the first set of nucleic acid molecules. The sequence of the first set of nucleic acid molecules may comprise at least 6 bases, such as at least 6, 8, 10, 12, 14, 16, 18, 20, or more bases.

A solution comprising a plurality of treated particles may be stored for any useful time under any useful conditions. For example, a solution may be stored for at least 1 hour, such as at least about 2 hours, 4 hours, 6 hours, 8 hours, 12 hours, 18 hours, 24 hours, 48 hours, 72 hours, 1 day, 2 days, 3 days, 4 days, 5 days, 6 days, 7 days, 10 days, 2 weeks, 3 weeks, 4 weeks, 1 month, 2 months, 3 months, or longer. A solution may be stored at room temperature. Alternatively, a solution may be stored below room temperature, such as between about 18° C. to about 30° C. A solution may be stored in a freezer. A solution comprising a plurality of treated particles may comprise one or more additional materials including, e.g., one or more stabilizing agents or cryoprotective agents.

A method of storing a solution comprising a plurality of particles may further comprise, subsequent to storing the solution, subjecting the plurality of particles to conditions sufficient to decouple the second nucleic acid molecules of the second set of nucleic acid molecules from the first nucleic acid molecules of the first set of nucleic acid molecules. This may comprise denaturing the nucleic acid molecules via, e.g., application of a chemical or thermal stimulus. For example, a chemical stimulus (e.g., sodium hydroxide) may be used to separate second nucleic acid molecules of a second set of nucleic acid molecules from first nucleic acid molecules immobilized to a plurality of particles. Alternatively, a thermal stimulus may be used to separate second nucleic acid molecules of a second set of nucleic acid molecules from first nucleic acid molecules immobilized to a plurality of particles. In an example, a first nucleic acid molecule of the first set of the nucleic acid molecules immobilized to a plurality of particles hybridized to a second nucleic acid molecule of the second set of nucleic acid molecules may have a melting point between about 35° C. and about 55° C. Raising the temperature of the solution comprising the plurality of particles by inputting thermal energy into the system may result in the separation of the hybridized strands. Other regions of the first nucleic acid molecule, such as other double-stranded regions, if any, may have higher melting points such that thermal energy may be used to selectively denature the second nucleic acid molecule from the first nucleic acid molecule without separating other regions of the first nucleic acid molecule. In another example, second nucleic acid molecules of the second set of nucleic acid molecules hybridized to first nucleic acid molecules immobilized to a plurality of particles may be removed via enzymatic degradation, such as using an exonuclease (e.g., Lambda or T7 exonuclease). After separation of second nucleic acid molecules from first nucleic acid molecules immobilized to the plurality of particles, the first nucleic acid molecules of the first set of nucleic acid molecules immobilized to the plurality of particles may be used in one or more applications such as, for example, hybridization capture of the sample nucleic acid molecules or derivatives thereof, single nucleotide polymorphism (SNP) genotyping of the sample nucleic acid molecules or derivatives thereof, sequencing library capture, synthesis of nucleic acid molecules, on-surface amplification of the sample nucleic acid molecules or derivatives thereof, and downstream processing or analysis of the sample nucleic acid molecules or derivatives thereof.

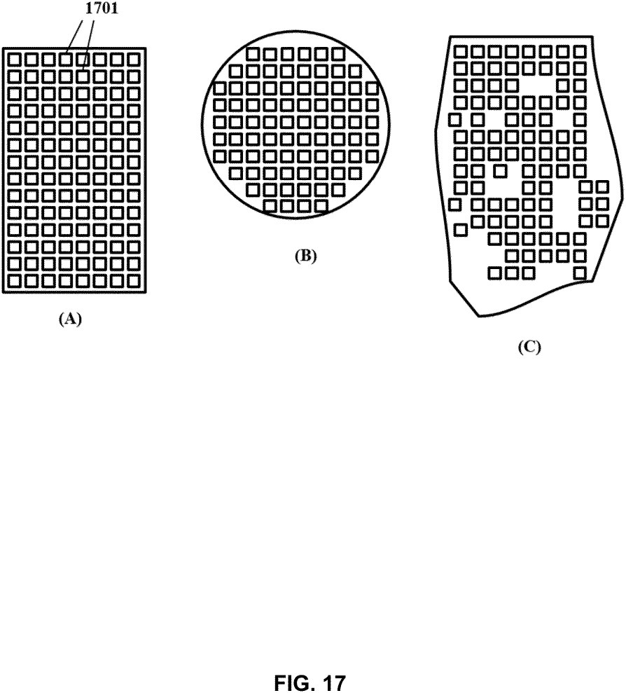

A plurality of particles may be immobilized to a substrate. The plurality of particles may be immobilized to a substrate prior to undergoing treatment as described herein. Alternatively, the plurality of particles may be immobilized to after undergoing treatment as described herein. For example, the plurality of particles may be immobilized to a substrate after removal of second nucleic acid molecules from the plurality of particles (e.g., as described herein). A substrate may comprise a substantially planar array, such that the plurality of particles may be immobilized to a substantially planar array of a substrate. The plurality of particles may be immobilized to a substrate at independently addressable locations, which independently addressable locations may be substantially planar and may comprise one or more wells. The plurality of particles may be immobilized to the substrate in a random pattern. Alternatively, the plurality of particles may be immobilized to the substrate in a predetermined pattern (e.g., as described herein). FIG. 17 illustrates examples of arrays of individually addressable locations 1701 as patterned onto a substrate (e.g., from a top view), with panel A showing a substantially rectangular substrate with regular linear arrays, panel B showing a substantially circular substrate with regular linear arrays, and panel C showing an arbitrarily shaped substrate with irregular arrays. In some cases, the plurality of particles may be immobilized to the substrate with a density of, for example, at least about 1 particle per mm², such as at least about 100, 1,000, 10,000, 100,000, 1,000,000, 10,000,000 particles per mm², or more.

In another aspect, the present disclosure provides a method for nucleic acid processing. The method may comprise providing a solution comprising a plurality of particles. The plurality of particles may comprise any useful number of particles, such as at least 1, 2, 5, 10, 25, 50, 100, 200, 500, 1,000, 2,000, 5,000, 10,000, 50,000, 100,000, 500,000, 1,000,000, 2,000,000, 5,000,000, 10,000,000, or more particles. The plurality of particles may comprise a first set of nucleic acid molecules (e.g., DNA or RNA molecules) immobilized thereto (e.g., as described herein). The first set of nucleic acid molecules may be configured to capture sample nucleic acid molecules (e.g., template nucleic acid molecules) derived from one or more nucleic acid samples (e.g., as described herein). A second set of nucleic acid molecules may also be provided. The second set of nucleic acid molecules may not be the sample nucleic acid molecules. The second set of nucleic acid molecules may comprise sequences that are substantially complementary to sequences of the first set of nucleic acid molecules. The solution may have been stored for a time period of at least 1 hour, such as at least 2 hours, 3 hours, 4 hours, 6 hours, 8 hours, 12 hours, 18 hours, 24 hours, 48 hours, 3 days, 1 week, 2 weeks, 3 weeks, 4 weeks, 1 month, or longer. The solution may have been stored under conditions sufficient for at least about 50%, such as at least about 60%, 70%, 80%, 90%, 95%, or greater, of first nucleic acid molecules of the first set of nucleic acid molecules to couple to second nucleic acid molecules of a second set of nucleic acid molecules. The method may comprise subjecting the plurality of particles to conditions sufficient to decouple the second nucleic acid molecules of the second set of nucleic acid molecules from the first nucleic acid molecules of the first set of nucleic acid molecules.

A particle of the plurality of particles may have one or more nucleic acid molecules immobilized thereto (e.g., as described herein). For example, a particle of the plurality of particles may have at least 1, 2, 3, 4, 5, 10, 25, 50, 100, 200, 500, 1,000, 2,000, 5,000, 10,000, 50,000, 100,000, 1,000,000, 2,000,000, 5,000,000, 10,000,000, or more nucleic acid molecules immobilized thereto. In an example, a particle (e.g., a first particle) of a plurality of particles (e.g., beads) may comprise a first subset of a first set of nucleic acid molecules immobilized thereto, and a second particle of the plurality of particles may comprise a second subset of the first set of nucleic acid molecules immobilized thereto. The second subset may be different from the first subset. The first subset of the first set of nucleic acid molecules may have at least partial sequence identity to a first nucleic acid sequence and the second subset of the first set of nucleic acid molecules have at least partial sequence identity to a second nucleic acid sequence. The first nucleic acid sequence may be different from the second nucleic acid sequence. Alternatively, the first nucleic acid sequence and the second nucleic acid sequence may be identical.

A treated particle prepared according to the methods provided herein may comprise any number of nucleic acid molecules of a first set (e.g., nucleic acid molecules initially immobilized to the particle) hybridized to nucleic acid molecules of a second set (e.g., nucleic acid molecules brought into contact with the particle in a solution, which nucleic acid molecules are not sample nucleic acid molecules). For example, at least about 50% of the first set of nucleic acid molecules may be hybridized to second nucleic acid molecules of the second set of nucleic acid molecules, such as at least about 55%, 60%, 65%, 70%, 75%, 80%, 85%, 90%, 95%, or greater. In an example, at least about 95% of the first set of nucleic acid molecules may be hybridized to second nucleic acid molecules of the second set of nucleic acid molecules. Nucleic acid molecule pairs may be stably hybridized to one another such that nucleic acid molecule pairs are not significantly diminished during storage of a solution comprising a plurality of treated particles. For example, during storage of a solution of a plurality of treated particles, each first nucleic acid molecule of the first set of nucleic acid molecules that is hybridized to a second nucleic acid molecule of the second set of nucleic acid molecules may not hybridize to another nucleic acid molecule of the first set of nucleic acid molecules.

Nucleic acid molecules may be immobilized to a particle via any useful linkage (e.g., as described herein), including, for example, a cleavable linkage (e.g., as described herein). In some cases, nucleic acid molecules may be releasably coupled to a particle such that one or more nucleic acid molecules may be released from a particle (e.g., upon application of an appropriate stimulus, such as a thermal stimulus, chemical stimulus, or light). Nucleic acid molecules immobilized to a particle may be of any useful type and may have any useful features. For example, a nucleic acid molecule immobilized to a particle may comprise DNA or RNA. In an example, the first set of nucleic acid molecules may comprise DNA nucleotides and/or RNA nucleotides. Similarly, the second set of nucleic acid molecules may comprise DNA nucleotides and/or RNA nucleotides. A nucleic acid molecule may comprise any useful nucleotides, including canonical DNA and RNA nucleotides (e.g., adenine, thymine, cytosine, guanine, and uracil-containing nucleotides) and nucleotide analogs (e.g., as described herein). A nucleic acid molecule may comprise any useful number of nucleotides. For example, a nucleic acid molecule immobilized to a particle may comprise at least 6 nucleotides in sequence, such as at least 8, 10, 20, 30, 40, 50, 60, 70, 80, 90, 100, or more nucleotides. A nucleic acid molecule configured to hybridize to a nucleic acid molecule immobilized to a particle (e.g., a nucleic acid molecule of a second set of nucleic acid molecules) may comprise at least 6 nucleotides, such as at least 8, 10, 12, 14, 16, 18, 20, or more nucleotides.

A nucleic acid molecule immobilized to a particle may be a double-stranded nucleic acid molecule. A nucleic acid molecule immobilized to a particle may comprise one or more single-stranded regions. A nucleic acid molecule immobilized to particle may comprise one or more functional sequences (e.g., as described herein), including one or more barcode sequences, unique molecular identifier sequences, sequencing primer sequences, flow cell sequences, adapter sequences, index sequences, or other sequences. Nucleic acid molecules coupled to a given particle may comprise identical nucleic acid sequences. For example, nucleic acid molecules coupled to a given particle may all have sequence identity to one another. In an example, a given particle may comprise a plurality of nucleic acid molecules coupled thereto, where the plurality of nucleic acid molecules includes a first subset of nucleic acid molecules that have sequence identity to one another and a second subset including one or more nucleic acid molecules that do not have sequence identity to nucleic acid molecules of the first subset. In another example, each first nucleic acid molecule of a first set of nucleic acid molecules coupled to a particle may comprise a common nucleic acid sequence. At least a portion of the first nucleic acid molecules of the first set of nucleic acid molecules coupled to the particle may comprise one or more different nucleic acid sequences, such as one or more barcode sequences or unique molecular identifier sequences. For example, the first set of nucleic acid molecules may comprise one or more different nucleic acid sequences. The first set of nucleic acid molecules may comprise a first subset of nucleic acid molecules comprising a first nucleic acid sequence and a second subset of nucleic acid molecules comprising a second nucleic acid sequence, where the first and second nucleic acid sequences are different. The first subset of nucleic acid molecules and the second subset of nucleic acid molecules may both comprise a third nucleic acid sequence, which third nucleic acid sequence may comprise, for example, a poly(thymine) (e.g., poly(T)) or poly(adenine) (e.g., poly(A)) sequence. A second nucleic acid molecule of a second set of nucleic acid molecules may have sequence complementarity to all or a portion of a nucleic acid molecule of a first set of nucleic acid molecules coupled to a particle. For example, a second nucleic acid molecule of the second set of nucleic acid molecules may comprise a sequence that is substantially complementary to a sequence of the first set of nucleic acid molecules. The sequence of the first set of nucleic acid molecules may comprise at least 6 bases, such as at least 6, 8, 10, 12, 14, 16, 18, 20, or more bases.

A solution comprising a plurality of treated particles may be stored for any useful time under any useful conditions. For example, a solution may be stored for at least 1 hour, such as at least about 2 hours, 4 hours, 8 hours, 12 hours, 18 hours, 24 hours, 48 hours, 72 hours, 1 day, 2 days, 3 days, 4 days, 5 days, 6 days, 7 days, 10 days, 2 weeks, 3 weeks, 4 weeks, 1 month, 2 months, 3 months, or longer. A solution may be stored at room temperature. Alternatively, a solution may be stored below room temperature, such as between about 4° C. to about 30° C., such as between about 18° C. to about 30° C. A solution may be stored in a freezer. A solution comprising a plurality of treated particles may comprise one or more additional materials including, e.g., one or more stabilizing agents or cryoprotective agents.

Separating second nucleic acid molecules of a second set of nucleic acid molecules from first nucleic acid molecules immobilized to the plurality of particles (e.g., treated particles) may comprise denaturing the nucleic acid molecules via, e.g., application of a chemical or thermal stimulus. For example, a chemical stimulus (e.g., sodium hydroxide) may be used to separate second nucleic acid molecules of a second set of nucleic acid molecules from first nucleic acid molecules immobilized to a plurality of treated particles. Alternatively, a thermal stimulus may be used to separate second nucleic acid molecules of a second set of nucleic acid molecules from first nucleic acid molecules immobilized to a plurality of treated particles. In an example, a first nucleic acid molecule of the first set of the nucleic acid molecules immobilized to a plurality of particles hybridized to a second nucleic acid molecule of the second set of nucleic acid molecules may have a melting point between about 35° C. and about 55° C. Raising the temperature of the solution comprising the plurality of particles by inputting thermal energy into the system may result in the separation of the hybridized strands. Other regions of the first nucleic acid molecule, such as other double-stranded regions, if any, may have higher melting points such that thermal energy may be used to selectively denature the second nucleic acid molecule from the first nucleic acid molecule without separating other regions of the first nucleic acid molecule. In another example, second nucleic acid molecules of the second set of nucleic acid molecules hybridized to first nucleic acid molecules immobilized to a plurality of particles may be removed via enzymatic degradation (e.g., as described herein). After removing second nucleic acid molecules from the plurality of particles, the first nucleic acid molecules of the first set of nucleic acid molecules immobilized to the plurality of particles may be used in one or more applications such as, for example, hybridization capture of the sample nucleic acid molecules or derivatives thereof, single nucleotide polymorphism (SNP) genotyping of the sample nucleic acid molecules or derivatives thereof, sequencing library capture, synthesis of nucleic acid molecules, on-surface amplification of the sample nucleic acid molecules or derivatives thereof, and downstream processing or analysis of the sample nucleic acid molecules or derivatives thereof.

A plurality of particles may be immobilized to a substrate. The plurality of particles may be immobilized to a substrate prior to undergoing treatment as described herein. Alternatively, the plurality of particles may be immobilized to after undergoing treatment as described herein. For example, the plurality of particles may be immobilized to a substrate after removal of second nucleic acid molecules from the plurality of particles (e.g., as described herein). A substrate may comprise a substantially planar array, such that the plurality of particles may be immobilized to a substantially planar array of a substrate. The plurality of particles may be immobilized to a substrate at independently addressable locations, which independently addressable locations may be substantially planar and may comprise one or more wells. The plurality of particles may be immobilized to the substrate in a random pattern. Alternatively, the plurality of particles may be immobilized to the substrate in a predetermined pattern (e.g., as described herein). In some cases, the plurality of particles may be immobilized to the substrate with a density of, for example, at least about 1 particle per mm², such as at least about 100, 1,000, 10,000, 100,000, 1,000,000, 10,000,000 particles per mm², or more.

The present disclosure also provides kits comprising treated particles and kits for preparing treated particles. A kit may comprise a solution (e.g., aqueous solution) comprising a plurality of particles (e.g., beads). The plurality of particles of the solution may comprise, for example, at least 1, 2, 5, 10, 25, 50, 100, 200, 500, 1,000, 2,000, 5,000, 10,000, 50,000, 100,000, 500,000, 1,000,000, 2,000,000, 5,000,000, 10,000,000, or more particles. The plurality of particles may comprise a first set of nucleic acid molecules (e.g., DNA and/or RNA molecules) comprising first nucleic acid molecules, which nucleic acid molecules may be immobilized to particles of the plurality of particles (e.g., as described herein). The one or more first nucleic acid molecules may be configured to capture sample nucleic acid molecules (e.g., template nucleic acid molecules) derived from one or more nucleic acid samples. A second set of nucleic acid molecules may also be provided. The second set of nucleic acid molecules may be provided in a second solution (e.g., a second aqueous solution). Alternatively, the second set of nucleic acid molecules may be provided in the solution comprising the plurality of particles. The second set of nucleic acid molecules may not be the sample nucleic acid molecules (e.g., template nucleic acid molecules). The second set of nucleic acid molecules may comprise sequences that are substantially complementary to sequences of the first set of nucleic acid molecules such that, upon contacting the plurality of particles with the second set of nucleic acid molecules, at least about 50%, such as at least about 60%, 70%, 80%, 90%, 95%, or greater, of first nucleic acid molecules of the first set of nucleic acid molecules coupled to second nucleic acid molecules of the second set of nucleic acid molecules. Sequences of the first set of nucleic acid molecules may comprise between about 6-20 bases. Sequences of the first set of nucleic acid molecules and the sequences of the second set of nucleic acid molecules may have the same number of nucleotides. Alternatively or additionally, sequences of the first set of nucleic acid molecules and sequences of the second set of nucleic acid molecules may have different numbers of nucleotides. Sequences of the first set of nucleic acid molecules may comprise one or more different nucleic acid sequences. Alternatively, sequences of the first set of nucleic acid molecules may be identical.

A particle of the plurality of particles may have one or more nucleic acid molecules immobilized thereto (e.g., as described herein). For example, a particle of the plurality of particles may have at least 1, 2, 3, 4, 5, 10, 25, 50, 100, 200, 500, 1,000, 2,000, 5,000, 10,000, 50,000, 100,000, 500,000, 1,000,000, 2,000,000, 5,000,000, 10,000,000, or more nucleic acid molecules immobilized thereto. A first particle of the plurality of particles may comprise a first subset of the first set of nucleic acid molecules immobilized thereto, where a second particle of the plurality of particles may comprise a second subset of the first set of nucleic acid molecules immobilized thereto. The second subset may be different from the first subset. The first subset of the first set of nucleic acid molecules may have at least partial sequence identity to a first nucleic acid sequence and the second subset of the first set of nucleic acid molecules have at least partial sequence identity to a second nucleic acid sequence. The first nucleic acid sequence may be different from the second nucleic acid sequence. Alternatively, the first nucleic acid sequence and the second nucleic acid sequence may be identical.

Nucleic acid molecule pairs may be stably hybridized to one another such that nucleic acid molecule pairs are not significantly diminished during storage of a solution comprising a plurality of treated particles. For example, during storage of a solution of a plurality of treated particles, each first nucleic acid molecule of the first set of nucleic acid molecules that is hybridized to a second nucleic acid molecule of the second set of nucleic acid molecules may not hybridize to another nucleic acid molecule of the first set of nucleic acid molecules. A first nucleic acid molecule and a second nucleic acid molecule included in a pair may comprise different number of nucleotides. Alternatively, a first nucleic acid molecule and a second nucleic acid molecule included in a pair may comprise the same number of nucleotides.

Nucleic acid molecules may be immobilized to a particle via any useful linkage (e.g., as described herein), including, for example, a cleavable linkage. In some cases, nucleic acid molecules may be releasably coupled to a particle such that one or more nucleic acid molecules may be released from a particle (e.g., upon application of an appropriate stimulus, such as a thermal stimulus, chemical stimulus, or light). Nucleic acid molecules immobilized to a particle may be of any useful type and may have any useful features. For example, a nucleic acid molecule immobilized to a particle may comprise DNA or RNA. In an example, the first set of nucleic acid molecules may comprise DNA nucleotides and/or RNA nucleotides. Similarly, the second set of nucleic acid molecules may comprise DNA nucleotides and/or RNA nucleotides. A nucleic acid molecule may comprise any useful nucleotides, including canonical DNA and RNA nucleotides (e.g., adenine, thymine, cytosine, guanine, and uracil-containing nucleotides) and nucleotide analogs (e.g., as described herein). A nucleic acid molecule may comprise any useful number of nucleotides. For example, a nucleic acid molecule immobilized to a particle may comprise at least 6 nucleotides in sequence, such as at least 8, 10, 20, 30, 40, 50, 60, 70, 80, 90, 100, or more nucleotides. A nucleic acid molecule configured to hybridize to a nucleic acid molecule immobilized to a particle (e.g., a nucleic acid molecule of a second set of nucleic acid molecules) may comprise at least 6 nucleotides, such as at least 8, 10, 12, 14, 16, 18, 20, or more nucleotides.

A nucleic acid molecule immobilized to a particle may be a double-stranded nucleic acid molecule. A nucleic acid molecule immobilized to a particle may comprise one or more single-stranded regions. A nucleic acid molecule immobilized to particle may comprise one or more functional sequences (e.g., as described herein), including one or more barcode sequences, unique molecular identifier sequences, sequencing primer sequences, flow cell sequences, adapter sequences, index sequences, or other sequences. Nucleic acid molecules coupled to a given particle may comprise identical nucleic acid sequences. For example, nucleic acid molecules coupled to a given particle may all have sequence identity to one another. In an example, a given particle may comprise a plurality of nucleic acid molecules coupled thereto, where the plurality of nucleic acid molecules includes a first subset of nucleic acid molecules that have sequence identity to one another and a second subset including one or more nucleic acid molecules that do not have sequence identity to nucleic acid molecules of the first subset. In another example, each first nucleic acid molecule of a first set of nucleic acid molecules coupled to a particle may comprise a common nucleic acid sequence. At least a portion of the first nucleic acid molecules of the first set of nucleic acid molecules coupled to the particle may comprise one or more different nucleic acid sequences, such as one or more barcode sequences or unique molecular identifier sequences. For example, the first set of nucleic acid molecules may comprise one or more different nucleic acid sequences. The first set of nucleic acid molecules may comprise a first subset of nucleic acid molecules comprising a first nucleic acid sequence and a second subset of nucleic acid molecules comprising a second nucleic acid sequence, where the first and second nucleic acid sequences are different. The first subset of nucleic acid molecules and the second subset of nucleic acid molecules may both comprise a third nucleic acid sequence, which third nucleic acid sequence may comprise, for example, a poly(thymine) (e.g., poly(T)) or poly(adenine) (e.g., poly(A)) sequence. A second nucleic acid molecule of a second set of nucleic acid molecules may have sequence complementarity to all or a portion of a nucleic acid molecule of a first set of nucleic acid molecules coupled to a particle. For example, a second nucleic acid molecule of the second set of nucleic acid molecules may comprise a sequence that is substantially complementary to a sequence of the first set of nucleic acid molecules. The sequence of the first set of nucleic acid molecules may comprise at least 6 bases, such as at least 6, 8, 10, 12, 14, 16, 18, 20, or more bases. A second nucleic acid molecule of the second set of nucleic acid molecules may comprise a sequence that is substantially complementary to a sequence of the first set of nucleic acid molecules. Each first nucleic acid molecule of the first set of nucleic acid molecules may comprise a common nucleic acid sequence. The first set of nucleic acid molecules may comprise one or more different nucleic acid sequences. The first set of nucleic acid molecules may comprise a plurality of priming sequences. The plurality of priming sequences may comprise a plurality of poly(T) sequences. Alternatively or additionally, the plurality of priming sequences may comprise a plurality of random N-mer sequences.

The materials of a kit may be stored for any useful time under any useful conditions. For example, a solution may be stored for at least 1 hour, such as at least about 2 hours, 4 hours, 6 hours, 8 hours, 12 hours, 18 hours, 24 hours, 48 hours, 72 hours, 1 day, 2 days, 3 days, 4 days, 5 days, 6 days, 7 days, 10 days, 2 weeks, 3 weeks, 4 weeks, 1 month, 2 months, 3 months, or longer. A solution may be stored at room temperature. Alternatively, a solution may be stored below room temperature, such as between about 4° C. to about 30° C., such as between about 18° C. to about 30° C. A solution may be stored at a freezing temperature (e.g., in a freezer). A solution may comprise one or more agents to facilitate storage of particles included therein, including, for example, a stabilizing agent or cryoprotective agent. Where a kit comprises multiple solutions, such as a first solution comprising a plurality of particles and a second solution comprising a second set of nucleic acid molecules, the solutions may be stored together. Alternatively, the solutions of the kit may be stored separately.

A kit may include an agent configured to separate second nucleic acid molecules from first nucleic acid molecules immobilized to a plurality of particles. Such an agent may comprise a chemical stimulus such as sodium hydroxide. Alternatively, pairs of coupled nucleic acid molecules may be configured to be separated (e.g., denatured) using a thermal stimulus. In an example, a first nucleic acid molecule of the first set of the nucleic acid molecules immobilized to a plurality of particles hybridized to a second nucleic acid molecule of the second set of nucleic acid molecules may have a melting point between about 35° C. and about 55° C., such that raising the temperature of a solution comprising the plurality of treated particles by inputting thermal energy into the system may result in the separation of hybridized strands. Other regions of the first nucleic acid molecule, such as other double-stranded regions, if any, may have higher melting points such that thermal energy may be used to selectively denature the second nucleic acid molecule from the first nucleic acid molecule without separating other regions of the first nucleic acid molecule. In another example, second nucleic acid molecules of the second set of nucleic acid molecules hybridized to first nucleic acid molecules immobilized to a plurality of particles may be configured to be removed via enzymatic degradation.

The plurality of particles of a solution of a kit may be immobilized to a substrate. A substrate may comprise a substantially planar array, such that the plurality of particles may be immobilized to a substantially planar array of a substrate. The plurality of particles may be immobilized to a substrate at independently addressable locations, which independently addressable locations may be substantially planar. In some cases, the independent addressable locations may comprise one or more wells, one or more protrusions, and/or one or more depressions. The plurality of particles may be immobilized to the substrate in a random pattern. Alternatively, the plurality of particles may be immobilized to the substrate in a predetermined pattern (e.g., as described herein). In some cases, the plurality of particles may be immobilized to the substrate with a density of, for example, at least about 1 particle per mm², such as at least about 100, 1,000, 10,000, 100,000, 1,000,000, 10,000,000 particles per mm², or more.

Subsequent to generating or receiving a plurality of particles (e.g., subsequent to attaching a nucleic acid template to a support, subsequent to an amplification reaction on or with the support, etc.), wherein at least some of the plurality of particles comprises a nucleic acid molecule immobilized thereto, the plurality of particles may have a first maximum dimension (average). A particle may be a support, such as a bead. The maximum dimension may be a diameter, width, length, depth, or any other dimension which has the greatest value from those of different dimensions. In order to efficiently immobilize, or enable fitting of, the particles onto individually addressable locations on a substrate having a predetermined pitch (e.g., 1.5 micrometer pitch, 1.8 micrometer pitch, 2.0 micrometer pitch, any other pitch, etc.), it may be beneficial to decrease the first maximum dimension (average) of the plurality of particles to a second maximum dimension before or upon loading, so as to more densely pack the particles onto a substrate having a set surface area. Further, in order to efficiently immobilize, or enable fitting of, the particles onto individually addressable locations on a substrate having a predetermined pitch, it may be beneficial to minimize or mitigate particle-to-particle repulsion effects resulting from the respective overall negative charge of the nucleic acids immobilized to the particles, so as to more densely pack the particles onto a substrate having a set surface area. Incubation of the plurality of particles with a cation, such as a divalent cation (e.g., Ca²⁺ or Mg²⁺), prior to loading of said plurality of particles may address at least the abovementioned technical problems to be solved. The cation may facilitate shrinking of particle sizes, as well as screening of respective negative charges between adjacent or neighboring particles. However, the present disclosure has recognized that the incubation of particles with a cation for a prolonged period of time may also cause particle aggregation which can disrupt sequencing quality down the line (e.g., upon detection of optical signals during sequencing). For example, where incubation with magnesium ion is prolonged, it may be possible that the magnesium ion may begin acting as a crosslinker between two phosphate anions from the nucleic acids (e.g., DNA) on neighboring particles, to cause particle aggregation between such neighboring particles. Thus, recognized herein are methods for optimizing the cation-particle interaction to reduce particle aggregation upon loading of a plurality of particles onto a substrate surface. Further, the present disclosure discovers the unexpected technical effects of particle aggregation reduction with use of certain divalent cations, such as Mg²⁺ and Ca²⁺, over other divalent cations, such as Zn²⁺, which was discovered to aggravate and cause catastrophic aggregation instead of the desired reduction of same.

In some instances, reducing particle aggregation may comprise a method for dispensing a plurality of particles. A particle may be a support, such as a bead. In some instances, a method for dispensing a plurality of particles onto a substrate may comprise: (a) incubating said plurality of particles with a first buffer solution, wherein said first buffer solution is substantially depleted of cations, wherein each of at least some of said plurality of particles comprises a nucleic acid molecule immobilized thereto; (b) loading said substrate with a second buffer solution, wherein said second buffer solution comprises said cations; and (c) dispensing said plurality of particles onto said substrate.

In some instances, a method for dispensing a plurality of particles onto a substrate may comprise: (a) incubating said plurality of particles with a first buffer solution, wherein said first buffer solution is substantially depleted of cations, wherein each of at least some of said plurality of particles comprises a nucleic acid molecule immobilized thereto; (b) loading said substrate with a second buffer solution, wherein said second buffer solution comprises said cations; (c) incubating said substrate with said second buffer solution; and (d) dispensing said plurality of particles onto said substrate.

In some instances, a method for dispensing a plurality of particles onto a substrate may comprise: (a) incubating said plurality of particles with a first buffer solution, wherein said first buffer solution is substantially depleted of cations, wherein each of at least some of said plurality of particles comprises a nucleic acid molecule immobilized thereto; (b) loading said substrate with a second buffer solution, wherein said second buffer solution comprises said cations; (c) forming a layer of said cations on said substrate; and (d) dispensing said plurality of particles onto said substrate.

In some instances, a method for dispensing a plurality of particles onto a substrate may comprise: (a) incubating said plurality of particles with a first buffer solution, wherein said first buffer solution is substantially depleted of cations, wherein each of at least some of said plurality of particles comprises a nucleic acid molecule immobilized thereto; (b) loading said substrate with a second buffer solution, wherein said second buffer solution comprises said cations; (c) dispensing said plurality of particles onto said substrate; and (d) immobilizing said plurality of particles to said substrate.

In some instances, a method for dispensing a plurality of particles onto a substrate may comprise: (a) incubating said plurality of particles with a first buffer solution, wherein said first buffer solution is substantially depleted of cations, wherein each of at least some of said plurality of particles comprises a nucleic acid molecule immobilized thereto; (b) loading said substrate with a second buffer solution, wherein said second buffer solution comprises said cations; (c) incubating said substrate with said second buffer solution; (d) forming a layer of said cations on said substrate; (e) dispensing said plurality of particles onto said substrate; and (f) immobilizing said plurality of particles to said substrate.

In some instances, a method for dispensing a plurality of particles onto a substrate may comprise the steps of: (a) incubating said plurality of particles with a first buffer solution, wherein said first buffer solution is substantially depleted of cations, wherein each of at least some of said plurality of particles comprises a nucleic acid molecule immobilized thereto; (b) loading said substrate with a second buffer solution, wherein said second buffer solution comprises said cations; (c) dispensing said plurality of particles onto said substrate.

In some instances, the method may comprise incubating said substrate with said second buffer solution prior to, during, or subsequent to incubating said plurality of particles with a first buffer solution. In some cases, the method may comprise incubating said substrate with said second buffer solution subsequent to loading said substrate with a second buffer solution. In some cases, the method may comprise incubating said substrate with said second buffer solution prior to forming a layer of said cations on said substrate. In some cases, the method may comprise incubating said substrate with said second buffer solution prior to dispensing said plurality of particles onto said substrate. In some cases, the method may comprise incubating said substrate with said second buffer solution prior to immobilizing said plurality of particles to said substrate.

In some instances, the method may comprise forming a layer of said cations on said substrate prior to, during, or subsequent to incubating said plurality of particles with a first buffer solution. In some cases, the method may comprise forming a layer of said cations on said substrate subsequent to loading said substrate with a second buffer solution. In some cases, the method may comprise forming a layer of said cations on said substrate subsequent to incubating said substrate with said second buffer solution. In some cases, the method may comprise forming a layer of said cations on said substrate prior to dispensing said plurality of particles onto said substrate. In some cases, the method may comprise forming a layer of said cations on said substrate prior to immobilizing said plurality of particles to said substrate.

In some instances, the method may comprise immobilizing said plurality of particles to said substrate during or subsequent to incubating said plurality of particles with a first buffer solution. In some cases, the method may comprise immobilizing said plurality of particles to said substrate subsequent to loading said substrate with a second buffer solution. In some cases, the method may comprise immobilizing said plurality of particles to said substrate subsequent to incubating said substrate with said second buffer solution. In some cases, the method may comprise immobilizing said plurality of particles to said substrate subsequent to forming a layer of said cations on said substrate. In some cases, the method may comprise immobilizing said plurality of particles to said substrate subsequent to dispensing said plurality of particles onto said substrate.

In some instances, incubating a plurality of particles with a first buffer solution may comprise a first incubation time at least about 0.1 minute, 0.2 minute, 0.3 minutes, 0.4 minutes, 0.5 minutes, 0.6 minutes, 0.7 minutes, 0.8 minutes, 0.9 minutes, 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 60 minutes, 2 hours, 3 hours, 4 hours or more. Alternatively or in addition, incubating a plurality of particles with a first buffer solution may comprise a first incubation time of at most about 4 hours, 3 hours, 2 hours, 60 minutes, 50 minutes, 40 minutes, 30 minutes, 20 minutes, 10 minutes, 9 minutes, 8 minutes, 7 minutes, 6 minutes, 5 minutes, 3 minutes, 2 minutes, 1 minutes, 0.9 minutes, 0.8 minutes, 0.7 minutes, 0.6 minutes, 0.5 minutes, 0.4 minutes, 0.3 minutes, 0.2 minute, 0.1 minute or less.

In some instances, incubating a plurality of particles with a second buffer solution may comprise a second incubation time of at least about 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 60 minutes, 2 hours, 3 hours, 4 hours or more. Alternatively or in addition, incubating a plurality of particles with a second buffer solution may comprise a second incubation time of at most about 4 hours, 3 hours, 2 hours, 60 minutes, 50 minutes, 40 minutes, 30 minutes, 20 minutes, 10 minutes, 9 minutes, 8 minutes, 7 minutes, 6 minutes, 5 minutes, 3 minutes, 2 minutes, 1 minutes, or less.

In some instances, the cations may comprise divalent cations. Alternatively or in addition, the cations may comprise monovalent cations, divalent cations, trivalent cations, quadrivalent cations, or pentavalent cations. In some cases, the cations may comprise magnesium ions and/or calcium ions. In some cases, the cations may comprise Mg²⁺ or Ca²⁺. In some instances, the cations may be selected from the group consisting of magnesium ions and calcium ions. In some cases, the cations may be selected from the group consisting of Mg²⁺ and Ca²⁺. In some cases, the cations may comprise magnesium ions. In some cases, the cations may comprise Mg²⁺. In some cases, the cations may comprise calcium ions. In other cases, the cations may comprise Ca²⁺. In other cases, the cations may comprise electrically charged spermine ions. In some cases, spermine ions may comprise spermine¹⁺, spermine²⁺, spermine³⁺, spermine⁴⁺, or a combination thereof. In some cases, the cations may comprise ions of aluminum, barium, bismuth, cadmium, calcium, cesium, chromium, cobalt, copper, copper, hydrogen, iron, iron, lead, lithium, magnesium, mercury, mercury, nickel, potassium, rubidium, silver, sodium, strontium, tin, or spermine. In some cases, the cations may comprise Al³⁺, Ba²⁺, Bi³⁺, Cd²⁺, Ca¹⁺, Ca²⁺, Cs¹⁺, Cr^{H,} Co²⁺, Cu¹⁺, Cu²⁺, H¹⁺, Fe²⁺, Fe³⁺, Pb²⁺, Li¹⁺, Mg¹⁺, Mg²⁺, Hg2²⁺, Hg²⁺, Ni²⁺, K¹⁺, Rb¹⁺, Ag¹⁺, Na¹⁺, Sr²⁺, Sn²⁺, spermine¹⁺, spermine²⁺, spermine³⁺, or spermine⁴⁺.

In some cases, the cations may comprise magnesium ions, calcium ions, and/or spermine ions. In some cases, the cations may comprise spermine ions. In some cases, the cations may comprise a nitrogen atom. In other cases, the cations may comprise at least 1 nitrogen atom. In some cases, the cations may comprise more than 1 nitrogen atom. In some cases, the cations may comprise an amine group. In other cases, the cations may comprise at least 1 amine group. In some cases, the cations may comprise more than 1 amine group. In some cases, the cations may comprise a tetramine. In some cases, the cations may comprise an alkane. In some cases, the cations may comprise a polyalkane. In some cases, the cations may comprise a tetradecane. In some cases, the cations may comprise a polyazaalkane. In some cases, the cations may have nitrogen atoms replacing carbon atoms at positions 1, 5, 10, or 14 of a polyazaalkane. In some cases, the cations may have nitrogen atoms replacing carbon atoms at positions 1, 5, 10, and 14 of a polyazaalkane. In some cases, the cations may have a nitrogen replacing a carbon at position 1 of a polyazaalkane. In some cases, the cations may have a nitrogen replacing a carbon at position 5 of a polyazaalkane. In some cases, the cations may have a nitrogen replacing a carbon at position 10 of a polyazaalkane. In some cases, the cations may have a nitrogen replacing a carbon at position 14 of a polyazaalkane. In some cases, the cations may have a nitrogen replacing a carbon at positions 1, 2, 3, 4, 5, 6, 7, 8, 9, 10, 11, 12, 13, or 14 of a polyazaalkane.

In some cases, a first buffer solution may be depleted or substantially depleted of cations. In some cases, a first buffer solution may be depleted or substantially depleted of monovalent cations, divalent cations, trivalent cations, quadrivalent cations, or pentavalent cations. In some cases, a first buffer solution may be depleted or substantially depleted of calcium ions, magnesium ions, or spermine ions. In some cases, a first buffer solution may be depleted or substantially depleted of Ca²⁺, Mg²⁺, or spermine⁴⁺, or a combination thereof. In some cases, a solution depleted or substantially depleted of ions may comprise at most 1 nanomolar (nM), 1 picomolar (pM), 1 femtomolar (fM), or 1 attomolar (aM) cations. In some cases, a solution depleted or substantially depleted of monovalent cations, divalent cations, trivalent cations, quadrivalent cations, or pentavalent cations may comprise at most 1 nM, 1 pM, 1 fM, or 1 aM of monovalent cations, divalent cations, trivalent cations, quadrivalent cations, or pentavalent cations. In some cases, a solution depleted or substantially depleted of calcium ions, magnesium ions, or spermine ions may comprise at most 1 nM, 1 pM, 1 fM, or 1 aM of calcium ions, magnesium ions, or spermine ions. In some cases, a solution depleted or substantially depleted of Ca²⁺, Mg²⁺, or spermine⁴⁺ may comprise at most 1 nM, 1 pM, 1 fM, or 1 aM of Ca²⁺, Mg²⁺, or spermine⁴⁺.

In some instances, a first buffer solution may be free of cations. In some instances, a first buffer solution may be free of divalent cations. In some cases, a first buffer may be free of calcium ions, magnesium ions, spermine ions, or a combination thereof. In some cases, a first buffer may be free of Ca²⁺, Mg²⁺, spermine⁴⁺, or a combination thereof.

In some cases, a second buffer solution may comprise at least about 5 mM, 10 mM, 15 mM, 20 mM, 25 mM, 30 mM, 35 mM, 40 mM, about 45 mM, 50 mM, 55 mM, 60 mM, 65 mM, 70 mM, 75 mM, 80 mM, 85 mM, 90 mM, 95 mM, or 100 mM of cations. In some instances, a second buffer solution may comprise at least about 1 nM, 10 nM, 100 nM, 1 µM, 10 µM, 100 µM, 1 mM, 10 mM, 100 mM, 1 M, or 10 M of cations. In some cases, a second buffer solution may comprise at most about 1 nM 10 nM, 100 nM, 1 µM, 10 µM, 100 µM, 1 mM, 10 mM, 100 mM, 1 M, or 10 M of cations. In some cases, the cations are monovalent cations, divalent cations, trivalent cations, quadrivalent cations, or pentavalent cations. In some cases, the cations are calcium ions. In some cases, the cations are magnesium ions. In some cases, the cations are spermine ions. In some cases, the cations are Ca²⁺. In some cases, the cations are Mg²⁺. In some cases, the cations are spermine⁴⁺. In some cases, the cations are a combination of monovalent, divalent, trivalent, quadrivalent, or pentavalent etc. cations.

In some instances, a second buffer solution may comprise a single-stranded nucleic binding (SSB) protein and a cation. In some cases, the cation may comprise a calcium ion, a magnesium ion, or a spermine ion. In some cases, the cation may comprise Ca²⁺, Mg²⁺, or spermine⁴⁺. In some cases, the SSB protein may comprise a T4 phage-derived SSB protein, an Escherichia coli-derived SSB protein, an Extreme Thermostable SSB protein, or a combination thereof. In some cases, a second buffer solution may comprise at least about 5 mM, 10 mM, 15 mM, 20 mM, 25 mM, 30 mM, 35 mM, 40 mM, about 45 mM, 50 mM, 55 mM, 60 mM, 65 mM, 70 mM, 75 mM, 80 mM, 85 mM, 90 mM, 95 mM, or 100 mM or more of cations; and at least about 1 nM, 10 nM, 100 nM, 1 µM, 10 µM, 100 µM, 1 mM, 10 mM, 100 mM, 1 M, or 10 M of cations; and at least about 1.0 nM, 2.0 nM, 5.0 nM, 10.0 nM, 20.0 nM, 50.0 nM, 100.0 nM, 0.2 µM,0.3 µM,0.4 µM,0.5 µM,0.75 µM,1.0 µM,1.1 µM,1.25 µM,1.5 µM,1.75 µM,2.0 µM,2.5 µM,3.0 µM,4.0 µM,5.0 µM,6.0 µM,7.0 µM,8.0 µM,9.0 µM,10.0 µM,12.0 µM, 14.0 µM,16.0 µM,18.0 µM,20.0 µM,50.0 µM,100.0 µM,200.0 µM, 250.0 µM,300.0 µM, 350.0 µM, 400.0 µM, 450.0 µM, 500.0 µM, 600.0 µM, 700.0 µM, 800.0 µM, 900.0 µM, 1 mM or more SSB proteins. In some cases, a second buffer solution may comprise at most about 5 mM, 10 mM, 15 mM, 20 mM, 25 mM, 30 mM, 35 mM, 40 mM, about 45 mM, 50 mM, 55 mM, 60 mM, 65 mM, 70 mM, 75 mM, 80 mM, 85 mM, 90 mM, 95 mM, or 100 mM of cations. In some instances, a second buffer solution may comprise at most about 1 nM, 10 nM, 100 nM, 1 µM, 10 µM, 100 µM, 1 mM, 10 mM, 100 mM, 1 M, or 10 M of cations; and at least about 1.0 nM, 2.0 nM, 5.0 nM, 10.0 nM, 20.0 nM, 50.0 nM, 100.0 nM, 0.2 µM, 0.3 µM, 0.4 µM, 0.5 µM,0.75 µM,1.0 µM,1.1 µM,1.25 µM,1.5 µM,1.75 µM,2.0 µM,2.5 µM,3.0 µM,4.0 µM,5.0 µM,6.0 µM,7.0 µM,8.0 µM,9.0 µM,10.0 µM,12.0 µM,14.0 µM,16.0 µM,18.0 µM, 20.0 µM,50.0 µM,100.0 µM,200.0 µM, 250.0 µM,300.0 µM, 350.0 µM,400.0 µM, 450.0 µM, 500.0 µM,600.0 µM,700.0 µM,800.0 µM,900.0 µM,1 mM of SSB proteins.

In some instances, a layer of cations formed on the substrate may have a thickness of from about 1 nanometer (nm) to about 10 nm, from about 1 nm to about 100 nm, from about 1 nm to about 1 micrometer (µm), from about 1 nm to about 10 µm, from about 1 nm to about 100 µm, or from about 1 nm to about 1 millimeter (mm). In some cases, a layer of cations formed on the substrate may have a thickness about 1 µm to about 40 µm, from about 1 µm to about 39 µm, from about 2 µm to about 38 µm, from about 3 µm to about 37 µm, from about 4 µm to about 36 µm, from about 5 µm to about 35 µm, from about 6 µm to about 34 µm, from about 7 µm to about 33 µm, from about 8 µm to about 32 µm, from about 9 µm to about 31 µm, from about 10 µm to about 30 µm, from about 11 µm to about 29 µm, from about 12 µm to about 28 µm, from about 13 µm to about 27 µm, from about 14 µm to about 26 µm, from about 15 µm to about 25 µm, from about 16 µm to about 24 µm, from about 17 µm to about 23 µm, from about 18 µm to about 22 µm, from about 19 µm to about 21 µm, from about 1 µm to about 20 µm, from about 5 µm to about 20 µm, from about 10 µm to about 20 µm, from about 15 µm to about 20 µm, from about 10 µm to about 25 µm, from about 10 µm to about 30 µm, from about 10 µm to about 35 µm, from about 10 µm to about 40 µm, from about 10 µm to about 20 µm, from about 10 µm to about 25 µm, from about 10 µm to about 30 µm, from about 10 µm to about 35 µm, from about 10 µm to about 40 µm, from about 5 µm to about 20 µm, from about 4 µm to about 20 µm, from about 3 µm to about 20 µm, from about 2 µm to about 20 µm, or from about 1 µm to about 20 µm. In some instances, a layer of cations formed on the substrate may have a thickness of at least about 0.1 nm, 0.2 nm, 0.5 nm, 1 nm, 2 nm, 5 nm, 10 nm, 20 nm, 50 nm, 100 nm, 200 nm, 500 nm, 1 µm, 2 µm, 5 µm, 10 µm, 20 µm, 50 µm, 100 µm, 200 µm, 500 µm, 1 mm, or more than at least about 1 mm.

In some instances, a first buffer solution comprises a Tris buffer solution. In some instances, a second buffer solution comprises a Tris buffer solution. In some cases, a Tris buffer solution comprises about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM. In some cases, a Tris buffer solution comprises at least about 1 nM, 10 nM, 100 nM, 1 µM, 10 µM, 100 µM, 1 mM, 10 mM, or 100 mM of Tris. In some cases, a Tris buffer solution comprises at most about 1 nM, 10 nM, 100 nM, 1 µM, 10 µM, 100 µM, 1 mM, 10 mM, or 100 mM of Tris.

In some instances, a first buffer solution may have a pH of about 3, 3.5, 4, 4.5, 5, 5.5, 6, 6.5, 7, 7.5, 8, 8.5, 9, 9.5, or 10. In some cases, a first buffer solution may have a pH of at least about 1, 2, 3, 4, 5, 6, 7, 8, 9, 10, 11, 12, 13, or 14. In some cases, a first buffer solution may have a pH of at most about 1, 2, 3, 4, 5, 6, 7, 8, 9, 10, 11, 12, 13, or 14. In some instances, a second buffer solution may have a pH of about 3, 3.5, 4, 4.5, 5, 5.5, 6, 6.5, 7, 7.5, 8, 8.5, 9, 9.5, or 10. In some cases, a second buffer solution may have a pH of at least about 1, 2, 3, 4, 5, 6, 7, 8, 9, 10, 11, 12, 13, or 14. In some cases, a second buffer solution may have a pH of at most about 1, 2, 3, 4, 5, 6, 7, 8, 9, 10, 11, 12, 13, or 14.

In some instances, a second buffer solution may comprise about 0.01%, 0.015%, 0.02, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.11%, 0.12%, 0.13%, 0.14%, 0.15%, 0.16%, 0.17%, 0.18%, 0.19%, or 0.2% Tergitol by volume. In some cases, a second buffer solution may comprise at least about 0.000001%, 0.00001%, 0.0001%, 0.001%, 0.01%, 0.1%, 1%, or 10% Tergitol by volume. In some cases, a second buffer solution may comprise at most about 0.000001%, 0.00001%, 0.0001%, 0.001%, 0.01%, 0.1%, 1%, or 10% Tergitol by volume.

In some instances, a first buffer solution may have a volume of less than about 1 mL, 0.9, 0.8 mL, 0.7 mL, 0.6 mL, 0.5 mL, 0.4 mL, 0.3 mL, 0.2 mL, 0.1 mL, 90 µL, 80 µL, 70 µL, 60 µL, 50 µL, 45 µL, 40 µL, 35 µL, 30 µL, 25 µL, 20 µL, 15 µL, 10 µL, or 5 µL.

In some instances, the plurality of particles may comprise at least about 10, 100, 1000, 10,000, 100,000, 1,000,000, 10,000,000, 100,000,000, 1,000,000,000, 10,000,000,000, 100,000,000,000 or more particles. In some cases, the plurality of particles may comprise at least about 100,000, 10,000,000, or 1,000,000,000 particles. In some cases, the plurality of particles may comprise at most about 100,000,000,000, 10,000,000,000, 1,000,000,000, 100,000,000, 10,000,000, 1,000,000, 100,000, 10,000, 1000, 100, or 10 particles. In some cases, the plurality of particles may comprise at most about 100,000,000,000, 10,000,000,000, or 1,000,000,000 particles. In some cases, the plurality of particles may comprise about 100,000,000,000, 10,000,000,000, 1,000,000,000, 100,000,000, 10,000,000, 1,000,000, 100,000, 10,000, 1000, 100, or 10 particles. In some cases, the plurality of particles may comprise about 100,000, 10,000,000, or 1,000,000,000 particles.

In some instances, the plurality of particles may comprise a concentration of at least 100,000, 200,000, 500,000, 1,000,000, 2,000,000, 5,000,000, 10,000,000, or 20,000,000 particles per µL of said first buffer. In some cases, the first buffer solution comprising the plurality of particles comprises a concentration of at least about 50,000,000 particles per µL or at least about 100,000,000 particles per µL of said first buffer. In some instances, the plurality of particles may comprise a concentration of at most 100,000, 200,000, 500,000, 1,000,000, 2,000,000, or 5,000,000 particles per µL of said first buffer. In some cases, the first buffer solution comprising the plurality of particles comprises a concentration of at most about 10,000,000, 20,000,000, 50,000,000, or 100,000,000 particles per µL of said first buffer.

In some instances, subsequent to loading, at least about 80%, 81%, 82%, 83%, 84%, 85%, 86%, 87%, 88%, 89%, 90%, 91%, 92%, 93%, 94%, 95%, 96%, 97%, 98%, 99%, or 100% of the independently addressable locations has at least one of said plurality of particles immobilized thereto. In some cases, at least about 50%, 55%, 60%, 65%, or 70% of the independently addressable locations has at least one of said plurality of particles immobilized thereto.

In some instances, the center of each of said plurality of individually addressable locations is separated by about 1 µm, 1.1 µm, 1.15 µm, 1.2 µm, 1.25 µm, 1.3 µm, 1.4 µm, 1.5 µm, 1.6 µm, 1.7 µm, 1.75 µm, 1.8 µm, 1.9 µm, 2 µm, 2.25 µm, 2.5 µm, 2.75 µm, or 3 µm. In some cases, the center of each of said plurality of individually addressable locations is separated by fewer than about 100 µm, 90 µm, 80 µm, 70 µm, 60 µm, 50 µm, 40 µm, 30 µm, 20 µm, or 10 µm.

In some instances, a particle may shrink upon contacting a second buffer solution. In some cases, a particle may shrink by about 5%, 10%, 15%, 20%, 25%, 30%, 35%, 40%, 45%, 50%, 55%, 60%, 65%, 70%, 75%, 80%, 85%, 90%, 95%, or 99%.

### Nucleic Acid Sequencing

Without wishing to be bound by theory, the methods, systems, and compositions provided herein may facilitate improved loading of particles with template nucleic acid molecules by, e.g., compaction of charge density. The methods, systems, and compositions provided herein may also allow for decreased particle aggregation by reducing hybridization of like sequences on separate particles (see, e.g., FIGS. 8A and 8B). The methods, systems, and compositions provided herein may provide for improved sequence quality, such as by reducing long range context issues, which may inhibit polymerase activity. Additional details of systems useful for processing nucleic acid molecules can be found in, for example, U.S. Pat. Application No. 17/155,226 and U.S. Pat. No. US10,900,078, each of which are herein incorporated by reference in their entireties.

Sequencing (e.g., sequencing by synthesis) methods may comprise the use of particles (e.g., beads) coated with single-stranded nucleic acid molecules (e.g., ssDNA), such as single-stranded nucleic acid molecules primed with a PA-26 primer. Depending on the sequence, these single-stranded nucleic acid molecules may have significant secondary structure, which may be challenging to sequence through. Moreover, a single-stranded nucleic acid molecule coupled to a first particle may efficiently hybridize to single-stranded nucleic acid molecule coupled to a second particle if the sequences are compatible. Using human genomic (HG) DNA compounds this issue, as the human genome is highly repetitive, making it very likely that a given particle may encounter a compatible particle in a solution comprising a plurality of particles. If a particle does encounter a compatible particle, this interaction may be very stable and difficult to break up. Most importantly, such particle aggregates are impossible to sequence. Keeping particles separate is therefore paramount for high-throughput sequencing. As single-stranded nucleic acid molecules are a primary driver of such aggregation, methods, systems, and compositions for reducing the single-stranded nucleic acid molecule content of particles have the potential to improve sequencing. Single-stranded binding moieties such as single-strand binding proteins (SSBs) are known to bind tightly to single-stranded nucleic acid molecules (e.g., ssDNA) and may actually stimulate DNA polymerase activity. Accordingly, single-stranded binding moieties such as SSBs may have significant impacts on bead aggregation and sequencing quality.

Processing of a template nucleic acid molecule may be performed using a substrate comprising an array having immobilized thereto the template nucleic acid molecule. The template nucleic acid molecule may be a sample nucleic acid molecule derived from a nucleic acid sample (e.g., as described herein). The template nucleic acid molecule may be immobilized to the substrate via a particle (e.g., bead), such as a particle treated according to a method provided herein. The template nucleic acid molecule may be hybridized to a portion of a previously treated nucleic acid molecule of a particle that has been treated as provided herein, which portion may comprise a portion of a growing nucleic acid strand. The substrate may be configured to rotate with respect to a central axis. A solution comprising a plurality of nucleotides or nucleotide analogs may be directed across the array during rotation of the substrate. The plurality of nucleotides or nucleotide analogs may comprise non-terminated nucleotides to facilitate sequencing of homopolymeric regions of a template nucleic acid molecule. The plurality of nucleotides or nucleotide analogs may comprise a plurality of labeled nucleotides or nucleotide analogs labeled with an optically detectable label such as a fluorescent label (e.g., coupled to a nucleotide or nucleotide analog via a linker, such as a semi-rigid linker comprising a cleavable moiety). The plurality of nucleotides or nucleotide analogs may comprise nucleotides or nucleotide analogs of a single canonical type (e.g., adenine, uracil, thymine, cytosine, or guanine-containing nucleotides or nucleotide analogs) or of one or more different types. The template nucleic acid molecule may be subjected to conditions sufficient for nucleotides or nucleotide analogs of the plurality of nucleotides or nucleotide analogs to be incorporated into the growing nucleic acid strand (e.g., in a primer extension reaction). A signal (e.g., an optical signal) indicative of incorporation of a nucleotide or nucleotide analog may be detected (e.g., via optical detection), thereby sequencing the nucleic acid molecule. The plurality of nucleotides or nucleotide analogs may be provided in a first reaction mixture, and provision of the first reaction mixture may be followed by one or more additional flows to wash away unbound nucleotides or nucleotide analogs and reagents, to cleave cleavable moieties of linkers coupling labels to nucleotides or nucleotide analogs, etc. Additional reaction mixtures comprising different combinations of nucleotides or nucleotide analogs may be provided (e.g., in a predefined sequence) to continue sequencing of the template nucleic acid molecule.

Sequencing a nucleic acid molecule (e.g., a nucleic acid molecule immobilized to a particle, which particle may be immobilized to a substrate as described herein) may comprise providing a solution comprising a plurality of optically (e.g., fluorescently) labeled nucleotides, where each optically labeled nucleotide of the plurality of optically labeled nucleotides is of a same type. The solution may also comprise a plurality of non-labeled nucleotides, which non-labeled nucleotides may comprise a nucleobase of the same type as that of the labeled nucleotides. The non-labeled and labeled nucleotides may be included in any useful ratio. For example, at least about 1%, 2%, 2.5%, 5%, 10%, 20%, 30%, 40%, 50%, 60%, 70%, 80%, 90%, 95%, or more nucleotides in the solution may be fluorescently labeled. Alternatively, at most about 1%, 2%, 2.5%, 5%, 10%, 20%, 30%, 40%, 50%, 60%, 70%, 80%, 90%, 95%, or fewer nucleotides in the solution may be fluorescently labeled. Labeled and/or non-labeled nucleotides may be non-terminated such that multiple nucleotides may be incorporated into a growing nucleic acid strand in sequence. A given optically labeled nucleotide of the plurality of fluorescently labeled nucleotides may comprise an optical (e.g., fluorescent) dye that is connected to a nucleotide via a semi-rigid linker, as described herein. The nucleic acid molecule (e.g., nucleic acid molecule coupled to a particle immobilized to a substrate) may be contacted with a primer under conditions sufficient to hybridize the primer to a nucleic acid molecule to be sequenced to generate a sequencing template. The sequencing template may then be contacted with a polymerase and the solution containing the plurality of optically labeled nucleotides, wherein an optically labeled nucleotide of the plurality of optically labeled nucleotides is complementary to the nucleic acid molecule to be sequenced at a position adjacent to the primer. A substrate to which the sequencing template is coupled (e.g., via a particle immobilized to the substrate) may be rotated during provision of the solution such that the solution is radially dispersed across the substrate (e.g., as described herein). One or more optically labeled nucleotides of the plurality of optically labeled nucleotides may thus be incorporated into the sequencing template. One or more non-labeled nucleotides may also be incorporated (e.g., in a homopolymeric sequence). The solution comprising the plurality of optically labeled nucleotides may be washed away from the sequencing template (e.g., using a wash solution). An optical signal emitted by the sequencing template may then be measured. An optical label may be cleaved from an incorporated labeled nucleotide after measuring the optical signal, as described herein. Cleaving an optical label may leave behind a scar (e.g., a residual chemical moiety). A washing flow may be used to remove cleaved labels and other residual materials. One or more additional nucleotide flows, such as one or more additional flows comprising nucleotides containing a same canonical type, may be used to ensure that nucleotides are incorporated into a substantial fraction of available positions. The process may then be repeated with an additional solution comprising additional nucleotides, such as nucleotides of a different type.

In another example, processing of a template nucleic acid molecule may be performed using an open substrate comprising an array of immobilized analytes thereon. For example, a template nucleic acid molecule may be immobilized to the open substrate via a particle (e.g., bead), such as a particle treated according to the methods provided herein. The template nucleic acid molecule may be hybridized to a portion of a previously treated nucleic acid molecule of a particle that has been treated as provided herein, which portion may comprise a portion of a growing nucleic acid strand. The open substrate may be configured to rotate with respect to a central axis. A solution comprising a plurality of probes (e.g., nucleotides or nucleotide analogs) may be delivered to a region proximal to the central axis of the open substrate to introduce the solution to the open substrate. The solution may be dispersed across the open substrate such that at least one of the plurality of probes binds to at least one of the immobilized analytes to form a bound probe. Where the plurality of probes is a plurality of nucleotides or nucleotide analogs, the plurality of nucleotides or nucleotide analogs may comprise non-terminated nucleotides to facilitate sequencing of homopolymeric regions of a template nucleic acid molecule. The plurality of nucleotides or nucleotide analogs may comprise a plurality of labeled nucleotides or nucleotide analogs labeled with a fluorescent label (e.g., coupled to a nucleotide or nucleotide analog via a linker, such as a semi-rigid linker comprising a cleavable moiety). The plurality of nucleotides or nucleotide analogs may comprise nucleotides or nucleotide analogs of a single canonical type (e.g., adenine, uracil, thymine, cytosine, or guanine-containing nucleotides or nucleotide analogs) or of one or more different types. A bound probe may comprise a growing nucleic acid strand having a nucleotide or nucleotide analog incorporated therein. Formation of the bound probe may comprise subjecting a template nucleic acid molecule to conditions sufficient for nucleotides or nucleotide analogs of the plurality of nucleotides or nucleotide analogs to be incorporated into the growing nucleic acid strand (e.g., in a primer extension reaction). A detector may be used to perform a scan of the open substrate. the detector may be an optical detector. The detector may be configured to detect signals (e.g., optical signals) indicative of formation of a bound probe (e.g., incorporation of a nucleotide or nucleotide analog into a growing nucleic acid strand). Accordingly, the detector may be used in sequencing template nucleic acid molecules. The plurality of probes (e.g., nucleotides or nucleotide analogs) may be provided in a first reaction mixture, and provision of the first reaction mixture may be followed by one or more additional flows to wash away unbound probes and reagents, to cleave cleavable moieties of linkers coupling labels to nucleotides or nucleotide analogs, etc. Additional reaction mixtures comprising different combinations of probes (e.g., nucleotides or nucleotide analogs) may be provided (e.g., in a predefined sequence) to, e.g., continue sequencing of the template nucleic acid molecule.

### Sample Processing Systems

A sample processing system may comprise a substrate, and devices and systems that perform one or more operations with or on the substrate, which permit highly efficient dispensing of reagents onto the substrate, and highly efficient imaging of one or more analytes, or signals corresponding thereto, on the substrate, among other operations. The substrate may be an open substrate. The substrate may be substantially planar. The substrate may be textured and/or patterned. In some cases, the texture and/or pattern can distinguish individually addressable locations as described elsewhere herein. The sample processing system may comprise an imaging system comprising a detector. Example substrates and detectors that can be used in the sample processing system are described in further detail in United States Patent Application No. 16/445,798, which is entirely incorporated herein by reference for all purposes.

The systems and methods may utilize a substrate comprising a plurality of individually addressable locations. The plurality of individually addressable locations may be arranged as an array on the substrate. The plurality of individually addressable locations may be otherwise arranged, such as randomly or in any order, on the substrate. Each of the plurality of individually addressable locations, or each of a subset of such locations, may be capable of immobilizing thereto an analyte (e.g., a nucleic acid molecule, a protein molecule, a carbohydrate molecule, etc.) or a reagent (e.g., a nucleic acid molecule, a probe molecule, a barcode molecule, an antibody molecule, a primer molecule, a bead, etc.). For example, an analyte or reagent may be immobilized to an individually addressable location via a support, such as a bead. In an example, a bead is immobilized to the individually addressable location, and the analyte or reagent is immobilized to the bead. In some cases, an individually addressable location may immobilize thereto a plurality of analytes or a plurality of reagents. The plurality of analytes may be copies of a template analyte. For example, the plurality of analytes may have sequence homology or sequence identity. For example, the plurality of analytes may be a clonal amplification colony. In other instances, the plurality of analytes may be different (e.g., comprise different sequences). In one example, the plurality of analytes is immobilized to the individually addressable location via a support, such as a bead. In an example, a bead comprises a plurality of amplification products, as analytes, immobilized thereto, and the bead is immobilized to an individually addressable location on the substrate. In another example, the bead is immobilized to an individually addressable location on the substrate and is configured to capture or bind to a plurality of analytes. In another example, a plurality of reagents is immobilized to an individually addressable location on the substrate via a support, such as a bead. The plurality of reagents may be configured for capturing or binding an analyte or another reagent. The plurality of reagents may be configured for release from the bead. The plurality of reagents bound to the bead may be releasable prior to, during, or subsequent to capturing or binding, or otherwise interacting with, an analyte or another reagent. The substrate may immobilize a plurality of analytes or reagents across multiple individually addressable locations. The plurality of analytes or reagents may be of the same type of analyte or reagent (e.g., a nucleic acid molecule) or may be a combination of different types of analytes or reagents (e.g., nucleic acid molecules, protein molecules, etc.).

Reagents dispensed on the substrate may or may not interact with analytes immobilized on the substrate. For example, when the analytes are nucleic acid molecules and when the reagents comprise nucleotides, the nucleic acid molecules may incorporate or otherwise react with one or more nucleotides. In another example, when the analytes are protein molecules and when the reagents comprise antibodies, the protein molecules may bind to or otherwise react with one or more antibodies. In another example, when the reagents comprise washing reagents, the substrate (and/or analytes on the substrate) may be washed of any unreacted (and/or unbound) reagents, agents, buffers, and/or other particles.

One or more signals (such as optical signals) may be detected from a detection area on the substrate prior to, during, or subsequent to, the dispensing of reagents to generate an output. For example, the output may be an intermediate or final result obtained from processing of the analyte. Signals may be detected in multiple instances. The dispensing of reagents, rotating (or other motion) of the substrate, and/or detecting operations, in any order (independently or simultaneously), may be repeated any number of times to process an analyte. In some instances, the substrate may be washed (e.g., via dispensing washing reagents) between consecutive dispensing of the reagents. One or more detection operations can be performed within a desired time frame. For example, the detection operation can be performed within about 1 minute, 50 seconds, 40 seconds, 30 seconds, 20 seconds, 10 seconds, or less than 10 seconds. In some instances, at least two detection operations can be performed within 1 minute, 50 seconds, 40 seconds, 30 seconds, 20 seconds, 10 seconds, or less than 10 seconds etc. In some instances, at least three detection operations can be performed within 1 minute, 50 seconds, 40 seconds, 30 seconds, 20 seconds, 10 seconds, or less than 10 seconds.

One or more dispensing operations can be performed within a desired time frame. For example, the dispensing operation can be performed within 1 minute, 50 seconds, 40 seconds, 30 seconds, 20 seconds, 10 seconds, or less than 10 seconds. In some instances, at least two dispensing operations can be performed within 1 minute, 50 seconds, 40 seconds, 30 seconds, 20 seconds, 10 seconds, or less than 10 seconds etc. In some instances, at least three dispensing operations can be performed within 1 minute, 50 seconds, 40 seconds, 30 seconds, 20 seconds, 10 seconds, or less than 10 seconds. Any operation or process of one or more methods disclosed herein may be performed within a desired time frame. In some instances, a combination of two or more operations or processes disclosed herein may be performed within a desired time frame. For example, the dispensing operation and the detection method may both be performed within 1 minute, 50 seconds, 40 seconds, 30 seconds, 20 seconds, 10 seconds, or less than 10 seconds. In some instances, at least two dispensing and detection operations can be performed within 1 minute, 50 seconds, 40 seconds, 30 seconds, 20 seconds, 10 seconds, or less than 10 seconds etc. In some instances, at least three dispensing and detection operations can be performed within 1 minute, 50 seconds, 40 seconds, 30 seconds, 20 seconds, 10 seconds, or less than 10 seconds.

A substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), ureaformaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. A substrate may be fully or partially opaque to visible light. In some cases, a substrate may be at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5%, or 100% opaque to visible light. The substrate may have an opacity that is within a range defined by any two of the preceding values. A substrate may be fully or partially transparent to visible light. In some cases, a substrate may be at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5%, or 100% transparent to visible light. The substrate may have a transparency that is within a range defined by any two of the preceding values. In some cases, an illumination power (e.g., a laser power), during detection of a detection area of the substrate, may be adjusted based on the opacity or transparency of the substrate. The one or more layers of the substrate may have a thickness of at least 1 nm, 2 nm, 5 nm, 10 nm, 20 nm, 50 nm, 100 nm, 200 nm, 500 nm, 1 µm, 2 µm, 5 µm, 10 µm, 20 µm, 50 µm, 100 µm, 200 µm, 500 µm, or 1 mm. The one or more layers may have a thickness that is within a range defined by any two of the preceding values. A surface of the substrate may be modified to comprise any of the binders or linkers described herein. A surface of the substrate may be modified to comprise active chemical groups, such as amines, esters, hydroxyls, epoxides, and the like, or a combination thereof. In some instances, such binders, linkers, active chemical groups, and the like may be added as an additional layer or coating to the substrate.

The substrate may have the general form of a cylinder, a cylindrical shell or disk, a rectangular prism, or any other geometric form. The substrate may have a thickness (e.g., a minimum dimension) of at least 100 µm, 200 µm, 500 µm, 1 mm, 2 mm, 5 mm, or 10 mm. The substrate may have a thickness that is within a range defined by any two of the preceding values. The substrate may have a first lateral dimension (such as a width for a substrate having the general form of a rectangular prism or a radius for a substrate having the general form of a cylinder) of at least 1 mm, 2 mm, 5 mm, 10 mm, 20 mm, 50 mm, 100 mm, 200 mm, 500 mm, or 1,000 mm. The substrate may have a first lateral dimension that is within a range defined by any two of the preceding values. The substrate may have a second lateral dimension (such as a length for a substrate having the general form of a rectangular prism) or at least 1 mm, 2 mm, 5 mm, 10 mm, 20 mm, 50 mm, 100 mm, 200 mm, 500 mm, or 1,000 mm. The substrate may have a second lateral dimension that is within a range defined by any two of the preceding values.

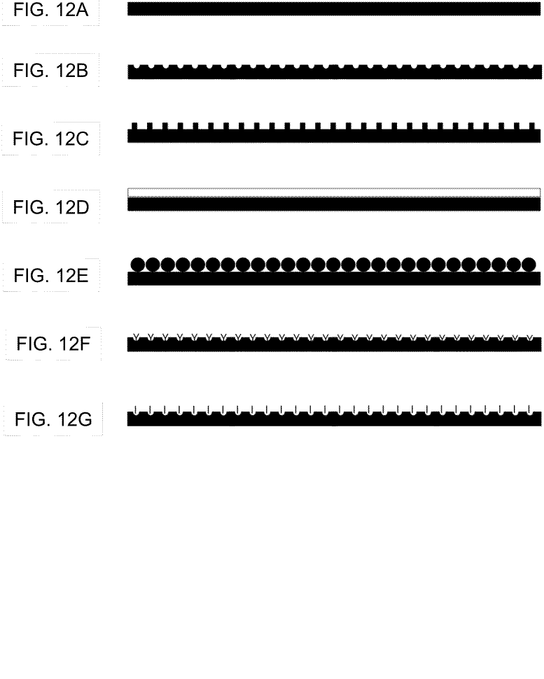

A surface of the substrate may be planar. The surface of the substrate may be substantially planar. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). A surface of the substrate may be uncovered and may be exposed to an atmosphere. Alternatively or in addition, a surface of the substrate may be textured or patterned. For example, the substrate may comprise grooves, troughs, hills, and/or pillars. The substrate may define one or more cavities (e.g., micro-scale cavities or nano-scale cavities). The substrate may define one or more channels. The substrate may have regular textures and/or patterns across the surface of the substrate. For example, the substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. For example, the substrate may have any arbitrary structure above or below a reference level of the substrate. In some instances, a texture of the substrate may comprise structures having a maximum dimension of at most about 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. FIGS. 12A-12G illustrate different examples of cross-sectional surface profiles of a substrate. FIG. 12A illustrates a cross-sectional surface profile of a substrate having a completely planar surface. FIG. 12B illustrates a cross-sectional surface profile of a substrate having semi-spherical troughs or grooves. FIG. 12C illustrates a cross-sectional surface profile of a substrate having pillars, or alternatively or in conjunction, wells. FIG. 12D illustrates a cross-sectional surface profile of a substrate having a coating. FIG. 12E illustrates a cross-sectional surface profile of a substrate having spherical particles. FIG. 12F illustrates a cross-sectional surface profile of FIG. 12B, with a first type of binders seeded or associated with the respective grooves. FIG. 12G illustrates a cross-sectional surface profile of FIG. 12B, with a second type of binders seeded or associated with the respective grooves.

The substrate may comprise an array. For instance, the array may be located on a lateral surface of the substrate. The array may be a planar array. The array may have the general shape of a circle, annulus, rectangle, or any other shape. The array may comprise linear and/or non-linear rows. The array may be evenly spaced or distributed. The array may be arbitrarily spaced or distributed. The array may have regular spacing. The array may have irregular spacing. The array may be a textured array. The array may be a patterned array. The array may comprise a plurality of individually addressable locations. The individually addressable locations may be arranged in any convenient pattern. For example, the individually addressable locations may be randomly oriented on the array. The plurality of individually addressable locations may form separate radial regions around a disk-shaped substrate. The plurality of individually addressable locations may form a square, rectangle, disc, circular, annulus, pentagonal, hexagonal, heptagonal, octagonal, array, or any other pattern. One or more types of individually addressable locations may be generated. The types of individually addressable locations may be arrayed in any useful pattern, such as a square, rectangle, disc, annulus, pentagon, hexagon, radial pattern, etc. In some cases, the two types of individually addressable locations may have different chemical, physical, and/or biological properties (e.g., hydrophobicity, charge, color, topography, size, dimensions, geometry, etc.). For example, a first type of individually addressable location may bind a first type of biological analyte but not a second type of biological analyte, and a second type of individually addressable location may bind the second type of biological analyte but not the first type of biological analyte.

The analyte to be processed may be immobilized to the array. The array may comprise one or more binders described herein, such as one or more physical or chemical linkers or adaptors, that are coupled to a biological analyte. For instance, the array may comprise a linker or adaptor that is coupled to a nucleic acid molecule. Alternatively or in addition, the biological analyte may be coupled to a bead, which bead may be immobilized to the array. In some cases, a subset of the array may not be coupled to a sample or analyte. For example, in substrates that are configured to rotate about a central axis, the samples may not be coupled to a plurality of individually addressable locations of the array located near the central axis. In some cases, the array may be coupled to a sample or an analyte, but not all of the array may be processed. For example, the substrate may be coupled to a sample or analyte (e.g., comprising nucleic acid molecules), but the region of the array that is in proximity to the border of the array may not be subjected to further processing (e.g., detection). Similarly, other reagents may be immobilized to the array.

The individually addressable locations may comprise locations of analytes or groups of analytes that are accessible for manipulation. The manipulation may comprise placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings.

In some cases, the individually addressable locations may be indexed, e.g., spatially, such that the analyte immobilized or coupled to each individually addressable location may be identified from a plurality of analytes immobilized to other individually addressable locations. For example, data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some embodiments, the individually addressable locations are indexed by demarcating part of the substrate. In some embodiments, the surface of the substrate is demarcated using etching. In some embodiments, the surface of the substrate is demarcated using a notch in the surface. In some embodiments, the surface of the substrate is demarcated using a dye or ink. In some embodiments, the surface of the substrate is demarcated by depositing a topographical mark on the surface. In some embodiments, a sample, such as a control nucleic acid sample, may be used to demarcate the surface of the substrate. As will be appreciated, a combination of positive demarcations and negative demarcations (lack thereof) may be used to index the individually addressable locations. In some embodiments, one or more reference objects (e.g., a reference bead that always emits a detectable signal during detection) are immobilized to any location(s) on the substrate, and the individually addressable locations are indexed with reference to the reference object. In some instances, a single reference point or axis (e.g., single demarcation) may be used to index all individually addressable locations. In some embodiments, each of the individually addressable locations is indexed. In some embodiments, a subset of the individually addressable locations is indexed. In some embodiments, the individually addressable locations are not indexed, and a different region of the substrate is indexed.

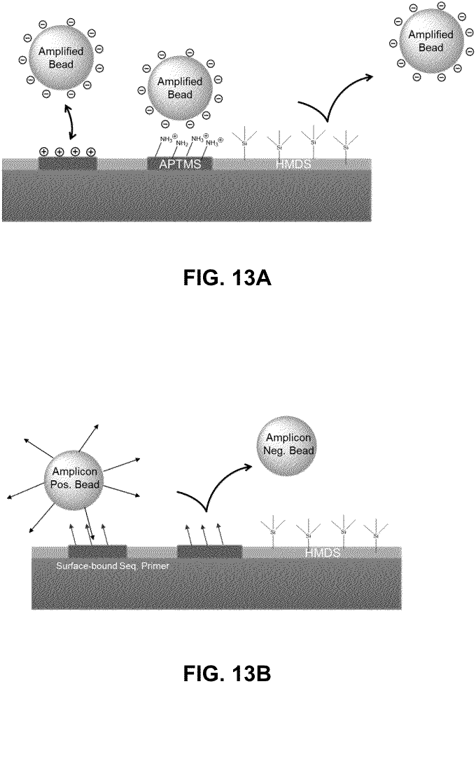

In some cases, an individually addressable location may comprise a distinct surface chemistry. The distinct surface chemistry may distinguish between different addressable locations. The distinct surface chemistry may distinguish between different regions on the substrate. For example, a first location has a first affinity towards an object (e.g., a bead comprising nucleic acid molecules, e.g., amplicons, immobilized thereto) and a second location has a second, different affinity towards the object due to the distinct surface chemistries. The first location and the second location may or may not be located in the same region. The first location and the second location may or may not be disposed on the surface in alternating fashion. In another example, a first region (e.g., comprising a plurality of individually addressable locations) has a first affinity towards an object and a second region has a second, different affinity towards the object due to the distinct surface chemistries. A first location type or region type may comprise a first surface chemistry, and a second location type or region type may comprise a second surface chemistry. In some cases, a third location type or region type may comprise a third surface chemistry. For example, a first location type or region type may comprise a positively charged surface chemistry and/or a hydrophobic surface chemistry, and a second location type or region type may comprise a negatively charged surface chemistry and/or a hydrophilic surface chemistry, as shown in FIG. 13A. The same object (e.g., a bead comprising nucleic acid molecules, e.g., amplicons, immobilized thereto) may have higher affinity towards a first location type or region type compared to a second location type or region type. The same object may be attracted towards a first location type or region type and repelled from a second location type or region type. In other examples, a first location type or region type comprising a first surface chemistry (e.g., a positively charged surface chemistry or a negatively charged surface chemistry) may interact with (e.g., have an affinity towards) a first sample type (e.g., a bead comprising nucleic acid molecules, e.g., amplicons, immobilized thereto) and exclude a second sample type (e.g., a bead lacking nucleic acid molecules, e.g., amplicons, immobilized thereto, e.g., entirely or in substantial volume), for example as illustrated in FIG. 13B. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl)triethoxysilane (APTMS). In some cases, the surface chemistry may comprise a surface primer molecule or any oligonucleotide molecule that has any degree of affinity towards another molecule.

Each individually addressable location may have the general shape or form of a circle, pit, bump, rectangle, or any other shape or form. An individually addressable location of a plurality of locations (e.g., alternating locations) may have an area. In some cases, a location may have an area of about 0.1 square micrometer (µm²), 0.2 µm², 0.25 µm², 0.3 µm², 0.4 µm², 0.5 µm², 0.6 µm², 0.7 µm², 0.8 µm², 0.9 µm², 1 µm², 1.1 µm², 1.2 µm², 1.25 µm², 1.3 µm², 1.4 µm², 1.5 µm², 1.6 µm², 1.7 µm², 1.75 µm², 1.8 µm², 1.9 µm², 2 µm², 2.25 µm², 2.5 µm², 2.75 µm², 3 µm², 3.25 µm², 3.5 µm², 3.75 µm², 4 µm², 4.25 µm², 4.5 µm², 4.75 µm², 5 µm², 5.5 µm², or about 6 µm². A location may have an area that is within a range defined by any two of the preceding values. A location may have an area that is less than about 0.1 µm² or greater than about 6 µm². In some cases, a location may have a width of about 0.1 micrometer (µm), 0.2 µm, 0.25 µm, 0.3 µm, 0.4 µm, 0.5 µm, 0.6 µm, 0.7 µm, 0.8 µm, 0.9 µm, 1 µm, 1.1 µm, 1.2 µm, 1.25 µm, 1.3 µm, 1.4 µm, 1.5 µm, 1.6 µm, 1.7 µm, 1.75 µm, 1.8 µm, 1.9 µm, 2 µm, 2.25 µm, 2.5 µm, 2.75 µm, 3 µm, 3.25 µm, 3.5 µm, 3.75 µm, 4 µm, 4.25 µm, 4.5 µm, 4.75 µm, 5 µm, 5.5 µm, or 6 µm. In some cases, a location may have a width that is within a range defined by any two of the preceding values. A location may have a width that is less than about 0.1 µm or greater than about 6 µm. Each individually addressable location may have a first lateral dimension (such as a radius for individually addressable locations having the general shape of a circle or a width for individually addressable locations having the general shape of a rectangle). In some cases, a first lateral dimension of a location may be at least 1 nm, at least 2 nm, at least 5 nm, at least 10 nm, at least 20 nm, at least 50 nm, at least 100 nm, at least 200 nm, at least 500 nm, at least 1,000 nm, at least 2,000 nm, at least 5,000 nm, or at least 10,000 nm. The first lateral dimension may be within a range defined by any two of the preceding values. Each individually addressable location may have a second lateral dimension (such as a length for individually addressable locations having the general shape of a rectangle). The second lateral dimension may be at least 1 nm, at least 2 nm, at least 5 nm, at least 10 nm, at least 20 nm, at least 50 nm, at least 100 nm, at least 200 nm, at least 500 nm, at least 1,000 nm, at least 2,000 nm, at least 5,000 nm, or at least 10,000 nm. The second lateral dimension may be within a range defined by any two of the preceding values.

In some cases, the locations (e.g., of a same type) may be distributed on a substrate with a pitch determined by the distance between the center of a first location and the center of the closest or neighboring location (e.g., of the same type). Locations may be spaced with a pitch of about 0.1 micrometer (µm), 0.2 µm, 0.25 µm, 0.3 µm, 0.4 µm, 0.5 µm, 0.6 µm, 0.7 µm, 0.8 µm, 0.9 µm, 1 µm, 1.1 µm, 1.2 µm, 1.25 µm, 1.3 µm, 1.4 µm, 1.5 µm, 1.6 µm, 1.7 µm, 1.75 µm, 1.8 µm, 1.9 µm, 2 µm, 2.25 µm, 2.5 µm, 2.75 µm, 3 µm, 3.25 µm, 3.5 µm, 3.75 µm, 4 µm, 4.25 µm, 4.5 µm, 4.75 µm, 5 µm, 5.5 µm, 6 µm, 6.5 µm, 7 µm, 7.5 µm, 8 µm, 8.5 µm, 9 µm, 9.5 µm, or 10 µm. In some case the locations may be positioned with a pitch that is within a range defined by any two of the preceding values. The locations may be positioned with a pitch of less than about 0.1 µm or greater than about 10 µm. In some cases, the pitch between any two locations of the same type may be determined as a function of a size of a loading object (e.g., bead). For example, where the loading object is a bead having a maximum diameter, the pitch may be at least about the maximum diameter of the loading object. In some cases, the center of each of said plurality of individually addressable locations is separated by fewer than about 10 µm, 9 µm, 8 µm, 7 µm, 6 µm, 5 µm, 4 µm, or 3 µm. In some cases, the center of each of said plurality of individually addressable locations is separated by about 2.5 µm, 2.4 µm, 2.3 µm, 2.2 µm, 2.1 µm, 2 µm, 1.9 µm, 1.8 µm, 1.7 µm, 1.6 µm, 1.5 µm, 1.4 µm, 1.3 µm, 1.2 µm, 1.1 µm, 1 µm, 0.9 µm, 0.8 µm, 0.7 µm, 0.6 µm, 0.5 µm, 0.4 µm, 0.3 µm, 0.2 µm, 0.1 µm, or less than about 0.1 µm.

Indexing may be performed using a detection method and may be performed at any convenient or useful step. A substrate that is indexed, e.g., demarcated, may be subjected to detection, such as optical imaging, to locate the indexed locations (e.g., individually addressable locations) and/or the biological analyte. Imaging may be performed using a detection unit. Imaging may be performed using one or more sensors. Imaging may not be performed using the naked eye. The substrate that is indexed may be imaged prior to loading of the biological analyte. Following loading of the biological analyte onto the individually addressable locations, the substrate may be imaged again, e.g. to determine occupancy or to determine the positioning of the biological analyte relative to the substrate. In some cases, the substrate may be imaged after iterative cycles of nucleotide addition (or other probe or other reagent), as described elsewhere herein. The indexing of the substrate and known initial position (individually addressable location) of the biological analyte may allow for analysis and identification of the sequence information for each individually addressable location and/or position. Additionally, spatial indexing may allow for identification of errors that may occur, e.g., sample contamination, sample loss, etc. Example methods for spatial indexing are described in U.S. Pat. No. 16/445,798, which is entirely incorporated herein by reference for all purposes.

The array may be coated with binders. For instance, the array may be randomly coated with binders. Alternatively, the array may be coated with binders arranged in a regular pattern (e.g., in linear arrays, radial arrays, hexagonal arrays etc.). The array may be coated with binders on at least 10%, 20%, 30%, 40%, 50%, 60%, 70%, 80%, 90%, 91%, 92%, 93%, 94%, 95%, 96%, 97%, 98%, or 99% of the number of individually addressable locations, or of the surface area of the substrate. The array may be coated with binders on a fraction of individually addressable locations, or of the surface areas of the substrate, that is within a range defined by any two of the preceding values. The binders may be integral to the array. The binders may be added to the array. For instance, the binders may be added to the array as one or more coating layers on the array.

The binders may be configured to immobilize analytes or reagents, such as through non-specific interactions, such as one or more of hydrophilic interactions, hydrophobic interactions, electrostatic interactions, physical interactions (for instance, adhesion to pillars or settling within wells), and the like. The binders may immobilize analytes or reagents through specific interactions. For instance, where the analyte or reagent is a nucleic acid molecule, the binders may comprise oligonucleotide adaptors configured to bind to the nucleic acid molecule. Alternatively or in addition, such as to bind other types of analytes or reagents, the binders may comprise one or more of antibodies, oligonucleotides, nucleic acid molecules, aptamers, affinity binding proteins, lipids, carbohydrates, and the like. The binders may immobilize analytes or reagents through any possible combination of interactions. For instance, the binders may immobilize nucleic acid molecules through a combination of physical and chemical interactions, through a combination of protein and nucleic acid interactions, etc. The array may comprise an order of magnitude of at least about 10, 100, 10³, 10⁴, 10⁵, 10⁶, 10⁷, 10⁸, 10⁹, 10¹⁰, 10¹¹, or more binders. Alternatively or in addition, the array may comprise an order of magnitude of at most about 10¹¹, 10¹⁰, 10⁹, 10⁸, 10⁷, 10⁶, 10⁵, 10⁴, 10³, 100, 10 or fewer binders. The array may have a number of binders that is within a range defined by any two of the preceding values. In some instances, a single binder may bind a single analyte (e.g., nucleic acid molecule) or single reagent. In some instances, a single binder may bind a plurality of analytes (e.g., plurality of nucleic acid molecules) or a plurality of reagents. In some instances, a plurality of binders may bind a single analyte or a single reagent. Though examples herein describe interactions of binders with nucleic acid molecules, the binders may immobilize other molecules (such as proteins), other particles, cells, viruses, other organisms, or the like. Though examples herein describe interactions of binders with samples or analytes, the binders may similarly immobilize reagents.

In some instances, each location, or a subset of such locations, may have immobilized thereto an analyte (e.g., a nucleic acid molecule, a protein molecule, a carbohydrate molecule, etc.) or reagent. In other instances, a fraction of the plurality of individually addressable locations may have immobilized thereto an analyte or reagent. A plurality of analytes or reagents immobilized to the substrate may be copies of a template analyte or template reagent. For example, the plurality of analytes (e.g., nucleic acid molecules) or reagents may have sequence homology. In other instances, the plurality of analytes or reagents immobilized to the substrate may not be copies. The plurality of analytes may be of the same type of analyte (e.g., a nucleic acid molecule) or reagent or may be a combination of different types of analytes or reagents (e.g., nucleic acid molecules, protein molecules, etc.).

In some instances, the array may comprise a plurality of types of binders. For example, the array may comprise different types of binders to bind different types of analytes or reagents. For example, the array may comprise a first type of binders (e.g., oligonucleotides) configured to bind a first type of analyte (e.g., nucleic acid molecules) or reagent, and a second type of binders (e.g., antibodies) configured to bind a second type of analyte (e.g., proteins) or reagent, and the like. In another example, the array may comprise a first type of binders (e.g., first type of oligonucleotide molecules) to bind a first type of nucleic acid molecules and a second type of binders (e.g., second type of oligonucleotide molecules) to bind a second type of nucleic acid molecules, and the like. For example, the substrate may be configured to bind different types of analytes or reagents in certain fractions or specific locations on the substrate by having the different types of binders in the certain fractions or specific locations on the substrate.

An array may have any number of individually addressable locations. For instance,the array may have at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, 1,000,000,000, 2,000,000,000, 5,000,000,000, 10,000,000,000, 20,000,000,000, 50,000,000,000, or 100,000,000,000 individually addressable locations. The array may have a number of individually addressable locations that is within a range defined by any two of the preceding values. Each individually addressable location may be digitally and/or physically accessible individually (from the plurality of individually addressable locations). For example, each individually addressable location may be located, identified, and/or accessed electronically or digitally for mapping, sensing, associating with a device (e.g., detector, processor, dispenser, etc.), or otherwise processing. As described elsewhere herein, each individually addressable location may be indexed. Alternatively, the substrate may be indexed such that each individually addressable location may be identified during at least one step of the process. Alternatively or in addition, each individually addressable location may be located, identified, and/or accessed physically, such as for physical manipulation or extraction of an analyte, reagent, particle, or other component located at an individually addressable location. In some instances, each individually addressable locations may have or be coupled to a binder, as described herein, to immobilize an analyte thereto. In some instances, only a fraction of the individually addressable locations may have or be coupled to a binder. In some instances, an individually addressable location may have or be coupled to a plurality of binders to immobilize an analyte or reagent thereto.

The analytes bound to the individually addressable locations may include, but are not limited to, molecules, cells, tissues, organisms, nucleic acid molecules, nucleic acid colonies, beads, clusters, polonies, DNA nanoballs, or any combination thereof (e.g., bead having attached thereto one or more nucleic acid molecules, e.g., one or more clonal populations of nucleic acid molecules). The analytes bound to the individually addressable locations may include any analyte described herein. The bound analytes may be immobilized to the array in a regular, patterned, periodic, random, or pseudo-random configuration, or any other spatial arrangement. In some embodiments, the analytes are bound to bead(s) which may then associate with or be immobilized to the substrate or regions of the substrate (e.g., individually addressable locations). In some embodiments, the analytes comprise a bead or a plurality of beads. In some cases, the bead or plurality of beads may comprise another analyte (e.g., nucleic acid molecule) or a clonal population of other analytes (e.g., a nucleic acid molecule that has been amplified on the bead). Such other analytes may be attached or otherwise coupled to the bead. For example, an analyte may comprise a plurality of beads, each bead having a clonal population of nucleic acid molecules attached thereto. In some cases, the bead is magnetic, and application of a magnetic field or using a magnet may be used to direct the analytes or beads comprising the analytes to the individually addressable locations. In some cases, the bead is electrically charged, and application of an electric field may be used to direct the analytes or beads comprising the analytes to the individually addressable locations. In some cases, a fluid may be used to direct the analyte to the individually addressable locations. The fluid may be a ferrofluid, and a magnet may be used to direct the fluid to the individually addressable locations. The individually addressable locations may alternatively or in conjunction comprise a material that is sensitive to a stimulus, e.g., thermal, chemical, or electrical or magnetic stimulus. For example, the individually addressable locations may comprise a photo-sensitive polymer or reagent that is activated when exposed to electromagnetic radiation. In some cases, a caged molecule may be used to reveal binding (e.g., biotin) moieties (e.g., binders) on the substrate. Subsequent exposure to a particular wavelength of light may result in un-caging of the binding moieties. A bead, e.g., with streptavidin, comprising the analyte may then associate with the uncaged binding moieties. In some cases, a subset of the individually addressable locations may not contain beads. In such cases, blank beads may be added to the substrate. The blank beads may then occupy the regions that are unoccupied by an analyte. In some cases, the blank beads have a higher binding affinity or avidity for the individually addressable locations than the beads comprising the analyte. In some cases, unoccupied locations, or binders at such locations, may be destroyed or rendered inactive. In some cases, unoccupied locations may be subjected to a process to remove any unbound analyte, e.g., aspiration, washing, air blasting etc. In some cases, the sample comprising the analyte may be loaded onto the substrate using a device, e.g., a microfluidic device, closed flow cell, etc. The loaded analyte may then associate with or be immobilized to the substrate or the individually addressable locations of the substrate. In such cases, the device may be removed following loading of the sample. Though examples herein describe immobilization of analytes to the substrate, similar mechanisms may immobilize reagents to the substrate. For example, reagents may comprise or be coupled to bead(s).

An analyte may be bound to any number of beads. Different analytes may be bound to any number of beads. The beads may be unique (i.e., distinct from each other). Any number of unique beads may be used. For instance, an order of magnitude of at least about 10, 100, 1000, 10,000, 100,000, 1,000,000, 10,000,000, 100,000,000, 1,000,000,000, 10,000,000,000, 100,000,000,000 or more different beads may be used. Alternatively or in addition, an order of magnitude of at most about 100,000,000,000, 10,000,000,000, 1,000,000,000, 100,000,000, 10,000,000, 1,000,000, 100,000, 10,000, 1000, 100, or 10 different beads may be used. A number of different beads can be within a range defined by any two of the preceding values. The beads may be distinguishable from one another using a property of the beads, such as color, reflectance, anisotropy, brightness, fluorescence, etc. As described elsewhere herein, in some cases, different beads may comprise different tags (e.g., nucleic acid sequences) coupled thereto. For example, a bead may comprise an oligonucleotide molecule comprising a tag that identifies a bead amongst a plurality of beads.

A sample may be diluted such that the approximate occupancy of the individually addressable locations is controlled. A sample may be diluted at least to a dilution of 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900, 1:1000, 1:10000, 1:100000, 1:1000000, 1:10000000, or 1:100000000. Alternatively, a sample may be diluted at most to a dilution of 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900, 1:1000, 1:10000, 1:100000, 1:1000000, 1:10000000, or 1:100000000. A dilution between any of these dilution values may also be used.

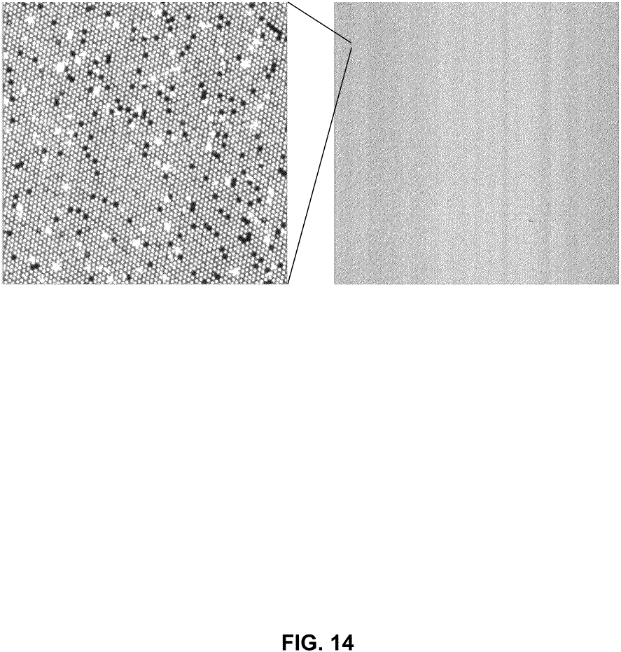

In some instances, a sample may comprise beads. Beads may be dispersed on a surface in any pattern, or randomly. Beads may be dispersed on one or more regions (e.g., a region having a particular surface chemistry) of a surface. In some cases, beads may be dispersed on a surface or a region of a surface in a hexagonal lattice, as shown in FIG. 14, which illustrates in the right panel a zoomed out image of a portion of a surface, and in the left panel a zoomed in image of a section of the portion of the surface. In some instances, a sample comprising beads may be dispersed on a surface comprising distinct locations/regions differentiated by surface chemistry (e.g., as illustrated in FIG. 13A and FIG. 13B). For example, a sample comprising beads may be dispensed on a surface comprising positively charged locations/regions and/or hydrophobic locations/regions. The beads may have a high affinity for a first location type or region type (e.g., positively charged). The beads may have a low affinity for a second location type or region type (e.g., hydrophobic). A location may comprise no more than 1, 2, 3, 4, 5, 6, 7, 8, 9, or 10 beads per location. In some embodiments, a bead may be substantially centered within an individually addressable location when immobilized. A location may have a width that is up to about 0.5 times, 0.6 times, 0.7 times, 0.8 times, 0.9 times, 1 times, 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, or 3 times the diameter (e.g., maximum diameter) of the bead. In some embodiments, a region may be spaced with a pitch determined by the distance between the center of a first location and the center of the closest or neighboring location of the same type. A location may be spaced with a pitch that is at least about 1 times, 1.2 times, 1.4 times, 1.6 times, 1.8 times, 2 times, 2.2 times, 2.4 times, 2.6 times, 2.8 times, 3 times, 3.2 times, 3.4 times, 3.6 times, 3.8 times, 4 times, 4.2 times, 4.4 times, 4.6 times, 4.8 times, or 5 times the diameter (e.g.,. maximum diameter) of the bead. In some cases, one or more of a location size, a location spacing, a bead affinity, a location surface chemistry may be adjusted to reduce a deviation of a bead contact point from the center of a region. Though examples herein describe a sample comprising beads, similarly, a reagent dispensed to the substrate may comprise beads.

A surface comprising a plurality of individually addressable locations may be loaded with beads. The beads may be loaded onto the surface at an occupancy determined by the number of locations of a given location type comprising at least one bead out of the total number of locations of the same location type. A surface comprising a plurality of locations may have occupancy of at least about 50%, 60%, 70%, 80%, 85%, 86%, 87%, 88%, 89%, 90%, 91%, 92%, 93%, 94%, 95%, 96%, 97%, 98%, 99.5%, or up to about 100%. For example, a surface may have at least about 90% of the locations of a given location type loaded with at least one bead. Beads may land on the surface with a landing efficiency determined by the number of beads that bind to the surface out of the total number of beads dispensed on the surface. Beads may be dispensed onto a surface with a landing efficiency of at least about 10%, 20%, 30%, 40%, 50%, 55%, 60%, 65%, 70%, 75%, 80%, 90%, up to about 100%. In some embodiments, one or more of a temperature, an incubation time, a surfactant, or a salt concentration of a solution comprising beads may be adjusted to increase bead occupancy. In some embodiments, one or more of a temperature, an incubation time, a surfactant, or a salt concentration of a solution comprising beads may be adjusted to increase bead loading efficiency.

In some cases, at least about 5%, 10%, 15%, 20%, 25%, 30%, 35%, 40%, 45%, 50%, 55%, 60%, 65%, 70%, 75%, 80%, 85%, 90%, or 95% of the available surface area of a substrate may be configured to accept a bead. Where less than 100% of the available surface area loads thereon a bead (e.g., have a bead immobilized thereto), the negative space (e.g., locations in which there is no bead) may be used as a reference to identify and/or index different individually addressable locations of the positive space (e.g., locations in which there is a bead). In an example, a single individually addressable location acting in negative space is sufficient to index the entire substrate. In such an example, the single individually addressable location will always remain ‘dark’ during imaging, such as during sequencing, as opposed to other individually addressable locations in the positive space which will light up (e.g., fluoresce) at different points in time, due to the analyte or reagent in the positive space, such that the single individually addressable location which is always ‘dark’ may act as a reference against all other individually addressable locations. In other examples, multiple individually addressable locations acting in negative space may facilitate indexing of the substrate. Alternatively or in addition, a reference bead which is always ‘bright’ (e.g., always fluorescing regardless of time point) may be used as a reference to identify and/or index different individually addressable locations of the positive space. In such cases, even with 100% or substantially 100% of the available surface area loaded with beads, including the reference bead, the different individually addressable locations may be identified and/or indexed.

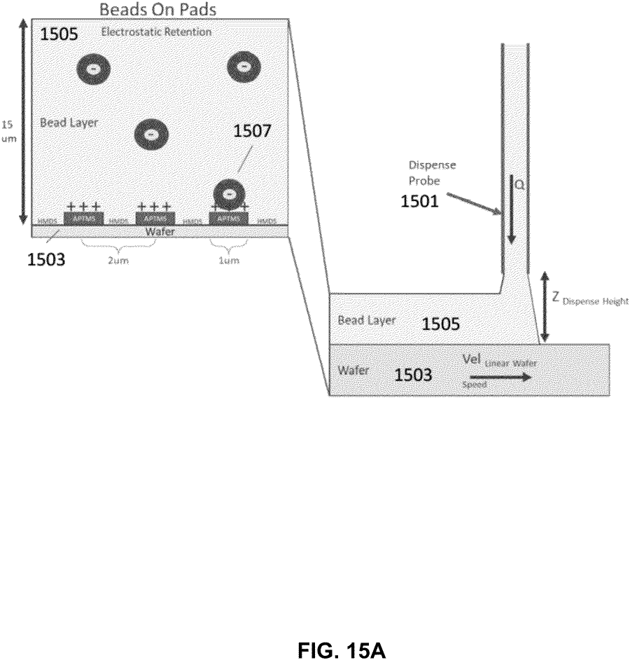

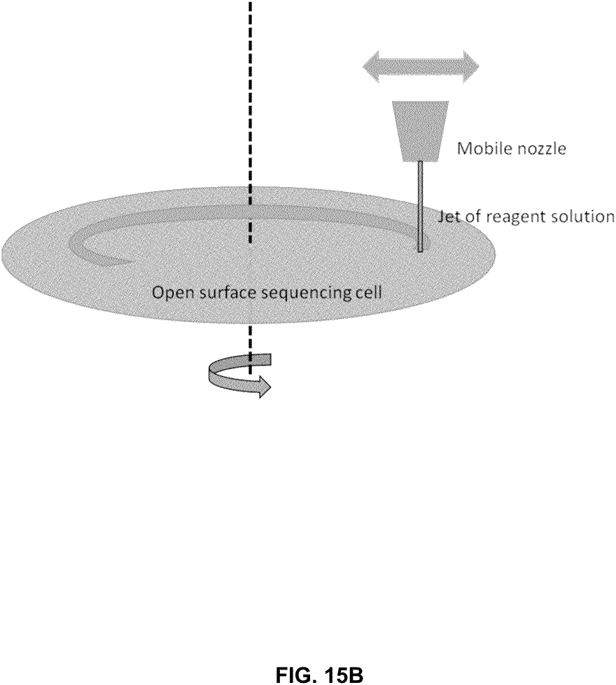

In some cases, beads may be dispensed to the substrate according to one or more systems and methods shown in FIGS. 15A-15B. As shown in FIG. 15A, a solution comprising beads may be dispensed from a dispense probe 1501 (e.g., a nozzle) to a substrate 1503 (e.g., a wafer) to form a layer 1505. (e.g., a bead layer). The dispense probe may be positioned at a fixed height (“Z”) above the substrate., which is the maximum height of layer 1505. In the illustrated example, the beads are retained in the layer 1505 by electrostatic retention and may immobilize to the substrate at respective individually addressable locations. A set of beads in the solution may each comprise a population of amplified products (e.g., nucleic acid molecules) immobilized thereto, which amplified products accumulate to a negative charge on the bead with affinity to a positive charge. Otherwise, the beads may comprise reagents that have a negative charge. The substrate comprises alternating surface chemistry between distinguishable locations, in which a first location type comprises APTMS carrying a positive charge with affinity towards the negative charge of the amplified bead (e.g., a bead comprising amplified products immobilized thereto, and as distinguished from a negative bead which does not the comprise the same) or other bead comprising the negative charge, and a second location type comprises HMDS which has lower affinity and/or is repellant of the amplified bead or other bead comprising the negative charge. Within the layer 1505 comprising the dispensed bead layer 1505 comprising the dispensed beads, a bead (e.g., a bead having immobilized thereto a population of amplified product) may successfully land on a first location of the first location type (e.g., bead immobilized to first location of the first location type 1507). In the illustrated example, the location size is about 1 micrometer, the pitch between the different locations of the same location type (e.g., first location type) is about 2 micrometers, and the bead layer has a depth of 15 micrometers.

FIG. 15B illustrates a solution (e.g., reagent and/or beads) being dispensed along a path on an open surface of the substrate. As shown in FIG. 15B, a reagent solution may be dispensed from a dispense probe (e.g., a nozzle) onto a substrate. In some embodiments, a solution may be dispensed from a plurality of dispense probes. For example, a first reagent in a solution may be dispensed from a first dispense probe, a second reagent in a solution may be dispensed from a second dispense probe, and a third reagent in a solution may be dispensed from a third dispense probe. In some cases, the reagents dispensed from different dispense probes may combine on the substrate to form a homogenous or substantially homogenous solution. The dispense probe may be positioned at a fixed height above a substrate (e.g., a wafer). The substrate and the dispense probe may move in any configuration with respect to each other to achieve any pattern (e.g., linear pattern, substantially spiral pattern, etc.) of reagent dispensing on the substrate. A solution may be provided to the substrate prior to or during motion of the substrate to disperse the solution across the array on the substrate.

In some cases, the solution may be dispensed on the substrate while the substrate is stationary; the substrate may then be subjected to motion (e.g., rotation) following the dispensing of the solution. Alternatively, the substrate may be subjected to motion prior to the dispensing of the solution; the solution may then be dispensed on the substrate while the substrate is moving. The solution may be dispensed in a manner so as to provide a substantially uniform layer across the substrate. Motion of the substrate may be selected to attain a desired thickness of a film of the solution on the substrate. Alternatively or in combination, the viscosity of the solution may be chosen to attain a desired thickness of a film of the solution on the substrate. Alternatively or in combination, a dispense flow rate of the solution may be chosen to attain a desired thickness of a film of the solution on the substrate. For instance, one or more conditions may be applied to attain a film thickness of at least 10 nm, 20 nm, 50 nm, 100 nm, 200 nm, 500 nm, 1 µm, 2 µm, 5 µm, 10 µm, 20 µm, 50 µm, 100 µm. 200 µm, 500 µm, or 1 mm, or more. Alternatively or in addition, one or more conditions may be applied to attain a film thickness of at most 10 nm, 20 nm, 50 nm, 100 nm, 200 nm, 500 nm, 1 µm, 2 µm, 5 µm, 10 µm, 20 µm, 50 µm, 100 µm, 200 µm, 500 µm, or 1 mm, or less than 10 nanometers. One or more conditions may be applied to attain a film thickness that is within a range defined by any two of the preceding values. The thickness of the film may be measured or monitored. Measurements or monitoring of the thickness of the film may be incorporated into a feedback system to better control the film thickness. The thickness of the film may be measured or monitored by a variety of techniques. For instances, the thickness of the film may be measured or monitored by thin film spectroscopy with a thin film spectrometer, such as a fiber spectrometer.

In some cases, the solution may be heated prior to being dispensed on the substrate. The solution may be at a higher temperature than the ambient temperature. The solution may be heated to about 25° C., 26° C., 27° C., 28° C., 29° C., 30° C., 31° C., 32° C., 33° C., 34° C., 35° C., 36° C., 37° C., 38° C., 39° C., 40° C., 45° C., about 50° C., 55° C., 60° C., 65° C., 70° C., 75° C., 80° C., 85° C., 90° C., or 95° C. prior to dispensing. In some cases, a solution may be heated to a temperature that is within a range defined by any two of the preceding values.

One or more solutions or reagents may be delivered to a substrate by any of the delivery methods disclosed herein. Non-limiting solution delivery methods and systems are described, for example, in U.S Pat. Application No. 16/445,798, which is hereby incorporated by reference in its entirety. In some embodiments, two or more solutions or reagents are delivered to the substrate using the same delivery method. In some embodiments, two or more solutions are delivered to the substrate such that the time between contacting a solution or reagent and a subsequent solution or reagent is substantially similar for each region of the substrate contacted to the one or more solutions or reagents. In some embodiments, a solution or reagent may be delivered as a single mixture. In some embodiments, dispensing of each component of the two or more components may be temporally separated. One or more solutions or reagents dispensed on a surface may undergo a reaction on the surface. For example, a first solution (e.g., comprising a reactant) dispensed on the surface may react with a second solution (e.g., comprising an enzyme) dispensed on the surface on top of the first solution. One or more solutions dispensed on a surface may deactivate or quench a chemical reaction. For example, a quenching solution (e.g., comprising EDTA or an acid) may be added to the substrate on top of a reaction to quench the reaction. In some embodiments, a quenching solution is dispensed on the surface in the same pattern as a solution comprising a reactant, thereby maintaining a substantially constant reaction time at each region of the surface to which a solution is dispensed. In some embodiments, a quenching solution is dispensed on the surface in the same pattern as a solution comprising an enzyme, thereby maintaining a substantially constant reaction time at each region of the surface to which a solution is dispensed. Alternatively or in addition, similarly, one or more solutions dispensed on a surface may activate or catalyze a chemical reaction. For example, an activating solution (e.g., comprising catalysts, enzymes, primers, etc.) may be added to the substrate on top of a reaction (e.g., in the same dispense pattern) to activate or catalyze a reaction.

A variety of methods may be employed to dispense one or more solutions onto a substrate to ensure a substantially similar reaction time across an area of the substrate in contact with the one or more solutions. In some embodiments, a solution may be spin-coated onto a surface by dispensing the solution at or near the axis of rotation of a rotating substrate such that the centrifugal force of the rotating substrate facilitates the outward spread of the solution away from the axis of rotation. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution. Additional methods of solution delivery to a substrate known in the art may be used.

In some embodiments, a solution may be dispensed onto a substrate using the method illustrated in FIG. 15B, where a jet of a solution may be dispensed from a nozzle to a rotating substrate. The nozzle may translate radially relative to the rotating substrate, thereby dispensing the solution in a spiral pattern onto the substrate.

A solution may be incubated on the substrate. In some embodiments, the solution may be incubated on the substrate under conditions that maintain a layer of fluid on the surface. The solution may be incubated for at least about 5, 10, 15, 20, 25, 30, 35, 40, 45, 50, 55, 60, 65, 70, 75, 80, 85, or 90 minutes. In some cases the incubation time may be within a range defined by any two of the preceding values. In some cases, the incubation may be for more than 90 minutes. In some instances, the layer of fluid may maintain a film thickness of at least 10 nm, 20 nm, 50 nm, 100 nm, 200 nm, 500 nm, 1 µm, 2 µm, 5 µm, 10 µm, 20 µm, 50 µm, 100 µm. 200 µm, 500 µm, or 1 mm during incubation. One or more of the temperature of the chamber, the humidity of the chamber, the rotation of the substrate, or the composition of the fluid may be adjusted such that the layer of fluid is maintained during incubation.

The substrate or a surface thereof may comprise other features that aid in solution or reagent retention on the substrate or thickness uniformity of the solution or reagent on the substrate. In some cases, the surface may comprise a raised edge (e.g., a rim) which may be used to retain solution on the surface. The surface may comprise a rim near the outer edge of the surface, thereby reducing the amount of the solution that flows over the outer edge.

The solution may comprise any sample or any analyte disclosed herein. The solution may comprise any reagent disclosed herein. In some cases, the solution may be a reaction mixture comprising a variety of components. For example, the solution may comprise a plurality of probes configured to interact with the analyte. For example, the probes may have binding specificity to the analyte. In another example, the probes may not have binding specificity to the analyte. A probe may be configured to permanently couple to the analyte. A probe may be configured to transiently couple to the analyte. For example, a nucleotide probe may be permanently incorporated into a growing strand hybridized to a nucleic acid molecule analyte. Alternatively, a nucleotide probe may transiently bind to the nucleic acid molecule analyte. Transiently coupled probes may be subsequently removed from the analyte. Subsequent removal of the transiently coupled probes from an analyte may or may not leave a residue (e.g., chemical residue) on the analyte. A type of probe in the solution may depend on the type of analyte. A probe may comprise a functional group or moiety configured to perform specific functions. For example, a probe may comprise a label (e.g., dye). A probe may be configured to generate a detectable signal (e.g., optical signal), such as via the label, upon coupling or otherwise interacting with the analyte. In some instances, a probe may be configured to generate a detectable signal upon activation (e.g., application of a stimulus). In another example, a nucleotide probe may comprise reversible terminators (e.g., blocking groups) configured to terminate polymerase reactions (until unblocked). The solution may comprise other components to aid, accelerate, or decelerate a reaction between the probe and the analyte (e.g., enzymes, catalysts, buffers, saline solutions, chelating agents, reducing agents, other agents, etc.). In some instances, the solution may be a washing solution. In some instances, a washing solution may be directed to the substrate to bring the washing solution in contact with the array after a reaction or interaction between reagents (e.g., a probe) in a reaction mixture solution with an analyte immobilized on the array. The washing solution may wash away any free reagents from the previous reaction mixture solution. In some instances, the solution may comprise a cleaving agent, such as to cleave a label and/or a blocking group, and/or otherwise act on a cleavage site (e.g., to cleave a sequence). Though examples herein describe interaction between a probe and an analyte, the probe may be configured to interact with any other reagent described herein, for example a reagent immobilized to an individually addressable location. In some examples, an analyte in one processing experiment may be used as a reagent for another processing experiment. The different processing experiments may be performed on the same substrate or different substrates. In an example, a bead comprising an oligonucleotide molecule comprising a barcode sequence may be immobilized to an individually addressable location on the substrate, the oligonucleotide molecule may be interrogated as the analyte by one or more probes such as to identify the barcode sequence, such as to index the individually addressable location with the barcode sequence, a sample may be loaded onto the substrate, such as over the bead, and then the bead comprising the oligonucleotide molecule immobilized to the individually addressable location used to capture another analyte (e.g., nucleic acid molecule, e.g., mRNA transcript) at the individually addressable location such as to tag the other analyte with the barcode sequence. The tagged analyte may be collected from the substrate, processed (e.g., released from the bead and amplified on another bead such that the other bead comprises an amplification product of the tagged analyte), and it or its derivative reloaded onto another substrate for interrogation by one or more probes such as to determine a sequence of the tagged analyte or its derivative.

A detectable signal, such as an optical signal (e.g., fluorescent signal), may be generated upon reaction between a probe in the solution and the analyte. For example, the signal may originate from the probe and/or the analyte. The detectable signal may be indicative of a reaction or interaction between the probe and the analyte. The detectable signal may be a non-optical signal. For example, the detectable signal may be an electronic signal. The detectable signal may be detected by one or more sensors. For example, an optical signal may be detected via one or more optical detectors in an optical detection scheme described elsewhere herein. The signal may be detected during rotation of the substrate. The signal may be detected following termination of the rotation. The signal may be detected while the analyte is in fluid contact with the solution. The signal may be detected following washing of the solution. In some instances, after the detection, the signal may be muted, such as by cleaving a label from the probe and/or the analyte, and/or modifying the probe and/or the analyte. Such cleaving and/or modification may be affected by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of directing a solution to the substrate and detection of one or more signals indicative of a reaction between a probe in the solution and an analyte in the array may be repeated one or more times. Such operations may be repeated in an iterative manner. For example, the same analyte immobilized to a given location in the array may interact with multiple solutions in the multiple repetition cycles. For each iteration, the additional signals detected may provide incremental, or final, data about the analyte during the processing. For example, where the analyte is a nucleic acid molecule and the processing is sequencing, additional signals detected for each iteration may be indicative of a base in the nucleic acid sequence of the nucleic acid molecule. The operations may be repeated at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, or 1,000,000,000 cycles to process the analyte. In some instances, a different solution may be directed to the substrate for each cycle. For example, at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, or 1,000,000,000 solutions may be directed to the substrate.

In some instances, a washing solution may be directed to the substrate between each cycle (or at least once during each cycle). For instance, a washing solution may be directed to the substrate after each type of reaction mixture solution (e.g., each type of nucleotide) is directed to the substrate. The washing solutions may be distinct. The washing solutions may be identical. The washing solution may be dispensed in any manner as described herein. In some instances, at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, or 1,000,000,000 washing solutions may be directed to the substrate.

### Sample Processing Using an Array

In some instances, a method for sequencing may employ sequencing by synthesis schemes wherein a nucleic acid molecule is sequenced base-by-base with primer extension reactions. For example, a method for sequencing a nucleic acid molecule may comprise providing a substrate comprising an array having immobilized thereto the nucleic acid molecule. The array may be a planar array. The method may comprise directing a solution comprising a plurality of nucleotides across the array prior to or during motion of the substrate. The nucleic acid molecule may be subjected to a primer extension reaction under conditions sufficient to incorporate or specifically bind at least one nucleotide from the plurality of nucleotides into a growing strand that is complementary to the nucleic acid molecule. A signal indicative of incorporation or binding of at least one nucleotide may be detected, thereby sequencing the nucleic acid molecule.

In some instances, the method may comprise, prior to providing the substrate having immobilized thereto the nucleic acid molecule, immobilizing the nucleic acid molecule to the substrate. For example, a solution comprising a plurality of nucleic acid molecules comprising the nucleic acid molecule may be directed to the substrate prior to, during, or subsequent to rotation of the substrate, and the substrate may be subject to conditions sufficient to immobilize at least a subset of the plurality of nucleic acid molecules as an array on the substrate.

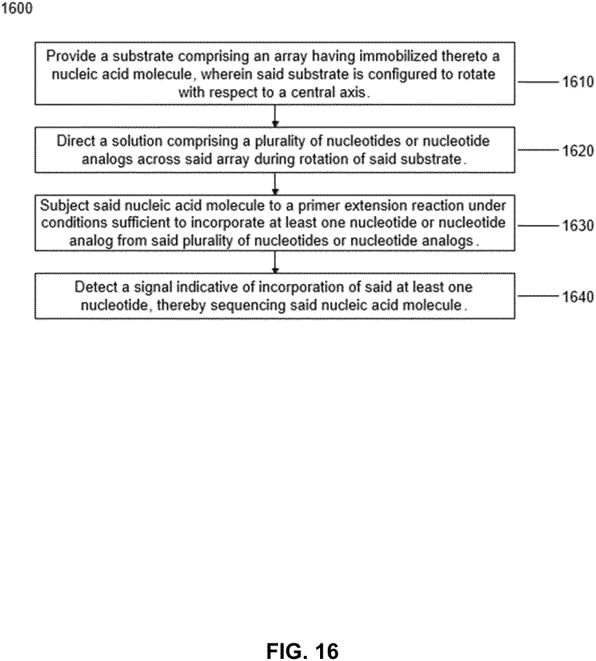

FIG. 16 shows a flowchart for an exemplary method 1600 for sequencing a nucleic acid molecule. In a first operation 1610, the method may comprise providing a substrate, as described elsewhere herein. The substrate may comprise an array of a plurality of individually addressable locations. The array may be a planar array. The array may be a textured array. The array may be a patterned array. For example, the array may define individually addressable locations with wells and/or pillars. A plurality of nucleic acid molecules, which may or may not be copies of the same nucleic acid molecule, may be immobilized to the array. Each nucleic acid molecule from the plurality of nucleic acid molecules may be immobilized to the array at a given individually addressable location of the plurality of individually addressable locations. The substrate may be configured to rotate with respect to an axis. The axis may be an axis through the center or substantially center of the substrate. The axis may be an off-center axis. The substrate may be configured to rotate (or otherwise move as described herein) with different rotational velocities during different operations described herein.

In a second operation 1620, the method may comprise directing a solution across the array prior to or during motion of the substrate in any method as described herein. In some cases, a solution may comprise beads, as described elsewhere herein. The beads may be coated with a nucleic acid molecule to be sequenced. The solution comprising beads may be dispensed onto the substrate using the methods described herein. For example, the solution comprising beads may be dispensed onto the substrate, as illustrated in FIGS. 15A and 15B. The beads may be dispensed according to any pattern (e.g., a spiral pattern). In some cases, the beads may preferentially interact with a first region type of the substrate (e.g., a positively charged region), as illustrated in FIG. 15A. In some cases, a bead may not interact with a second region type of the substrate (e.g., a hydrophobic region). In some cases, a bead coated with a nucleic acid molecule may interact with a first region of the substrate (e.g., a positively charged region), and a bead that is not coated with a nucleic acid molecule may not interact with the first region type of the substrate (e.g., FIG. 15B).

In some instances, the solution may comprise probes configured to interact with nucleic acid molecules. For example, in some instances, such as for performing sequencing by synthesis, the solution may comprise a plurality of nucleotides (in single bases). The plurality of nucleotides may include nucleotide analogs, naturally occurring nucleotides, and/or non-naturally occurring nucleotides, as described herein. The plurality of nucleic acid molecules may comprise nucleotide analogs, naturally occurring nucleotides, non-naturally occurring nucleotides, or any combination thereof. The plurality of nucleotides may or may not be bases of the same canonical base type (e.g., A, T, G, C, etc.). For example, the solution may or may not comprise bases of only one type. The solution may comprise at least 1 type of base or bases of at least 2, 3, 4, 5, 6, 7, 8, 9, or at least 10 types. For instance, the solution may comprise any possible mixture of A, T, C, and G, or subset thereof. In some instances, the solution may comprise a plurality of natural nucleotides and non-natural nucleotides. The plurality of natural nucleotides and non-natural nucleotides may or may not be bases of the same type. In some cases, the solution may comprise probes that are oligomeric (e.g., oligonucleotide primers). For example, in some instances, such as for performing sequencing by synthesis, the solution may comprise a plurality of nucleic acid molecules, e.g., primers, that comprise 1, 2, 3, 4, 5, 6, 7, 8, 9, 10, 20, 30, 40, 50, 60, 70, 80, 90, 100 or more nucleotide bases.

In some cases, the plurality of nucleotides in the solution may be non-terminated. In some cases, none of the nucleotides in the solution may be terminated. Following incorporation of a non-terminated nucleotide into a nucleic acid strand, the nucleic acid strand may be able to incorporate another nucleotide. For example, where a solution of non-terminated A-base nucleotides are provided to a template that comprises a poly-T sequence, at the poly-T sequence locations the nucleic acid strand may incorporate multiple non-terminated A-base nucleotides in consecution.

Alternatively, one or more nucleotides of the plurality of nucleotides may be terminated (e.g., reversibly terminated). For example, a nucleotide may comprise a reversible terminator, or a moiety that is capable of terminating primer extension reversibly. Nucleotides comprising reversible terminators may be accepted by polymerases and incorporated into growing nucleic acid sequences analogously to non-reversibly terminated nucleotides. Following incorporation of a nucleotide analog comprising a reversible terminator into a nucleic acid strand, the reversible terminator may be removed to permit further extension of the nucleic acid strand. A reversible terminator may comprise a blocking or capping group that is attached to the 3′-oxygen atom of a sugar moiety (e.g., a pentose) of a nucleotide or nucleotide analog. Such moieties are referred to as 3′-O-blocked reversible terminators. Examples of 3′-O-blocked reversible terminators include, for example, 3′-ONH₂ reversible terminators, 3′-O-allyl reversible terminators, and 3′-O-aziomethyl reversible terminators. Alternatively, a reversible terminator may comprise a blocking group in a linker (e.g., a cleavable linker) and/or dye moiety of a nucleotide analog. 3′-unblocked reversible terminators may be attached to both the base of the nucleotide analog as well as a fluorescing group (e.g., label, as described herein). Examples of 3′-unblocked reversible terminators include, for example, the “virtual terminator” developed by Helicos BioSciences Corp. and the “lightning terminator” developed by Michael L. Metzker et al. Cleavage of a reversible terminator may be achieved by, for example, irradiating a nucleic acid molecule including the reversible terminator. In some instances the plurality of nucleotides may not comprise a terminated nucleotide.

One or more nucleotides of the plurality of nucleotides may be labeled with a dye, fluorophore, or quantum dot. For example, the solution may comprise labeled nucleotides. In another example, the solution may comprise unlabeled nucleotides. In another example, the solution may comprise a mixture of labeled and unlabeled nucleotides.

In a third operation 1630, the method may comprise subjecting the nucleic acid molecule to a primer extension reaction. The primer extension reaction may be conducted under conditions sufficient to incorporate at least one nucleotide from the plurality of nucleotides into a growing strand that is complementary to the nucleic acid molecule. The nucleotide incorporated may or may not be labeled. In some cases, the operation 1630 may further comprise modifying at least one nucleotide. Modifying the nucleotide may comprise labeling the nucleotide. For instance, the nucleotide may be labeled, such as with a dye, fluorophore, or quantum dot. The nucleotide may be cleavably labeled. In some instances, modifying the nucleotide may comprise activating (e.g., stimulating) a label of the nucleotide.

In a fourth operation 1640, the method may comprise detecting a signal indicative of incorporation of the at least one nucleotide. The signal may be an optical signal. The signal may be a fluorescence signal. The signal may be detected during motion of the substrate. The signal may be detected following termination of the motion. The signal may be detected while the nucleic acid molecule to be sequenced is in fluid contact with the solution. The signal may be detected following fluid contact of the nucleic acid molecule with the solution. The operation 1640 may further comprise modifying a label of the at least one nucleotide. For instance, the operation 1640 may further comprise cleaving the label of the nucleotide (e.g., after detection). The nucleotide may be cleaved by one or more stimuli, such as exposure to a chemical, an enzyme, light (e.g., ultraviolet light), or heat. Once the label is cleaved, a signal indicative of the incorporated nucleotide may not be detectable with one or more detectors.

The method 1600 may further comprise repeating operations 1620, 1630, and/or 1640 one or more times to identify one or more additional signals indicative of incorporation of one or more additional nucleotides, thereby sequencing the nucleic acid molecule. The method 1600 may comprise repeating operations 1620, 1630, and/or 1640 one or more times in an iterative manner. For each iteration, an additional signal may indicate incorporation of an additional nucleotide. The additional nucleotide may be the same nucleotide as detected in the previous iteration. The additional nucleotide may be a different nucleotide from the nucleotide detected in the previous iteration. In some instances, at least one nucleotide may be modified (e.g., labeled and/or cleaved) between each iteration of the operations 1620, 1630, and/or 1640. For instance, the method may comprise repeating the operations 1620, 1630, and/or 1640 at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, or 1,000,000,000 times. The method may comprise repeating the operations 1620, 1630, and/or 1640 a number of times that is within a range defined by any two of the preceding values. The method 1600 may thus result in the sequencing of a nucleic acid molecule of any size.

The method may comprise directing different solutions to the array during motion of the substrate. For instance, the method may comprise directing a first solution containing a first type of nucleotide (e.g., in a plurality of nucleotides of the first type) to the array, followed by a second solution containing a second type of nucleotide, followed by a third type of nucleotide, followed by a fourth type of nucleotide, etc. In another example, different solutions may comprise different combinations of types of nucleotides. For example, a first solution may comprise a first canonical type of nucleotide (e.g., A), a second solution may comprise a second canonical type of nucleotide (e.g., C), a third solution may comprise a third canonical type of nucleotide (e.g., T), and a fourth solution may comprise a fourth canonical type (e.g., G) of nucleotide. In another example, a first solution may comprise a first canonical type of nucleotide (e.g., A) and a second canonical type of nucleotide (e.g., C), and a second solution may comprise the first canonical type of nucleotide (e.g., A) and a third canonical type of nucleotide (e.g., T), and a third solution may comprise the first canonical type, second canonical type, third canonical type, and a fourth canonical type (e.g., G) of nucleotide. In another example, a first solution may comprise a mixture of labeled and unlabeled nucleotides, and a second solution may comprise unlabeled nucleotides. In another example, a first solution may comprise labeled nucleotides, and a second solution may comprise unlabeled nucleotides, and a third solution may comprise a mixture of labeled and unlabeled nucleotides. In another example, a first solution may comprise a mixture of labeled and unlabeled nucleotides of a first canonical base type (e.g., A), a second solution may comprise unlabeled nucleotides of the first canonical base type (e.g., A), a third solution may comprise a mixture of labeled and unlabeled nucleotides of a second canonical base type (e.g., C), a fourth solution may comprise unlabeled nucleotides of the second canonical base type (e.g., C), a fifth solution may comprise a mixture of labeled and unlabeled nucleotides of a third canonical base type (e.g., T), a sixth solution may comprise unlabeled nucleotides of the third canonical base type (e.g., T), a seventh solution may comprise a mixture of labeled and unlabeled nucleotides of a fourth canonical base type (e.g., G), and an eighth solution may comprise unlabeled nucleotides of the fourth canonical base type (e.g., G). The method may comprise directing at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, or 1,000,000,000 types of solutions to the array. The method may comprise directing a number of types of solutions that is within a range defined by any two of the preceding values to the array.

Many variations, alterations, and adaptations based on the method 1600 provided herein are possible. For example, the order of the operations of the method 1600 may be changed, some of the operations removed, some of the operations duplicated, and additional operations added as appropriate. Some of the operations may be performed in succession. Some of the operations may be performed in parallel. Some of the operations may be performed once. Some of the operations may be performed more than once. Some of the operations may comprise sub-operations. Some of the operations may be automated. Some of the operations may be manual. Some of the operations may be performed separately, e.g., in different locations or during different steps and/or processes. For example, directing a solution comprising a plurality of probes to the substrate may occur separately from the reaction and detection processes.

For example, in some cases, in the third operation 1630, instead of facilitating a primer extension reaction, the nucleic acid molecule may be subject to conditions to allow transient binding of a nucleotide from the plurality of nucleotides to the nucleic acid molecule. The transiently bound nucleotide may be labeled. The transiently bound nucleotide may be removed, such as after detection (e.g., see operation 1640). Then, a second solution may be directed to the substrate, this time under conditions to facilitate the primer extension reaction, such that a nucleotide of the second solution is incorporated (e.g., into a growing strand hybridized to the nucleic acid molecule). The incorporated nucleotide may be unlabeled. After washing, and without detecting, another solution of labeled nucleotides may be directed to the substrate, such as for another cycle of transient binding.

In some instances, such as for performing sequencing by ligation, the solution may comprise different probes. For example, the solution may comprise a plurality of oligonucleotide molecules. For example, the oligonucleotide molecules may have a length of about 2, 3, 4, 5, 6, 7, 8, 9, 10 bases or more. The oligonucleotide molecules may be labeled with a dye (e.g., fluorescent dye), as described elsewhere herein. In some instances, such as for detecting repeated sequences in nucleic acid molecules, such as homopolymer repeated sequences, dinucleotide repeated sequences, and trinucleotide repeated sequences, the solution may comprise targeted probes (e.g., homopolymer probe) configured to bind to the repeated sequences. The solution may comprise one type of probe (e.g., nucleotides). The solution may comprise different types of probes (e.g., nucleotides, oligonucleotide molecules, etc.). The solution may comprise different types of probes (e.g., oligonucleotide molecules, antibodies, etc.) for interacting with different types of analytes (e.g., nucleic acid molecules, proteins, etc.). Different solutions comprising different types of probes may be directed to the substrate any number of times, with or without detection between consecutive cycles (e.g., detection may be performed between some consecutive cycles, but not between some others), to sequence or otherwise process the nucleic acid molecule, depending on the type of processing.

### EXAMPLES

### Example 1: Reducing Aggregate Formation with Single-strand Binding Proteins

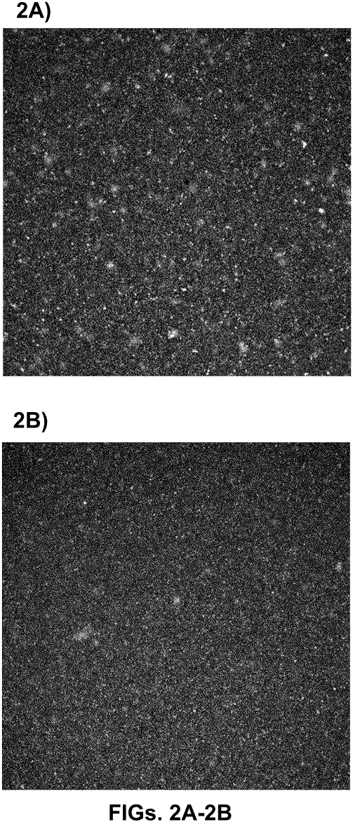

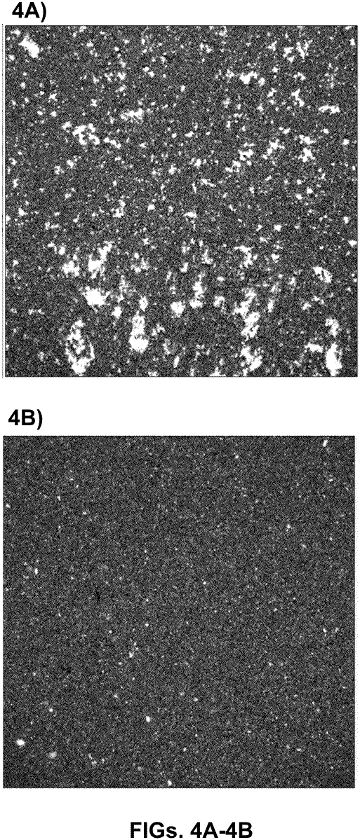

As described herein, single-stranded binding moieties such as single-strand binding proteins (SSBs) are known to bind tightly to single-stranded nucleic acid molecules (e.g., ssDNA) and may actually stimulate DNA polymerase activity. Various SSBs were evaluated for their impact on bead aggregation and sequencing quality. FIGS. 2A and 4A show images of a substrate loaded with particles comprising L225 amplified human genomic (HG) DNA following titration thereon in the absence of a single-strand binding protein. FIG. 2B shows an image of a substrate loaded with particles comprising L225 amplified HG DNA following titration thereon in the presence of 100 ng per million particles of a T4-derived single-strand binding protein. Similarly, FIG. 4B shows an image taken of a substrate loaded with particles following titration thereon in the presence of 400 ng per million beads of an E. coli-derived SSB protein. The substrates imaged in FIGS. 2A and 2B are loaded with 8 M/µL particles in TET buffer (Tris 10 mM, EDTA 0.1 mM and Triton X-100 0.01%). Tables 1 and 2 below summarizes differences observed between particles processed with and without SSB proteins. Table 3 below summarizes differences observed between particles processed with and without E.coli-derived SSB proteins.

TABLE 1

Observation of aggregates with and without SSB proteins.

| | Number of “good” (e.g., non-aggregated) Particles (~0.8 to 4 µm²) | Small Aggregates (~4 to 10 µm²) | Large Aggregates (over 10 µm²) | Average Intensity | Average Peak Intensity |
|---|---|---|---|---|---|
| 100 ng/million particles of T4-derived SSB protein | 25111 | 562 | 89 | 4196 | 246 |
| Without SSB protein | 11508 | 745 | 256 | 9537 | 311 |

TABLE 2

Sequencing quality with and without SSB proteins.

| | Number of Loaded Particles | Number of “Live” Particles | Error (70%) | F95 Genome | Coverage Genome | Passed RSQ filter? | Passed U-alignment? |
|---|---|---|---|---|---|---|---|
| Sample 1-with SSB protein | 1.7 billion | 1.1 billion | 0.08 | 2.1 | 63X | 82.2% | 63.9% |
| Sample 1- | 3.2 billion | 1.4 billion | 0.11 | 2.5 | 40X | 66.7% | 42.7% |
| without SSB protein | | | | | | | |
| Sample 2-with SSB protein | 1 billion | 0.7 billion | 0.08 | 2.5 | 34X | 82.1% | 67.9% |
| Sample 2-without SSB protein | 2 billion | 0.6 billion | 0.1 | 4.3 | 13X | 41.2% | 30.2% |

TABLE 3

Sequencing quality with and without E.coli-derived SSB proteins.

| | Number of Loaded Particles | Number of “Live” Particles | Error (70%) | F95 Genome | Coverage Genome |
|---|---|---|---|---|---|
| With SSB protein | 3 billion | 1.61 billion | 0.04 | 2.06 | 64X |
| Without SSB protein | 8.75 billion | 2.1 billion | 0.04 | 2.93 | 85X |

In Tables 2 and 3, ‘loaded particles’ refer to particles added to a substrate (e.g., wafer) while “‘live” particles’ refers to particles that adhered to a substrate and produced sequencing data. Error (70%) is the error associated with the top 70% of data (e.g., removing the bottom 30%). The F95 value is the fold increase in coverage needed to obtain coverage of 95% of the genome.

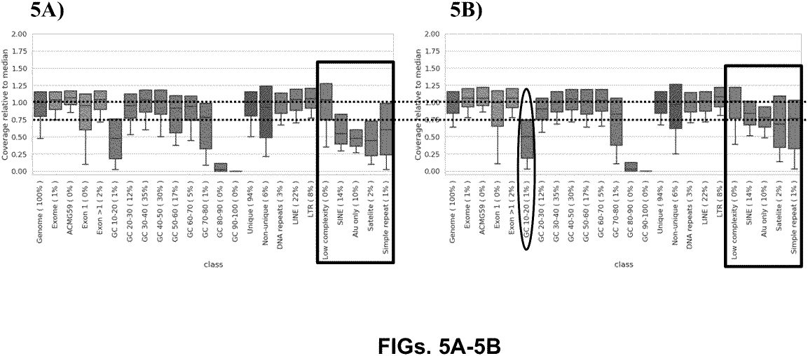

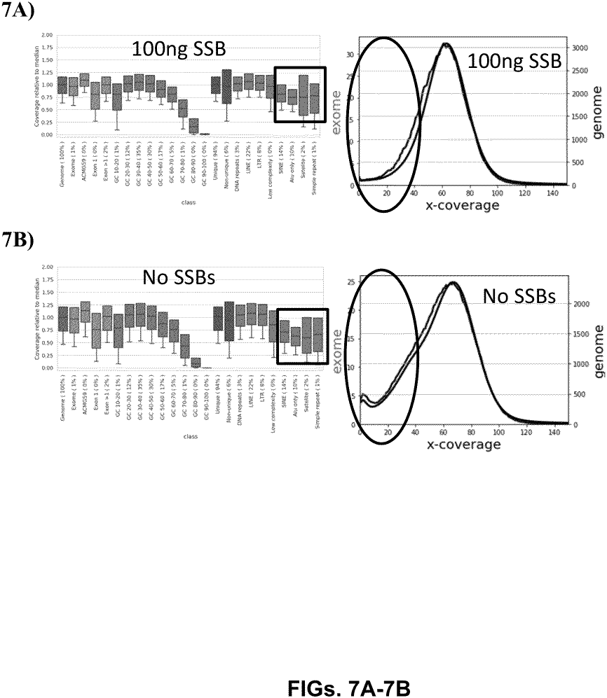

FIG. 5A shows relative sequencing coverage measured without E. coli-derived SSB protein while FIG. 5B shows relative sequencing coverage measured with 200 ng/million particles E. coli-derived SSB protein. As shown in the right-most portion of each figure, relative coverage was improved when SSB protein was used, particularly for repetitive elements that tend to self-aggregate. Similarly, FIG. 7A shows relative sequencing coverage measured with 100 ng E. coli-derived SSB protein/million B660 particles, while FIG. 7B shows relative sequencing coverage in the absence of any SSB protein. Table 4 below shows genome and exome coverage metrics corresponding to FIGS. 7A and 7B.

TABLE 4

Sequencing coverage measured with and without SSBs.

| | 100 ng SSB/million particles | | No SSBs | |
|---|---|---|---|---|
| | Genome | Exome | Genome | Exome |
| Mean coverage | 63.30 | 59.72 | 60.84 | 58.03 |
| Median coverage | 63.00 | 60.00 | 62.00 | 60.00 |
| % ≥ 20x | 97.93 | 97.75 | 94.32 | 93.40 |
| % duplicated | 29.87 | 29.87 | 28.27 | 28.27 |
| Input reads | 42503396.00 | 42503396.00 | 48210629.00 | 48210629.00 |
| F95 | 2.03 | 2.07 | 3.44 | 4.00 |
| F90 | 1.58 | 1.62 | 2.21 | 2.31 |
| F80 | 1.29 | 1.33 | 1.51 | 1.54 |

As shown in the figures and tables described above, the use of an SSB protein can significantly reduce the number of aggregates (e.g., clumps) in the particle population. Samples sequenced with SSBs also have increased levels of high-quality data, resulting in less filtering and increased coverage. These results suggest that SSB proteins may reduce secondary structure of single-stranded nucleic acid molecules coupled to particles and thus improve the ability of a polymerase to sequence through challenging sequences. Further, loading of particles (e.g., onto a substrate (wafer) for sequencing) is driven in part by charge. Addition of SSB proteins may thus be expected to decrease the disorder of single-stranded nucleic acid molecules to generate a more compacted particle with higher charge density, which may increase loading efficiency via a mechanism distinct from reducing bead aggregation. The loading experiments summarized in Table 2 are consistent with this hypothesis, as observed loading efficiency was higher for particles loaded in the presence of SSB proteins.

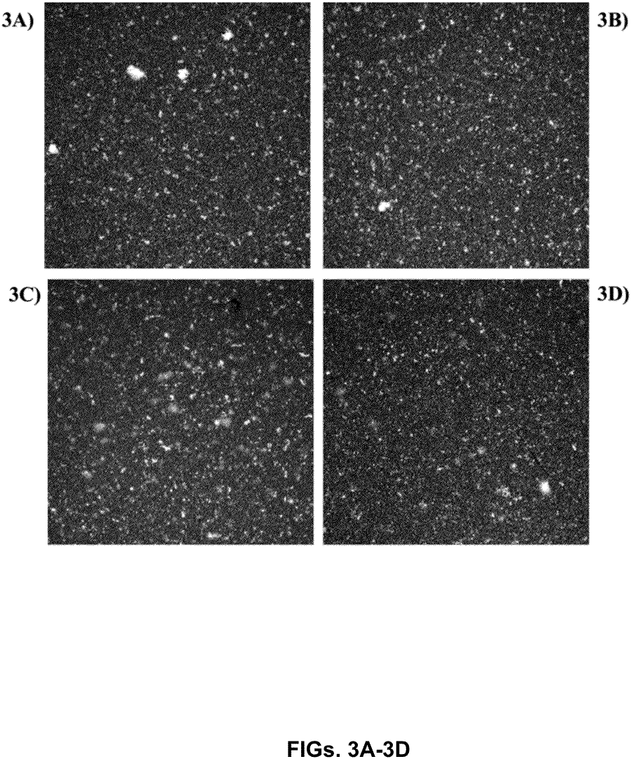

However, as shown in FIGS. 3A-3D, some SSB proteins may perform better than others. Substrates were loaded with 8 M/µL particles comprising L225 amplified HG DNA in TET buffer (Tris 10 mM, EDTA 0.1 mM and Triton X-100 0.01%) and incubated for about 30 minutes at room temperature. Following titration, samples were washed with TET. FIG. 3A shows an image of such a loaded substrate in the absence of an SSB protein while FIGS. 3B-3D show images of loaded substrates in the presence of 40 ng/million particles, 60 ng/million particles, or 80 ng/million particles, respectively, of Extreme Thermostable SSB protein (ET SSB). ET SSB may be added during PA26FAM hybridization and excess ET SSB may be washed away using, e.g., one or more TET washes. As shown in FIGS. 3A-3D, under the conditions tested, ET SSB protein did not substantially reduce particle aggregation. However, optimization of temperature, salt concentration, and SSB concentration, among other conditions, may facilitate reduction of particle aggregation with this SSB. ***

### Example 2: Reducing Aggregate Formation With Single-strand Binding Proteins and Randomers

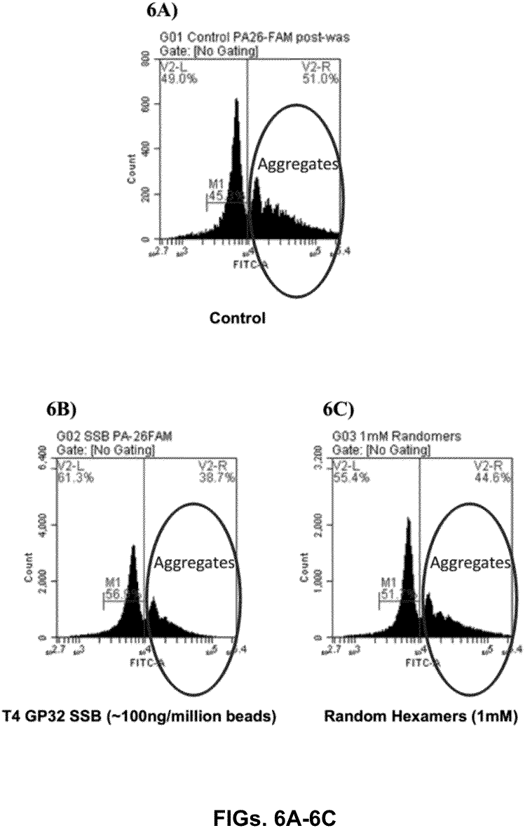

Both SSB proteins and randomers (e.g., nucleic acid molecules comprising random sequences, such as random hexamers) may be useful as single-stranded binding moieties. As shown in FIGS. 6A-6C, particle aggregates measured using flow cytometry were reduced using both about 100 ng/million particles of T4 GP32-derived SSB protein (FIG. 6B) and about 1 millimolar (mM) random hexamers (FIG. 6C) relative to a control (FIG. 6A). The mean and coefficient of variation for the respective samples are summarized in Table 5 below.

TABLE 5

Flow cytometry signals for particles treated with SSB proteins and randomers

| Sample | Mean signal | CV signal |
|---|---|---|
| Control | 68590.86 | 404.21% |
| SSB | 25313.75 | 138.03% |
| Randomers | 51172.15 | 260.66% |

### Example 4: Reducing Aggregate Formation With Oligomers

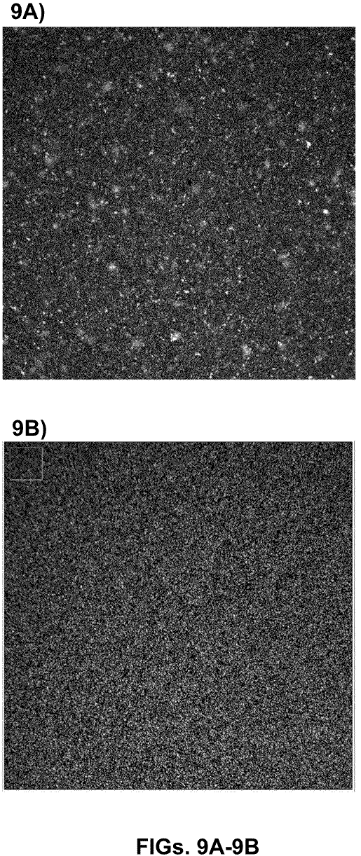

As described herein, oligomers including blocking primers may be used to block single-stranded portions of nucleic acid molecules coupled to beads to reduce particle aggregation. FIGS. 8A and 8B schematically illustrate the use of blocking primers to block single-stranded portions of nucleic acid molecules coupled to beads to provide double-stranded nucleic acid regions. FIG. 9A shows an image of a substrate loaded with particles comprising single-stranded nucleic acid molecules, while FIG. 9B shows an image of a substrate loaded with particles comprising single-stranded nucleic acid molecules treated with blocking oligomers to provide double-stranded nucleic acid molecules. As is evident in the figures, the use of blocking oligomers reduces particle aggregation, as indicated by the relatively uniformity of illuminated spots as seen in FIG. 9B compared to the larger bright spots that are seen in FIG. 9A.

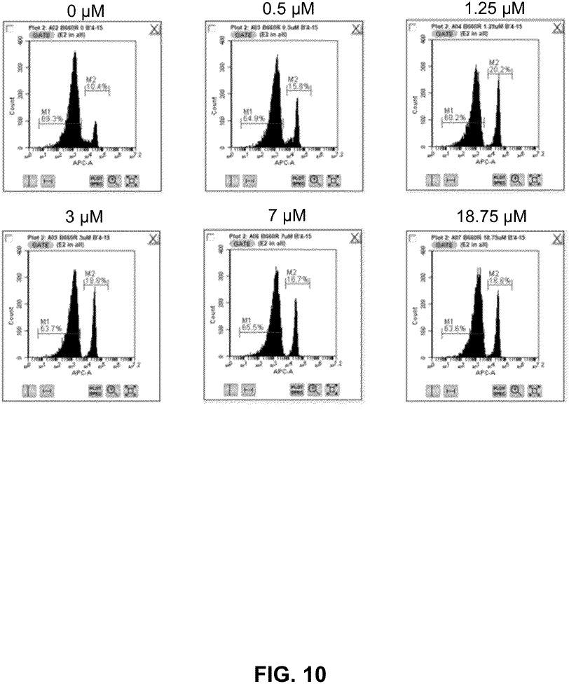

FIG. 10 includes histograms summarizing flow cytometric analysis of particles comprising single-stranded nucleic acid molecules treated with varying concentrations of blocking oligomers. Particle counts are plotted against intensity in the APC (a red fluorescing dye) channel. M1 values reference unamplified beads while M2 values reference amplified beads. Table 6 summarizes the M1 and M2 values for the respective histograms.

TABLE 6

Flow cytometric parameters.

| Concentration of blocking oligomer | M1 | M2 |
|---|---|---|
| 0 µM | 69.3% | 10.4% |
| 0.5 µM | 64.9% | 15.8% |
| 1.25 µM | 60.2% | 20.2% |
| 3 µM | 63.7% | 18.8% |
| 7 µM | 65.5% | 16.7% |
| 18.75 µM | 63.6% | 18.6% |

As shown in FIG. 10 and Table 6, the addition of blocking oligomers increases signal from amplified beads, demonstrating that the use of blocking oligomers can effectively reduce particle aggregation.

### Example 5: Prolonged Exposure to Mg²⁺ Encourages Bead Aggregation

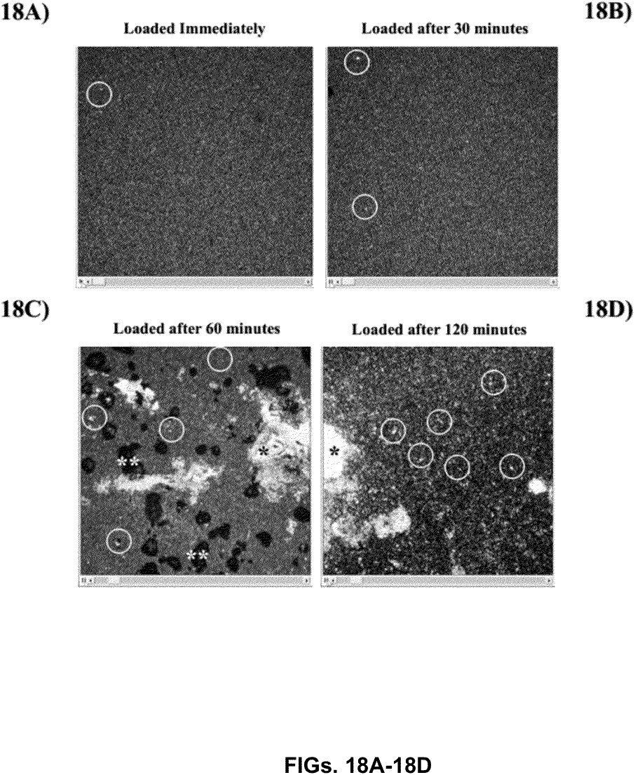

As described herein, prolonged exposure of a plurality of beads to cations, such as divalent cations Mg²⁺ or Ca²⁺, encourages bead aggregation. To identify a role of cations in the bead aggregation, beads (ThermoFisher’s Ion sphere particles (ISPs)) were prepared by attaching DNAs amplified by emulsion PCR (emPCR) were incubated with a TTM buffer (10 mM of Tris, pH= 7.0; 0.055% Tergitol; 10 mM MgCl₂) for various amount of times (0 minute, 30 minutes, 60 minutes, and 120 minutes) in a sample tube before dispensing the beads onto wafers. As shown in FIGS. 18A-18D, which show coupon wafers (e.g., sections of wafers) of loaded beads, loading the beads to the wafer immediately after incubation with the TTM buffer did not cause significant bead aggregation (e.g., in FIG. 18A). However, the numbers of bead aggregates on the wafer increased with the time the beads were incubated in the TTM buffer, as indicated by the bright white spots highlighted by circles, i.e., with longer exposure to the Mg²⁺. Therefore, FIG. 18B illustrates the result of bead loading after 30 minutes of incubation of the beads with TTM buffer. FIG. 18C illustrates the result of bead loading after 60 minutes of incubation of the beads with TTM buffer. FIG. 18D illustrate the result of bead loading after 120 minutes of the beads with TTM buffer. FIG. 18B shows slightly increased bead aggregation as compared with FIG. 18A. FIGS. 18C and 18D both show significantly increased bead aggregation as compared with FIG. 18A. Therefore, this example shows that prolonged exposure to cations (e.g., in solution) encourages bead aggregation. One potential method to reduce bead aggregation is to minimize the exposure of the beads to cations (e.g., Mg²⁺ in TTM buffer) before dispensing the beads onto the substrate. (e.g., as described in Example 6).

### Example 6: Incubating the Substrate, Instead of the Beads, With Mg²⁺ Promotes High Occupancy of Beads Without Aggregation

As described herein, incubating a substrate with cations, such as divalent cations including Mg²⁺ or Ca²⁺, encourages high occupancy of the substrate with beads and minimizes bead aggregation. Although cations promote bead aggregation when the beads are exposed to the ions, for prolonged periods (e.g., as during incubation with a cation-containing buffer as described in Example 5), the same cations also advantageously facilitate dense packing and immobilization of the beads onto small features (i.e., micrometer level features) of the substrate. Aggregation and dense packing are inherently related effects of loading beads onto a substrate. One goal with bead loading is to minimize aggregation, which degrades the sequencing information obtainable from a loaded substrate, while still permitting dense packing, which increases sequencing efficiency. The cations themselves can also screen the high, negative charges of the beads and reduce bead-bead repulsion that occurs on substrates with small feature sizes. It is therefore desirable to investigate the parameters (e.g., the concentration or the incubation time of the cations) of the cations for the treatment, either separately or together, of the substrate and the beads.

To investigate the effectiveness of cations in promoting surface occupancy while still minimizing bead aggregation, wafers with a pitch size of 1.8 µm were incubated (prewet) with a TT (10 mM of Tris, pH= 7.0; 0.055% Tergitol) buffer containing various amounts of Mg²⁺ (50 mM, 100 mM, 200 mM, and 300 mM) for 0.5-1 minute. The beads were incubated with the TT buffer that lacked Mg²⁺ for 60 minutes in a sample tube prior to being dispensed onto the wafer. As a control, both the wafer and beads were incubated with a TTM buffer (10 mM of Tris, pH= 7.0; 0.055% Tergitol; 10 mM MgCl₂) for 0.5-1 and 60 minutes, respectively. Table 7 summarizes the resulting average substrate occupancy percentage on the wafers with the beads in different experiments.

TABLE 7

Coupon wafer occupancy with beads on a coupon wafer with a pitch size of 1.8 µm

| Coupon wafer incubation buffer | Bead incubation buffer | Average substrate occupancy (%) |
|---|---|---|
| TTM buffer | TTM buffer | 94 |
| TT buffer + 50 mM Mg²⁺ | TT buffer | 91 |
| TT buffer + 100 mM Mg²⁺ | TT buffer | 92 |
| TT buffer + 200 mM Mg²⁺ | TT buffer | 92 |
| TT buffer + 300 mM Mg²⁺ | TT buffer | 94 |

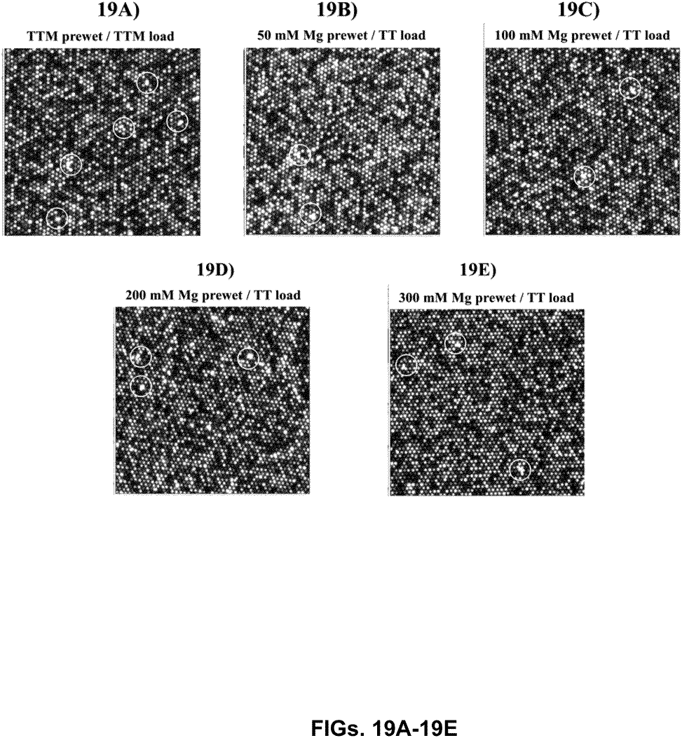

Substrate occupancy measures how much area or how many locations (i.e., micrometer level features) of the substrate are covered with beads. It does not inform on bead aggregation, but rather success of bead loading. As shown in FIGS. 19A-19E, high average substrate occupancies were achieved in the wafers incubated with various concentrations of Mg²⁺. FIG. 19B show the beads incubated with 50 mM Mg²; FIG. 19C show the beads incubated with 100 mM Mg²; FIG. 19D show the beads incubated with 200 mM Mg²; FIG. 19E show the beads incubated with 300 mM Mg². The beads incubated in buffer without any Mg²⁺ (i.e., when the beads were incubated with the TT buffer) showed significantly less bead aggregation on wafer as compared to the control experiment (FIG. 19A) where the beads and the wafers were both incubated in buffer containing Mg²⁺ (i.e., TTM buffer).

Even when the beads were exposed to the same total amount of Mg²⁺ (e.g., through either incubation of the beads in buffer containing Mg²⁺ or from prewetting a wafer in buffer containing Mg²⁺), incubation in Mg²⁺ containing buffer resulted in aggregates while Mg²⁺ prewetting discouraged bead aggregation without affecting the substrate occupancy. Thus, the timing (i.e., on or off wafer) of bead exposure to Mg²⁺ is surprisingly important. Table 8 summarizes average substrate occupancy when beads were incubated with or without buffer containing Mg²⁺ (e.g., the TT buffer or the TTM buffer, respectively), while the wafer was incubated with TT buffer containing Mg²⁺ (at either 50 mM or 100 mM) for 0 min. The wafers in this experiment had an average pitch size of 1.5 µm.

TABLE 8

Coupon wafer occupancy with beads on a coupon wafer with a pitch size of 1.5 µm

| Coupon wafer incubation buffer | Bead incubation buffer | Average substrate occupancy (%) |
|---|---|---|
| TT buffer + 50 mM Mg²⁺ | TT buffer + 50 mM Mg²⁺ | 88 |
| TT buffer + 100 mM Mg²⁺ | TT buffer | 88 |

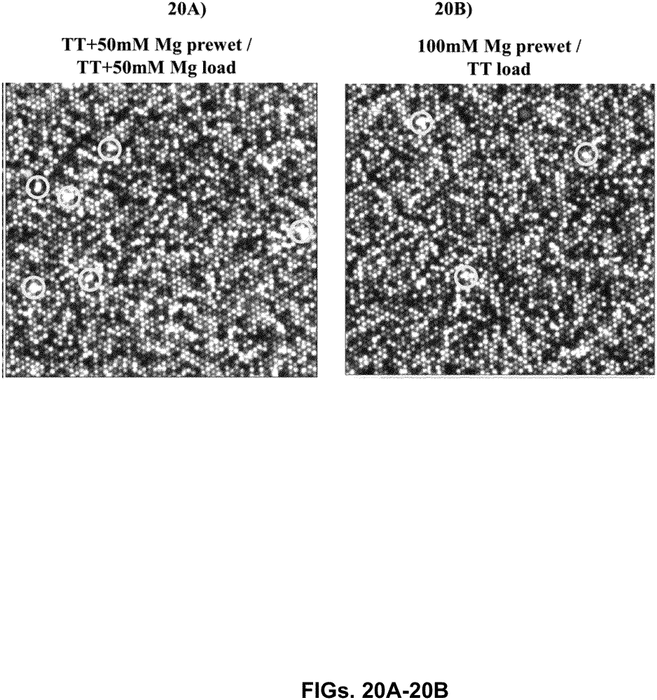

A high occupancy of beads on the substrates was achieved in either scenario. In addition, as shown in FIGS. 20A and 20B, the beads incubated without any Mg²⁺ (i.e., when the beads were incubated with the TT buffer) showed significantly less bead aggregation compared to that of the control, even though the beads were exposed to the same amounts of Mg²⁺ in total in both experiments.

Therefore, it can be concluded that exposure of beads themselves to cations encourages bead aggregation, but that incubation (prewetting) of the substrate with the same cations promotes substrate occupancy without promoting the bead aggregation. Hence, one way to reduce the bead aggregation effect is to minimize the exposure of beads to cations before dispensing onto a substrate. The benefits of dense loading of beads onto wafers can be achieved by incubating the wafers (i.e., alone without the beads, prior to loading) with cations.

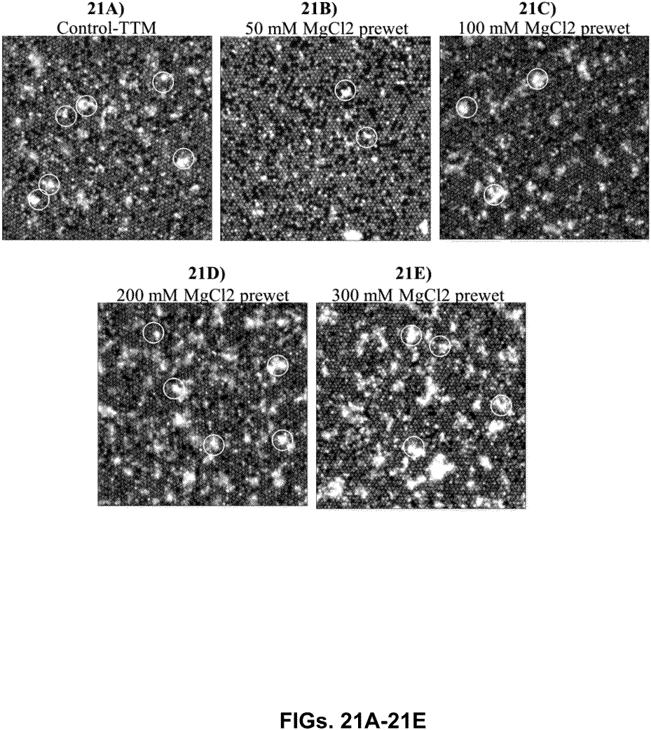

To test whether the same strategy to reduce the bead aggregation while promoting the substrate occupancy would work for other types of beads and substrate layouts, amplified IH beads (B1434 (10%-tBA ATRP in THF)) were prepared by coating the beads with polymer + oligonucleotide conjugate. The beads were attached with DNA amplified by emPCR and fluorescently labeled by hybridizing the amplified DNA strands with a dye-conjugated complementary oligonucleotide probe sequence (i.e., PA39FAM). The labeled beads were incubated in TT buffer (prewet) before loading onto a substrate with a 1.5 µm pitch size. The beads were incubated in 20 µL TT buffer with a concentration of about 20,000,000 beads per µL. The substrate was incubated with the TT buffer with various amounts of Mg²⁺ (e.g., 50 mM (FIG. 21B), 100 mM (FIG. 21C), 200 mM (FIG. 21D), and 300 mM (FIG. 21E)) for 0.5-1 min. As a control (FIG. 21A), the wafer and the beads were both incubated with the TTM buffer for 0.5-1 min and 60 min, respectively. As shown in FIGS. 21A-21E, in all cases when the beads were incubated with the TT buffer, while the wafers were prewet with buffers containing Mg²⁺, high substrate occupancies were observed. The wafer incubated with 50 mM Mg²⁺ showed the least bead aggregation. In contrast, the wafers incubated with either 200 mM or 300 mM Mg²⁺ showed high levels of bead aggregation. Therefore, excess amounts of Mg²⁺ are capable of leading to bead aggregation even when used purely for prewetting wafers.

These experiments demonstrate that Ca²⁺ worked as well as Mg²⁺ in promoting substrate occupancy. However, using Zn²⁺, another divalent cation, significantly increased aggregation of the beads: In one example, the wafers and the beads were incubated (prewet) with a TT (10 mM of Tris, pH= 7.0; 0.055% Tergitol) buffer containing 10 mM Zn²⁺ (provided in the form of zinc acetate) for 0.5-1 minute and 60 minutes, respectively. After dispensing the beads on the wafers, irreversible and massive bead aggregation was observed by the naked eye. Individual beads could not be resolved under the microscope; only aggregates were observed. In a second example, the wafers were incubated (prewet) with a TT (10 mM of Tris, pH= 7.0; 0.055% Tergitol) buffer containing 10 mM Zn²⁺ (provided in the form of zinc acetate) for 0.5-1 minute. The beads were incubated with the TT buffer that lacked Zn²⁺ for 60 minutes in a sample tube prior to being dispensed onto the wafer. Low level of bead aggregation was observed. However, the bead loading was sparse and not dense across coupon surface. Hence, some, but not all divalent cations, can promote the substrate occupancy without inducing bead aggregation.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EMBODIMENTS

1. A method for processing a plurality of particles, comprising:
(a) providing a first particle of said plurality of particles comprising a first nucleic acid molecule immobilized thereto, wherein said first nucleic acid molecule comprises a first single-stranded portion;
(b) contacting said first nucleic acid molecule with a first single-stranded binding moiety under conditions sufficient to couple said first single-stranded binding moiety to said first single-stranded portion of said first nucleic acid molecule to yield a first treated particle comprising a first blocked nucleic acid molecule immobilized thereto, wherein said first blocked nucleic acid molecule comprises said first nucleic acid molecule coupled to said first single-stranded binding moiety; and
(c) providing said first treated particle in a solution comprising a second treated particle, wherein said second treated particle comprises a second nucleic acid molecule comprising a second single-stranded portion coupled to a second single-stranded binding moiety.

2. The method of embodiments 1, wherein said plurality of particles is a plurality of beads.

3. The method of embodiments 1 or 2, wherein said first nucleic acid molecule comprises deoxyribonucleic acid (DNA) nucleotides, ribonucleic acid (RNA) nucleotides, or a combination thereof.

4. The method of any one of embodiments 1-3, wherein said first single-stranded portion comprises single-stranded deoxyribonucleic acid (ssDNA), ribonucleic acid (RNA), or a combination thereof.

5. The method of any one of embodiments 1-4, wherein said first nucleic acid molecule comprises a sequence of a sample nucleic acid molecule, or a complement thereof.

6. The method of any one of embodiments 1-4, wherein said first nucleic acid molecule comprises a priming sequence, or a complement thereof.

7. The method of embodiment 6, wherein said priming sequence is a targeted priming sequence.

8. The method of embodiment 6, wherein said priming sequence comprises a random N-mer sequence.

9. The method of any one of embodiments 1-8, wherein said first nucleic acid molecule comprises a barcode sequence or a unique molecular identifier sequence.

10. The method of any one of embodiments 1-9, wherein (b) comprises providing a reaction mixture comprising said first single-stranded binding moiety.

11. The method of embodiment 10, wherein said reaction mixture comprises a salt.

12. The method of embodiment 10 or 11, wherein said reaction mixture comprises spermine.

13. The method of any one of embodiments 10-12, wherein said reaction mixture comprises cobalt hexammine.

14. The method of any one of embodiments 1-13, wherein said first single-stranded binding moiety comprises a single-stranded binding (SSB) protein.

15. The method of embodiment 14, wherein said SSB protein is a T4 phage-derived SSB protein, an Escherichia coli-derived SSB protein, or an Extreme Thermostable SSB protein.

16. The method of any one of embodiments 1-13, wherein said first single-stranded binding moiety comprises a third nucleic acid molecule.

17. The method of embodiment 16, wherein said third nucleic acid molecule comprises a random N-mer.

18. The method of embodiment 17, wherein N is between 6 and 12.

19. The method of embodiment 18, wherein said second nucleic acid molecule comprises 6 bases.

20. The method of embodiment 16, wherein said third nucleic acid molecule has sequence complementarity to a sequence of said first single-stranded portion of said first nucleic acid molecule.

21. The method of any one of embodiments 16-20, wherein said first single-stranded binding moiety comprises a loop structure.

22. The method of embodiment 21, wherein said loop structure is included in a hairpin moiety.

23. The method of embodiment 21, wherein said first single-stranded binding moiety comprises a single-stranded nucleic acid molecule coupled to a moiety comprising said loop structure.

24. The method of any one of embodiments 1-23, wherein said first particle comprises a first plurality of nucleic acid molecules immobilized thereto, wherein said first plurality of nucleic acid molecules comprises said nucleic acid molecule, and wherein said first plurality of nucleic acid molecules has at least partial sequence identity to a first nucleic acid sequence.

25. The method of embodiment 24, wherein said second treated particle comprises a second plurality of nucleic acid molecules immobilized thereto, wherein said second plurality of nucleic acid molecules is different from said first plurality of nucleic acid molecules, and wherein said second plurality of nucleic acid molecules has at least partial sequence identity to a second nucleic acid sequence.

26. The method of embodiment 25, wherein said second nucleic acid sequence is different than said first nucleic acid sequence.

27. The method of embodiment 25, wherein said second nucleic acid sequence and said first nucleic acid sequence are identical.

28. The method of any one of embodiments 24-27, wherein said first plurality of nucleic acid molecules comprises at least 1,000 nucleic acid molecules.

29. The method of embodiment 28, wherein said first plurality of nucleic acid molecules comprises at least 100,000 nucleic acid molecules.

30. The method of any one of embodiments 1-29, wherein said plurality of particles comprises at least 10,000,000 particles.

31. The method of embodiment 30, wherein said plurality of particles comprises at least 1,000,000,000 particles.

32. The method of any one of embodiments 1-31, wherein said first single-stranded binding moiety and said second single-stranded binding moiety are of a same type.

33. The method of any one of embodiments 1-31, wherein said first single-stranded binding moiety and said second single-stranded binding moiety are of different types.

34. The method of any one of embodiments 1-33, further comprising immobilizing said first particle to a substrate.

35. The method of embodiment 34, wherein said first particle and said second particle are immobilized to different independently addressable locations of said substrate.

36. The method of embodiment 35, wherein said independently addressable locations are substantially planar.

37. The method of embodiment 35, wherein said independently addressable locations comprise one or more wells.

38. The method of embodiment 35, wherein said independently addressable locations comprise one or more pillars.

39. The method of any one of embodiments 34-38, wherein said substrate comprises said solution.

40. The method of any one of embodiments 1-32, wherein (b) comprises contacting said plurality of particles with a plurality of single-stranded binding moieties under conditions sufficient to couple said single-stranded binding moieties of said plurality of single-stranded binding moieties to single-stranded portions of nucleic acid molecules immobilized to particles of said plurality of particles to yield a plurality of treated particles comprising said first treated particle and said second treated particle.

41. The method of embodiment 40, wherein said plurality of single-stranded binding moieties comprises a single type of single-stranded binding moiety.

42. The method of embodiment 40, wherein said plurality of single-stranded binding moieties comprises a plurality of different types of single-stranded binding moieties.

43. The method of any one of embodiments 40-42, wherein, at a given time subsequent to (b), said plurality of treated particles comprises a number of particles that is at least 50% of the number of particles of said plurality of particles.

44. The method of embodiment 43, wherein, at a given time subsequent to (b), said plurality of treated particles comprises a number of particles that is at least 70% of the number of particles of said plurality of particles.

45. The method of embodiments 44, wherein, at a given time subsequent to (b), said plurality of treated particles comprises a number of particles that is at least 90% of the number of particles of said plurality of particles.

46. The method of any one of embodiments 40-45, wherein, subsequent to (b), particle aggregates, comprising two or more treated particles of said plurality of treated particles, having a dimension of at least about 1 micrometer (µm) are absent from said plurality of treated particles.

47. The method of any one of embodiments 40-46, wherein, subsequent to (b), no more than 1% of treated particles of said plurality of treated particles are included in a particle aggregate comprising two or more treated particles of said plurality of treated particles.

48. The method of any one of embodiments 40-47, further comprising immobilizing said plurality of treated particles to a substrate.

49. The method of embodiment 48, wherein said plurality of treated particles are immobilized to different independently addressable locations of said substrate.

50. The method of embodiment 49, wherein said independently addressable locations are substantially planar.

51. The method of embodiment 49, wherein said independently addressable locations comprise one or more wells.

52. The method of embodiment 49, wherein said independently addressable locations comprise one or more pillars.

53. The method of any one of embodiments 1-52, further comprising, prior to (a), denaturing a double-stranded portion of said first nucleic acid molecule to yield said first single-stranded portion.

54. The method of any one of embodiments 1-53, further comprising sequencing said first single-stranded portion of said first nucleic acid molecule, or a portion thereof.

55. A method for processing a plurality of particles, comprising:
a. providing said plurality of particles, wherein each particle of at least a subset of said plurality of particles comprises a nucleic acid molecule of a plurality of nucleic acid molecules immobilized thereto, wherein each nucleic acid molecule of at least said subset of said plurality of nucleic acid molecules comprises a single-stranded portion; and
b. contacting said plurality of particles with a plurality of single-stranded binding moieties under conditions sufficient for single-stranded binding moieties of said plurality of single-stranded binding moieties to couple to single-stranded portions of nucleic acid molecules of said at least said subset of said plurality of nucleic acid molecules, wherein, subsequent to (b), said plurality of particles are included in a solution, and wherein no more than 1% of particles of said plurality of particles are included in a particle aggregate comprising two or more particles of said plurality of particles.

56. The method of embodiment 55, wherein said plurality of particles is a plurality of beads.

57. The method of embodiment 55 or 56, wherein said plurality of nucleic acid molecules comprises a plurality of deoxyribonucleic acid (DNA) molecules, a plurality of ribonucleic acid (RNA) molecules, or a combination thereof.

58. The method of any one of embodiments 55-57, wherein said plurality of nucleic acid molecules comprise sequences of sample nucleic acid molecules, or complements thereof.

59. The method of any one of embodiments 55-58, wherein said plurality of nucleic acid molecules comprise a plurality of priming sequences, or complements thereof.

60. The method of embodiment 59, wherein said plurality of priming sequences comprises a plurality of targeted priming sequences.

61. The method of embodiment 59, wherein said plurality of priming sequences comprises a plurality of random N-mer sequences.

62. The method of any one of embodiments 55-61, wherein (b) comprises providing a reaction mixture comprising said plurality of single-stranded binding moieties.

63. The method of embodiment 62, wherein said reaction mixture comprises a salt.

64. The method of embodiment 62 or 63, wherein said reaction mixture comprises spermine.

65. The method of any one of embodiments 62-64, wherein said reaction mixture comprises cobalt hexammine.

66. The method of any one of embodiments 55-65, wherein said plurality of single-stranded binding moieties comprises a plurality of single-stranded binding (SSB) proteins.

67. The method of embodiment 66, wherein said plurality of single-stranded binding (SSB) proteins comprises T4 phage-derived SSB proteins, Escherichia coli-derived SSB proteins, Extreme Thermostable SSB proteins, or a combination thereof.

68. The method of any one of embodiments 55-67, wherein said plurality of single-stranded binding moieties comprises an additional plurality of nucleic acid molecules.

69. The method of embodiment 68, wherein said additional plurality of nucleic acid molecules comprises a plurality of random N-mers.

70. The method of embodiment 68, wherein said additional plurality of nucleic acid molecules has sequence complementarity to sequences of said single-stranded portions of said at least said subset of said plurality of nucleic acid molecules.

71. The method of any one of embodiments 68-70, wherein said additional plurality of nucleic acid molecules comprises a plurality of loop structures.

72. The method of embodiment 71, wherein said plurality of loop structures is included in a plurality of hairpin moieties.

73. The method of embodiment 71, wherein said additional plurality of nucleic acid molecules comprises a plurality of single-stranded nucleic acid molecules coupled to a plurality of moieties comprising said plurality of loop structures.

74. The method of any one of embodiments 55-73, wherein said plurality of single-stranded binding moieties comprises a single type of single-stranded binding moiety.

75. The method of any one of embodiments 55-73, wherein said plurality of single-stranded binding moieties comprises a plurality of different types of single-stranded binding moieties.

76. The method of any one of embodiments 55-75, wherein nucleic acid molecules of said plurality of nucleic acid molecules immobilized to a given particle of said at least said subset of said plurality of particles have sequence identity to a first nucleic acid sequence.

77. The method of embodiment 76, wherein additional nucleic acid molecules of said plurality of nucleic acid molecules immobilized to an additional given particle of said at least said subset of said plurality of particles have sequence identity to a second nucleic acid sequence.

78. The method of embodiment 77, wherein said first nucleic acid sequence and said second nucleic acid sequence are identical.

79. The method of embodiment 77, wherein said first nucleic acid sequence and said second nucleic acid sequence are different.

80. The method of any one of embodiments 55-79, wherein said plurality of particles comprises at least 10,000,000 particles.

81. The method of embodiment 80, wherein said plurality of particles comprises at least 1,000,000,000 particles.

82. The method of any one of embodiments 55-81, wherein, at a given time subsequent to (b), at least 50% of particles of said plurality of particles comprise a nucleic acid molecule of said plurality of nucleic acid molecules that is coupled to a single-stranded binding moiety of said plurality of single-stranded binding moieties.

83. The method of embodiment 82, wherein, at a given time subsequent to (b), at least 70% of particles of said plurality of particles comprise a nucleic acid molecule of said plurality of nucleic acid molecules that is coupled to a single-stranded binding moiety of said plurality of single-stranded binding moieties.

84. The method of embodiment 83, wherein, at a given time subsequent to (b), at least 90% of particles of said plurality of particles comprise a nucleic acid molecule of said plurality of nucleic acid molecules that is coupled to a single-stranded binding moiety of said plurality of single-stranded binding moieties.

85. The method of any one of embodiments 55-84, wherein, subsequent to (b), no more than 0.1% of particles of said plurality of particles are included in a particle aggregate comprising two or more particles of said plurality of particles.

86. The method of embodiments 55-85, wherein, subsequent to (b), no more than 0.01% of particles of said plurality of particles are included in a particle aggregate comprising two or more particles of said plurality of particles.

87. The method of embodiments 55-86, wherein, subsequent to (b), no more than 0.001% of particles of said plurality of particles are included in a particle aggregate comprising two or more particles of said plurality of particles.

88. The method of any one of embodiments 55-87, wherein particle aggregates, comprising two or more particles of said plurality of particles, having a dimension of at least about 5 micrometer (µm) are absent from said plurality of particles.

89. The method of embodiment 88, wherein particle aggregates, comprising two or more particles of said plurality of particles, having a dimension of at least about 1 micrometer (µm) are absent from said plurality of particles.

90. The method of any one of embodiments 55-89, further comprising immobilizing said plurality of particles to a substrate.

91. The method of embodiment 90, wherein said plurality of particles are immobilized to different independently addressable locations of said substrate.

92. The method of embodiment 91, wherein said independently addressable locations are substantially planar.

93. The method of embodiment 91, wherein said independently addressable locations comprise one or more wells.

94. The method of embodiment 91, wherein said independently addressable locations comprise one or more pillars.

95. The method of any one of embodiments 55-94, further comprising, prior to (a), denaturing double-stranded portions of said at least said subset of said plurality of nucleic acid molecules to yield said single-stranded portions.

96. The method of any one of embodiments 55-95, further comprising sequencing said single-stranded portions of said at least said subset of said plurality of nucleic acid molecules, or portions thereof.

97. A composition, comprising:
a suspension comprising:
(i) a plurality of particles comprising a first particle and a second particle, wherein said plurality of particles comprises a plurality of nucleic acid molecules immobilized thereto, wherein said first particle comprises a first nucleic acid molecule of said plurality of nucleic acid molecules immobilized thereto and said second particle comprises a second nucleic acid molecule of said plurality of nucleic acid molecules immobilized thereto, wherein said first nucleic acid molecule comprises a single-stranded portion; and (ii) a single-stranded binding moiety, wherein said single-stranded binding moiety is configured to couple to said single-stranded portion of said first nucleic acid molecule, wherein said first particle is in fluidic communication with said second particle.

98. The composition of embodiment 97, wherein said plurality of particles is a plurality of beads.

99. The composition of embodiment 97 or 98, wherein said plurality of nucleic acid molecules comprises a plurality of deoxyribonucleic acid (DNA) molecules, ribonucleic acid (RNA) molecules, or a combination thereof.

100. The composition of any one of embodiments 97-99, wherein said single-stranded portion of said first nucleic acid molecule comprises single-stranded deoxyribonucleic acid (ssDNA), ribonucleic acid (RNA), or a combination thereof.

101. The composition of any one of embodiments 97-100, wherein said first nucleic acid molecule comprises a sequence of a sample nucleic acid molecule, or a complement thereof.

102. The composition of any one of embodiments 97-101, wherein said first nucleic acid molecule comprises a priming sequence, or a complement thereof.

103. The composition of embodiment 102, wherein said priming sequence is a targeted priming sequence.

104. The composition of embodiment 102, wherein said priming sequence comprises a random N-mer sequence.

105. The composition of any one of embodiments 97-104, wherein said first nucleic acid molecule comprises a barcode sequence or a unique molecular identifier sequence.

106. The composition of any one of embodiments 97-105, further comprising one or more reagents for facilitating coupling of said single-stranded binding moiety and said single-stranded portion of said first nucleic acid molecule.

107. The composition of embodiment 106, wherein said one or more reagents comprises a salt.

108. The composition of embodiment 106 or 107, wherein said one or more reagents comprises spermine.

109. The composition of any one of embodiments 106-108, wherein said one or more reagents comprises cobalt hexammine.

110. The composition of any one of embodiments 97-109, wherein said single-stranded binding moiety comprises a single-stranded binding (SSB) protein.

111. The composition of embodiments 110, wherein said SSB protein is a T4 phage-derived SSB protein, an Escherichia coli-derived SSB protein, or an Extreme Thermostable SSB protein.

112. The composition of any one of embodiments 97-111, wherein said single-stranded binding moiety comprises a third nucleic acid molecule.

113. The composition of embodiment 112, wherein said third nucleic acid molecule comprises a random N-mer.

114. The composition of embodiment 113, wherein N is between 6 and 12.

115. The composition of embodiment 114, wherein said third nucleic acid molecule comprises 6 bases.

116. The composition of embodiment 112, wherein said third nucleic acid molecule has sequence complementarity to a sequence of said single-stranded portion of said first nucleic acid molecule.

117. The composition of any one of embodiments 113-116, wherein said single-stranded binding moiety comprises a loop structure.

118. The composition of embodiment 117, wherein said loop structure is included in a hairpin moiety.

119. The composition of embodiment 117, wherein said single-stranded binding moiety comprises a single-stranded nucleic acid molecule coupled to a moiety comprising said loop structure.

120. The composition of any one of embodiments 97-119, wherein said composition comprises a plurality of single-stranded binding moieties comprising said single-stranded binding moiety.

121. The composition of embodiment 120, wherein said plurality of single-stranded binding moieties are of a same type.

122. The composition of embodiment 120, wherein said plurality of single-stranded binding moieties comprises multiple types of single-stranded binding moieties.

123. The composition of any one of embodiments 120-122, wherein at least 50% of said plurality of particles comprise immobilized thereto a nucleic acid molecule of said plurality of nucleic acid molecules that is coupled to a single-stranded binding moiety of said plurality of single-stranded binding moieties.

124. The composition of embodiment 123, wherein at least 70% of said plurality of particles comprise immobilized thereto a nucleic acid molecule of said plurality of nucleic acid molecules that is coupled to a single-stranded binding moiety of said plurality of single-stranded binding moieties.

125. The composition of embodiment 124, wherein at least 90% of said plurality of particles comprise immobilized thereto a nucleic acid molecule of said plurality of nucleic acid molecules that is coupled to a single-stranded binding moiety of said plurality of single-stranded binding moieties.

126. The composition of any one of embodiments 120-125, wherein no more than 1% of particles of said plurality of particles are included in a particle aggregate comprising two or more particles of said plurality of particles.

127. The composition of embodiment 126, wherein no more than 0.1% of particles of said plurality of particles are included in a particle aggregate comprising two or more particles of said plurality of particles.

128. The composition of embodiment 127, wherein no more than 0.01% of particles of said plurality of particles are included in a particle aggregate comprising two or more particles of said plurality of particles.

129. The composition of any one of embodiments 120-128, wherein particle aggregates, comprising two or more particles of said plurality of particles, having a diameter of at least 5 micrometers (µm) are absent from said composition.

130. The composition of embodiment 129, wherein particle aggregates, comprising two or more particles of said plurality of particles, having a diameter of at least 1 micrometers (µm) are absent from said composition.

131. The composition of any one of embodiments 120-130, wherein particle aggregates, comprising two or more particles of said plurality of particles, having an area of at least 8 square micrometers (µm²) are absent from said composition.

132. The composition of any one of embodiments 97-131, wherein said plurality of particles comprises a particle that does not comprise a nucleic acid molecule coupled to a single-stranded binding moiety.

133. The composition of any one of embodiments 97-132, wherein said first particle comprises a first subset of said plurality of nucleic acid molecules immobilized thereto, wherein said first subset comprises said first nucleic acid molecule and has at least partial sequence identity to a first nucleic acid sequence.

134. The composition of embodiment 133, wherein said second particle comprises a second subset of said plurality of nucleic acid molecules immobilized thereto, wherein said second subset is different from said first subset, wherein said second subset has at least partial sequence identity to a second nucleic acid sequence.

135. The composition of embodiment 134, wherein said second nucleic acid sequence is different than said first nucleic acid sequence.

136. The composition of embodiment 134, wherein said second nucleic acid sequence and said first nucleic acid sequence are identical.

137. The composition of any one of embodiments 133-136, wherein said first subset of said plurality of nucleic acid molecules comprises at least 1,000 nucleic acid molecules.

138. The composition of embodiment 137, wherein said first subset of said plurality of nucleic acid molecules comprises at least 100,000 nucleic acid molecules.

139. The composition of any one of embodiments 97-108, wherein said plurality of particles comprises at least 10,000,000 particles.

140. The composition of embodiment 139, wherein said plurality of particles comprises at least 1,000,000,000 particles.

141. A system, comprising:
(i) a first solution comprising a suspension comprising a plurality of particles comprising a first particle and a second particle, wherein said plurality of particles comprises a plurality of nucleic acid molecules immobilized thereto, wherein said first particle comprises a first subset of said plurality of nucleic acid molecules immobilized thereto, wherein said first subset comprises a first nucleic acid molecule comprising a single-stranded portion, wherein said first subset of said plurality of nucleic acid molecules has at least partial sequence identity to a first nucleic acid sequence, wherein said second particle comprises a second subset of said plurality of nucleic acid molecules immobilized thereto, wherein said second subset is different from said first subset, wherein said second subset of said plurality of nucleic acid molecules has at least partial sequence identity to a second nucleic acid sequence different from said first nucleic acid sequence; and
(ii) a second solution comprising a single-stranded binding moiety configured to couple to said single-stranded portion of said first nucleic acid molecule.

142. The system of embodiment 141, wherein said plurality of particles is a plurality of beads.

143. The system of embodiment 141 or 142, wherein said plurality of nucleic acid molecules comprises a plurality of deoxyribonucleic acid (DNA) molecules, ribonucleic acid (RNA) molecules, or a combination thereof.

144. The system of any one of embodiments 141-143, wherein said single-stranded portion comprises single-stranded deoxyribonucleic acid (ssDNA), ribonucleic acid (RNA), or a combination thereof.

145. The system of any one of embodiments 141-144, wherein said first nucleic acid molecule comprises a sequence of a sample nucleic acid molecule, or a complement thereof.

146. The system of any one of embodiments 141-145, wherein said first nucleic acid molecule comprises a priming sequence, or a complement thereof.

147. The system of embodiment 146, wherein said priming sequence is a targeted priming sequence.

148. The system of embodiment 146, wherein said priming sequence comprises a random N-mer sequence.

149. The system of any one of embodiments 141-148, wherein said first nucleic acid molecule comprises a barcode sequence or a unique molecular identifier sequence.

150. The system of any one of embodiments 141-149, wherein said second solution comprises one or more reagents configured to facilitate coupling of said single-stranded binding moiety and said single-stranded portion.

151. The system of embodiment 150, wherein said one or more reagents comprises a salt.

152. The system of embodiment 149 or 150, wherein said one or more reagents comprises spermine.

153. The system of any one of embodiments 150-152, wherein said one or more reagents comprises cobalt hexammine.

154. The system of any one of embodiments 141-153, wherein said single-stranded binding moiety comprises a single-stranded binding (SSB) protein.

155. The system of embodiment 154, wherein said SSB protein is a T4 phage-derived SSB protein, an Escherichia coli-derived SSB protein, or an Extreme Thermostable SSB protein.

156. The system of any one of embodiments 141-155, wherein said single-stranded binding moiety comprises a second nucleic acid molecule.

157. The system of embodiment 156, wherein said second nucleic acid molecule comprises a random N-mer.

158. The system of embodiment 157, wherein N is between 6 and 12.

159. The system of embodiment 158, wherein said second nucleic acid molecule comprises 6 bases.

160. The system of embodiment 156, wherein said second nucleic acid molecule has sequence complementarity to a sequence of said single-stranded portion of said first nucleic acid molecule.

161. The system of any one of embodiments 156-160, wherein said single-stranded binding moiety comprises a loop structure.

162. The system of embodiment 161, wherein said loop structure is included in a hairpin moiety.

163. The system of embodiment 161, wherein said single-stranded binding moiety comprises a single-stranded nucleic acid molecule coupled to a moiety comprising said loop structure.

164. The system of any one of embodiments 141-163, wherein said second solution comprises a plurality of single-stranded binding moieties comprising said single-stranded binding moiety.

165. The system of embodiment 164, wherein said plurality of single-stranded binding moieties are of a same type.

166. The system of embodiment 164, wherein said plurality of single-stranded binding moieties comprises multiple types of single-stranded binding moieties.

167. The system of any one of embodiments 141-167, wherein said first subset of said plurality of nucleic acid molecules comprises at least 1,000 nucleic acid molecules.

168. The system of embodiment 167, wherein said first subset of said plurality of nucleic acid molecules comprises at least 100,000 nucleic acid molecules.

169. The system of any one of embodiments 141-168, wherein said plurality of particles comprises at least 10,000,000 particles.

170. The system of embodiment 169, wherein said plurality of particles comprises at least 1,000,000,000 particles.

171. A method for storing a solution comprising a plurality of particles, comprising:
(a) providing said solution comprising said plurality of particles, wherein said plurality of particles comprises a first set of nucleic acid molecules immobilized thereto, wherein said first set of nucleic acid molecules are configured to capture sample nucleic acid molecules derived from one or more nucleic acid samples;
(b) contacting said plurality of particles with a second set of nucleic acid molecules under conditions sufficient for at least 90% of first nucleic acid molecules of said first set of nucleic acid molecules to couple to second nucleic acid molecules of said second set of nucleic acid molecules, wherein said second set of nucleic acid molecules are not said sample nucleic acid molecules; and
(c) subsequent to (b), storing said solution for a time period of at least 1 hour.

172. The method of embodiment 171, wherein a first particle of said plurality of particles comprises a first subset of said first set of nucleic acid molecules immobilized thereto and a second particle of said plurality of particles comprises a second subset of said first set of nucleic acid molecules immobilized thereto, wherein said second subset is different from said first subset, wherein said first subset of said first set of nucleic acid molecules have at least partial sequence identity to a first nucleic acid sequence and said second subset of said first set of nucleic acid molecules have at least partial sequence identity to a second nucleic acid sequence.

173. The method of embodiment 172, wherein said first nucleic acid sequence is different from said second nucleic acid sequence.

174. The method of embodiment 172, wherein said first nucleic acid sequence and said second nucleic acid sequence are identical.

175. The method of any one of embodiments 171-174, wherein, during storage of said solution in (c), each first nucleic acid molecule of said first set of nucleic acid molecules that is hybridized to a second nucleic acid molecule of said second set of nucleic acid molecules does not hybridize to another nucleic acid molecule of said first set of nucleic acid molecules.

176. The method of any one of embodiments 171-175, wherein (b) comprises contacting said plurality of particles with said second set of nucleic acid molecules under conditions sufficient for at least 95% of first nucleic acid molecules of said first set of nucleic acid molecules to couple to said second nucleic acid molecules of said second set of nucleic acid molecules.

177. The method of any one of embodiments 171-176, wherein (c) comprises storing said solution at temperatures between about 18° C. to about 30° C.

178. The method of any one of embodiments 171-177, wherein (c) comprises storing said solution for at least 6 hours.

179. The method of embodiment 178, wherein (c) comprises storing said solution for at least 24 hours.

180. The method of embodiment 179, wherein (c) comprises storing said solution for at least 2 days.

181. The method of any one of embodiments 171-180, wherein a second nucleic acid molecule of said second set of nucleic acid molecules comprises a sequence that is substantially complementary to a sequence of said first set of nucleic acid molecules.

182. The method of embodiment 181, wherein said sequence of said first set of nucleic acid molecules comprises at least 6 bases.

183. The method of any one of embodiments 171-182, wherein each first nucleic acid molecule of said first set of nucleic acid molecules comprises a common nucleic acid sequence.

184. The method of any one of embodiments 171-183, wherein said first set of nucleic acid molecules comprises one or more different nucleic acid sequences.

185. The method of any one of embodiments 171-184, wherein said first set of nucleic acid molecules comprise a plurality of priming sequences.

186. The method of embodiment 185, wherein said plurality of priming sequences comprises a plurality of poly(T) sequences.

187. The method of embodiment 185, wherein said plurality of priming sequences comprises a plurality of random N-mer sequences.

188. The method of any one of embodiments 171-185, wherein said first set of nucleic acid molecules comprises a plurality of deoxyribonucleic acid (DNA) molecules, a plurality of ribonucleic acid (RNA) molecules, or a combination thereof.

189. The method of any one of embodiments 171-188, wherein said second set of nucleic acid molecules comprises deoxyribonucleic acid (DNA) nucleotides, ribonucleic acid (RNA) nucleotides, or a combination thereof.

190. The method of any one of embodiments 171-189, wherein each nucleic acid molecule of said second set of nucleic acid molecules comprises at least 6 bases.

191. The method of any one of embodiments 171-190, further comprising: (d) subsequent to (c), subjecting said plurality of particles to conditions sufficient to decouple said second nucleic acid molecules of said second set of nucleic acid molecules from said first nucleic acid molecules of said first set of nucleic acid molecules.

192. The method of embodiments 171-191, wherein (d) comprises denaturing said second nucleic acid molecules of said second set of nucleic acid molecules from said first nucleic acid molecules of said first set of nucleic acid molecules via application of a chemical or thermal stimulus.

193. The method of embodiment 192, wherein said chemical stimulus comprises sodium hydroxide.

194. The method of embodiment 192, wherein (d) comprises denaturing said second nucleic acid molecules of said second set of nucleic acid molecules from said first nucleic acid molecules of said first set of nucleic acid molecules via application of said thermal stimulus.

195. The method of embodiment 194, wherein a first nucleic acid molecule of said first set of nucleic acid molecules hybridized to a second nucleic acid molecule of said second nucleic acid molecules has a melting point between about 35° C. and 55° C.

196. The method of embodiment 191, wherein (d) comprises enzymatic degradation of said second nucleic acid molecules of said second set of nucleic acid molecules.

197. The method of any one of embodiments 191-196, further comprising, subsequent to (d), using first nucleic acid molecules of said first set of nucleic acid molecules immobilized to said plurality of particles for one or more applications selected from the group consisting of: hybridization capture of said sample nucleic acid molecules or derivatives thereof, single nucleotide polymorphism (SNP) genotyping of said sample nucleic acid molecules or derivatives thereof, sequencing library capture, synthesis of nucleic acid molecules, on-surface amplification of said sample nucleic acid molecules or derivatives thereof, and downstream processing or analysis of said sample nucleic acid molecules or derivatives thereof.

198. The method of any one of embodiments 171-197, wherein said plurality of particles are immobilized to a substrate.

199. The method of embodiment 198, wherein said plurality of particles is immobilized to a substantially planar array of said substrate.

200. The method of embodiment 198 or 196, wherein said plurality of particles is immobilized to said substrate at independently addressable locations.

201. The method of embodiment 200, wherein said independently addressable locations are substantially planar.

202. The method of embodiment 200, wherein said independently addressable locations comprise one or more wells.

203. The method of embodiment 200, wherein said independently addressable locations comprise one or more pillars.

204. The method of any one of embodiments 198-203, wherein said plurality of particles is immobilized to said substrate in a random pattern.

205. The method of any one of embodiments 198-204, wherein said plurality of particles is immobilized to said substrate in a predetermined pattern.

206. The method of any one of embodiments 198-205, wherein said plurality of particles is immobilized to said substrate with a density of at least 1,000 particles per mm².

207. A method for nucleic acid processing, comprising:
(a) providing a solution comprising:
   (i) a plurality of particles, wherein said plurality of particles comprises a first set of nucleic acid molecules immobilized thereto, wherein said first set of nucleic acid molecules are configured to capture sample nucleic acid molecules derived from one or more nucleic acid samples; and
   (ii) a second set of nucleic acid molecules, wherein said second set of nucleic acid molecules are not said sample nucleic acid molecules, and wherein said second set of nucleic acid molecules comprises sequences that are substantially complementary to sequences of said first set of nucleic acid molecules, wherein said solution has been stored for a time period of at least 1 hour under conditions sufficient for at least 90% of first nucleic acid molecules of said first set of nucleic acid molecules to couple to second nucleic acid molecules of said second set of nucleic acid molecules; and
(b) subjecting said plurality of particles to conditions sufficient to decouple said second nucleic acid molecules of said second set of nucleic acid molecules from said first nucleic acid molecules of said first set of nucleic acid molecules.

208. The method of embodiment 207, wherein a first particle of said plurality of particles comprises a first subset of said first set of nucleic acid molecules immobilized thereto and a second particle of said plurality of particles comprises a second subset of said first set of nucleic acid molecules immobilized thereto, wherein said second subset is different from said first subset, wherein said first subset of said first set of nucleic acid molecules have at least partial sequence identity to a first nucleic acid sequence and said second subset of said first set of nucleic acid molecules have at least partial sequence identity to a second nucleic acid sequence.

209. The method of embodiment 208, wherein said first nucleic acid sequence is different from said second nucleic acid sequence.

210. The method of embodiment 208, wherein said first nucleic acid sequence and said second nucleic acid sequence are identical.

211. The method of any one of embodiments 207-210, wherein, during said storage of said solution, each first nucleic acid molecule of said first set of nucleic acid molecules that is hybridized to a second nucleic acid molecule of said second set of nucleic acid molecules does not hybridize to another nucleic acid molecule of said first set of nucleic acid molecules.

212. The method of any one of embodiments 207-211, wherein said solution has been stored for a time period of at least 1 hour under conditions sufficient for at least 95% of first nucleic acid molecules of said first set of nucleic acid molecules to couple to second nucleic acid molecules of said second set of nucleic acid molecules.

213. The method of any one of embodiments 207-212, wherein, prior to (b), said solution has been stored at temperatures between about 18° C. to about 30° C.

214. The method of any one of embodiments 207-213, wherein, prior to (b), said solution has been stored for a time period of at least 6 hours.

215. The method of embodiment 214, wherein, prior to (b), said solution has been stored for a time period of at least 24 hours.

216. The method of embodiment 215, wherein, prior to (b), said solution has been stored for a time period of at least 2 days.

217. The method of any one of embodiments 207-216, wherein a second nucleic acid molecule of said second set of nucleic acid molecules comprises a sequence that is substantially complementary to a sequence of said first set of nucleic acid molecules.

218. The method of embodiment 217, wherein said sequence of said first set of nucleic acid molecules comprises at least 6 bases.

219. The method of any one of embodiments 207-218, wherein each first nucleic acid molecule of said first set of nucleic acid molecules comprises a common nucleic acid sequence.

220. The method of any one of embodiments 207-219, wherein said first set of nucleic acid molecules comprises one or more different nucleic acid sequences.

221. The method of any one of embodiments 207-220, wherein said first set of nucleic acid molecules comprises a plurality of priming sequences.

222. The method of embodiment 221, wherein said plurality of priming sequences comprises a plurality of poly(T) sequences.

223. The method of embodiment 222, wherein said plurality of priming sequences comprises a plurality of random N-mer sequences.

224. The method of any one of embodiments 207-223, wherein said first set of nucleic acid molecules comprises a plurality of deoxyribonucleic acid (DNA) molecules, a plurality of ribonucleic acid (RNA) molecules, or a combination thereof.

225. The method of any one of embodiments 207-224, wherein said second set of nucleic acid molecules comprises deoxyribonucleic acid (DNA) nucleotides, ribonucleic acid (RNA) nucleotides, or a combination thereof.

226. The method of any one of embodiments 207-225, wherein each nucleic acid molecule of said second set of nucleic acid molecules comprises at least 6 bases.

227. The method of any one of embodiments 207-226, wherein (b) comprises denaturing said second nucleic acid molecules of said second set of nucleic acid molecules from said first nucleic acid molecules of said first set of nucleic acid molecules via application of a chemical or thermal stimulus.

228. The method of embodiment 227, wherein said chemical stimulus comprises sodium hydroxide.

229. The method of embodiment 227, (b) comprises denaturing said second nucleic acid molecules of said second set of nucleic acid molecules from said first nucleic acid molecules of said first set of nucleic acid molecules via application of said thermal stimulus.

230. The method of embodiment 229, wherein a first nucleic acid molecule of said first set of nucleic acid molecules hybridized to a second nucleic acid molecule of said second nucleic acid molecules has a melting point between about 35° C. and 55° C.

231. The method of any one of embodiments 207-226, wherein (b) comprises enzymatic degradation of said second nucleic acid molecules of said second set of nucleic acid molecules.

232. The method of any one of embodiments 207-231, further comprising, subsequent to (b), using said first set of nucleic acid molecules immobilized to a surface for one or more applications selected from the group consisting of: hybridization capture of said sample nucleic acid molecules or derivatives thereof, single nucleotide polymorphism (SNP) genotyping of said sample nucleic acid molecules or derivatives thereof, sequencing library capture, synthesis of nucleic acid molecules, on-surface amplification of said sample nucleic acid molecules or derivatives thereof, and downstream processing or analysis of said sample nucleic acid molecules or derivatives thereof.

233. The method of any one of embodiments 207-232, wherein said plurality of particles are immobilized to a substrate.

234. The method of embodiment 233, wherein said plurality of particles is immobilized to a substantially planar array of said substrate.

235. The method of embodiment 233 or 234, wherein said plurality of particles is immobilized to said substrate at independently addressable locations.

236. The method of embodiment 235, wherein said independently addressable locations are substantially planar.

237. The method of embodiment 235, wherein said independently addressable locations comprise one or more wells.

238. The method of embodiment 235, wherein said independently addressable locations comprise one or more pillars.

239. The method of any one of embodiments 233-238, wherein said plurality of particles is immobilized to said substrate in a random pattern.

240. The method of any one of embodiments 233-238, wherein said plurality of particles is immobilized to said substrate in a predetermined pattern.

241. The method of any one of embodiments 233-240, wherein said plurality of particles is immobilized to said substrate with a density of at least 1,000 molecules per mm².

242. A kit, comprising:
(i) a first solution comprising a plurality of particles comprising a first set of nucleic acid molecules immobilized thereto, wherein said first set of nucleic acid molecules are configured to capture sample nucleic acid molecules derived from one or more nucleic acid samples; and
(ii) a second set of nucleic acid molecules, wherein said second set of nucleic acid molecules comprises one or more second nucleic acid molecules, which one or more second nucleic acid molecules are not said sample nucleic acid molecules, and wherein said second set of nucleic acid molecules comprises sequences that are substantially complementary to sequences of said first set of nucleic acid molecules such that, upon contacting said plurality of particles with said second set of nucleic acid molecules, at least 70% of first nucleic acid molecules of said first set of nucleic acid molecules couple to second nucleic acid molecules of said second set of nucleic acid molecules.

243. The kit of embodiment 242, wherein a first particle of said plurality of particles comprises a first subset of said first set of nucleic acid molecules immobilized thereto, and wherein a second particle of said plurality of particles comprises a second subset of said first set of nucleic acid molecules immobilized thereto, wherein said second subset is different from said first subset, wherein said first subset of said first set of nucleic acid molecules have at least partial identity to a first nucleic acid sequence and said second subset of said first set of nucleic acid molecules have at least partial sequence identity to a second nucleic acid sequence.

244. The kit of embodiment 243, wherein said first nucleic acid sequence is different from said second nucleic acid sequence.

245. The kit of embodiment 243, wherein said first nucleic acid sequence and said second nucleic acid sequence are identical.

246. The kit of any one of embodiments 242-245, wherein said second set of nucleic acid molecules are included in a second solution separate from said first solution.

247. The kit of any one of embodiments 242-245, wherein said second set of nucleic acid molecules are included in said solution.

248. The kit of any one of embodiments 242-247, wherein a second nucleic acid molecule of said second set of nucleic acid molecules comprises a sequence that is substantially complementary to a sequence of said first set of nucleic acid molecules.

249. The kit of embodiments 248, wherein said sequence of said first set of nucleic acid molecules comprises at least 6 bases.

250. kit of any one of embodiments 242-249, wherein each first nucleic acid molecule of said first set of nucleic acid molecules comprises a common nucleic acid sequence.

251. The kit of any one of embodiments 242-250, wherein said first set of nucleic acid molecules comprises one or more different nucleic acid sequences.

252. The kit of any one of embodiments 242-251, wherein said first set of nucleic acid molecules comprises a plurality of priming sequences.

253. The kit of embodiment 252, wherein said plurality of priming sequences comprises a plurality of poly(T) sequences.

254. The kit of embodiment 252, wherein said plurality of priming sequences comprises a plurality of random N-mer sequences.

255. The kit of any one of embodiments 242-254, wherein said first set of nucleic acid molecules comprises a plurality of deoxyribonucleic acid (DNA) molecules, a plurality of ribonucleic acid (RNA) molecules, or a combination thereof.

256. The kit of any one of embodiments 242-255, wherein said second set of nucleic acid molecules comprises deoxyribonucleic acid (DNA) nucleotides, ribonucleic acid (RNA) nucleotides, or a combination thereof.

257. The kit of any one of embodiments 242-256, wherein each nucleic acid molecule of said second set of nucleic acid molecules comprises at least 6 bases.

258. The kit of any one of embodiments 242-257, wherein said sequences of said first set of nucleic acid molecules comprise between about 6-20 bases.

259. The kit of any one of embodiments 242-258, wherein said sequences of said first set of nucleic acid molecules and said sequences of said second set of nucleic acid molecules have the same number of nucleotides.

260. The kit of any one of embodiments 242-259, wherein said sequences of said first set of nucleic acid molecules and said sequences of said second set of nucleic acid molecules have different numbers of nucleotides.

261. The kit of any one of embodiments 242-260, wherein said sequences of said first set of nucleic acid molecules comprises one or more different nucleic acid sequences.

262. The kit of any one of embodiments 242-260, wherein said sequences of said first set of nucleic acid molecules are identical.

263. The kit of any one of embodiments 242-260, further comprising a chemical stimulus configured to decouple said first nucleic acid molecules from said second nucleic acid molecules.

264. The kit of embodiment 263, wherein said chemical stimulus comprises sodium hydroxide.

265. The kit of any one of embodiments 242-264, wherein said plurality of particles is immobilized to a substrate.

266. The kit of embodiment 265, wherein said plurality of particles is immobilized to a substantially planar array of said substrate.

267. The kit of embodiment 265 or 266, wherein said plurality of particles is immobilized to said substrate at independently addressable locations.

268. The kit of embodiment 267, wherein said independently addressable locations are substantially planar.

269. The kit of embodiment 267, wherein said independently addressable locations comprise one or more wells.

270. The method of embodiment 267, wherein said independently addressable locations comprise one or more pillars.

271. The kit of any one of embodiments 265-270, wherein said plurality of particles is immobilized to said substrate in a random pattern.

272. The kit of any one of embodiments 265-270, wherein said plurality of particles is immobilized to said substrate in a predetermined pattern.

273. The kit of any one of embodiments 265-270, wherein said plurality of particles is immobilized to said substrate with a density of at least 1,000 particles per mm².

274. A method for dispensing a plurality of particles onto a substrate, comprising:
(a) incubating said plurality of particles with a first buffer solution, wherein said first buffer solution is substantially depleted of a cation, wherein each of at least a subset of said plurality of particles comprises a nucleic acid molecule immobilized thereto;
(b) loading said substrate with a second buffer solution, wherein said second buffer solution comprises said cation; and
(c) dispensing said plurality of particles onto said substrate to immobilize at least said subset of said plurality of particles onto a plurality of individually addressable locations on said substrate

275. The method of embodiment 274, wherein said cation comprises a divalent cation.

276. The method of embodiment 275, wherein said divalent cation comprises a magnesium ion or a calcium ion.

277. The method of embodiment 276, wherein said divalent cation comprises said magnesium ion.

278. The method of embodiment 277, wherein said magnesium ion comprises Mg²⁺.

279. The method of embodiment 276, wherein said divalent cation comprises said calcium ion.

280. The method of embodiment 277, wherein said calcium ion comprises Ca²⁺.

281. The method of any one of embodiments 274-280, wherein said first buffer solution is free of said cation.

282. The method of any one of embodiments 274-281, wherein said second buffer solution comprises at least about 5 millimolar (mM) of said cation.

283. The method of any one of embodiments 274-282, wherein said second buffer solution comprises at least about 10 millimolar (mM) of said cation.

284. The method of any one of embodiments 274-283, wherein said second buffer solution comprises at least about 25 millimolar (mM) of said cation.

285. The method of any one of embodiments 274-284, wherein said second buffer solution comprises at least about 50 millimolar (mM) of said cation.

286. The method of any one of embodiments 274-285, wherein said first buffer solution comprises a Tris buffer solution.

287. The method of embodiment 286, wherein said Tris buffer solution comprises about 10 (millimolar) mM of Tris.

288. The method of embodiment 286 or 287, wherein said Tris buffer solution has a pH of about 7.0.

289. The method of any one of embodiments 274-288, wherein said second buffer solution comprises a Tris buffer solution.

290. The method of embodiment 289, wherein said Tris buffer solution comprises about 10 (millimolar) mM of Tris.

291. The method of embodiment 289 or 290, wherein said Tris buffer solution has a pH of about 7.0.

292. The method of any one of embodiments 274-291, wherein said Tris buffer solution comprises about 0.055% Tergitol by volume.

293. The method of any one of embodiments 274-292, wherein said plurality of particles comprises at least about 100,000 particles.

294. The method of any one of embodiments 274-293, wherein said plurality of particles comprises at least about 10,000,000 particles.

295. The method of any one of embodiments 274-294, wherein said plurality of particles comprises at least about 1,000,000,000 particles.

296. The method of any one of embodiments 274-295, wherein, subsequent to (c), at least 100,000 particles are immobilized on said substrate.

297. The method of any one of embodiments 274-296, wherein, subsequent to (c), at least 1,000,000,000 particles are immobilized on said substrate.

298. The method of any one of embodiments 274-297, wherein said plurality of particles, subsequent to contacting said first buffer solution, comprises a concentration of at least about 100,000 particles per microliter (µL) in said first buffer solution.

299. The method of any one of embodiments 274-298, wherein said plurality of particles, subsequent to contacting said first buffer solution, comprises a concentration of at least about 1,000,000 particles per microliter (µL) in said first buffer solution.

300. The method of any one of embodiments 274-299, wherein said plurality of particles, subsequent to contacting said first buffer solution, comprises a concentration of at least about 20,000,000 particles per microliter (µL) in said first buffer solution.

301. The method of any one of embodiments 274-300, wherein said first buffer solution has a volume of less than about 1 milliliter (mL).

302. The method of any one of embodiments 274-301, wherein said first buffer solution has a volume of less than about 100 microliter (µL).

303. The method of any one of embodiments 274-302, wherein said first buffer solution has a volume of less than about 20 microliter (µL).

304. The method of any one of embodiments 274-303, wherein (a) comprises a first incubation time of at least about 0.5 minutes.

305. The method of any one of embodiments 274-304, further comprising, subsequent to (b), incubating said substrate with said second buffer solution.

306. The method of embodiment 305, wherein said incubating said substrate with said second buffer solution comprises a second incubation time of at least about 60 minutes.

307. The method of any one of embodiments 274-306, further comprising, subsequent to (b), forming a layer of said cation on said substrate.

308. The method of embodiment 307, wherein said layer has a thickness of at least about 10-20 micrometers (µm).

309. The method of any one of embodiments 274-308, wherein said plurality of individually addressable locations comprises at least about 100,000 locations.

310. The method of any one of embodiments 274-309, wherein at least about 60% of said plurality of independently addressable locations has at least one of said plurality of particles immobilized thereto.

311. The method of embodiments 274-310, wherein at least about 90% of said plurality of independently addressable locations has at least one of said plurality of particles immobilized thereto.

312. The method of any one of embodiments 274-311 wherein the center of each independently addressable location of said plurality of individually addressable locations is separated by fewer than about 10 micrometers (µm).

313. The method of any one of embodiments 274-312, wherein the center of each independently addressable location of said plurality of individually addressable locations is separated by fewer than about 5 micrometers (µm).

314. The method of any one of embodiments 274-313, wherein the center of each independently addressable location of said plurality of individually addressable locations is separated by about 1.8 micrometers (µm).

315. The method of any one of embodiments 274-314, wherein the center of each independently addressable location of said plurality of individually addressable locations is separated by about 1.5 micrometers (µm).

316. The method of any one of embodiments 274-315, wherein the center of each independently addressable location of said plurality of individually addressable locations is separated by about 1 micrometers (µm).

317. The method of any one of embodiments 274-315, wherein said plurality of independently addressable locations are substantially planar.

318. The method of any one of embodiments 274-315, wherein said plurality of independently addressable locations comprises one or more wells.

319. The method of any one of embodiments 274-315, wherein said plurality of independently addressable locations comprises one or more pillars.

320. The method of any one of embodiments 274-319, wherein said plurality of particles is a plurality of beads.

321. The method of any one of embodiments 274-320, wherein an average maximum dimension of said plurality of particles shrinks upon contacting said second buffer solution.

322. The method of any one of embodiments 274-321, wherein said average maximum dimension of said plurality of particles shrinks by at least 5% upon contacting said second buffer solution.

323. The method of any one of embodiments 274-322, wherein said nucleic acid molecule comprises deoxyribonucleic acid (DNA) nucleotides, ribonucleic acid (RNA) nucleotides, or a combination thereof.

324. The method of any one of embodiments 274-323, wherein said nucleic acid molecule comprises deoxyribonucleic acid (DNA) nucleotides.

325. The method of any one of embodiments 274-324, wherein said nucleic acid molecule comprises ribonucleic acid (RNA) nucleotides.

What is claimed is:

1-98. (canceled)

99. A method for processing a plurality of particles, comprising:
(a) providing a first particle of said plurality of particles comprising a first nucleic acid molecule immobilized thereto, wherein said first nucleic acid molecule comprises a first single-stranded portion;
(b) contacting said first nucleic acid molecule with a first single-stranded binding moiety under conditions sufficient to couple said first single-stranded binding moiety to said first single-stranded portion of said first nucleic acid molecule to yield a first treated particle comprising a first blocked nucleic acid molecule immobilized thereto, wherein said first blocked nucleic acid molecule comprises said first nucleic acid molecule coupled to said first single-stranded binding moiety; and
(c) providing said first treated particle in a solution comprising a second treated particle, wherein said second treated particle comprises a second nucleic acid molecule comprising a second single-stranded portion coupled to a second single-stranded binding moiety.

100. The method of claim 99, wherein said first nucleic acid molecule comprises a sequence of a sample nucleic acid molecule or a complement thereof, a priming sequence or a complement thereof, or a combination thereof.

101. The method of claim 99, wherein said first nucleic acid molecule comprises a barcode sequence or a unique molecular identifier sequence.

102. The method of claim 99, wherein (b) comprises providing a reaction mixture comprising said first single-stranded binding moiety.

103. The method of claim 102, wherein said reaction mixture comprises spermine.

104. The method of claim 102, wherein said reaction mixture comprises cobalt hexammine.

105. The method of claim 99, wherein said first single-stranded binding moiety comprises a single-stranded binding (SSB) protein.

106. The method of claim 99, wherein said first single-stranded binding moiety comprises a third nucleic acid molecule.

107. The method of claim 106, wherein said first single-stranded binding moiety comprises a loop structure.

108. The method of claim 99, wherein said first particle comprises a first plurality of nucleic acid molecules immobilized thereto, wherein said first plurality of nucleic acid molecules comprises said first nucleic acid molecule, and wherein each of said first plurality of nucleic acid molecules has at least partial sequence identity to a first nucleic acid sequence.

109. The method of claim 99, further comprising immobilizing said first particle to a substrate.

110. The method of claim 109 wherein said substrate comprises said solution.

111. The method of claim 99, wherein (b) comprises contacting said plurality of particles with a plurality of single-stranded binding moieties under conditions sufficient to couple said single-stranded binding moieties of said plurality of single-stranded binding moieties to single-stranded portions of nucleic acid molecules immobilized to particles of said plurality of particles to yield a plurality of treated particles comprising said first treated particle and said second treated particle.

112. The method of claim 111, wherein, subsequent to (b), particle aggregates comprising two or more treated particles of said plurality of treated particles and having a dimension of at least about 1 micrometer (µm) are absent from said plurality of treated particles.

113. The method of claim 111, wherein, subsequent to (b), no more than 1% of treated particles of said plurality of treated particles are included in a particle aggregate comprising two or more treated particles of said plurality of treated particles.

114. The method of claim 111, further comprising immobilizing said plurality of treated particles to a substrate.

115. The method of claim 99, further comprising, prior to (a), denaturing a doublestranded portion of said first nucleic acid molecule to yield said first single-stranded portion.

116. The method of claim 99, further comprising sequencing said first single-stranded portion of said first nucleic acid molecule, or a portion thereof.

117. A method for processing a plurality of particles, comprising:
(a) providing said plurality of particles, wherein each particle of at least a subset of said plurality of particles comprises a nucleic acid molecule of a plurality of nucleic acid molecules immobilized thereto, wherein each nucleic acid molecule of at least said subset of said plurality of nucleic acid molecules comprises a single-stranded portion; and
(b) contacting said plurality of particles with a plurality of single-stranded binding moieties under conditions sufficient for single-stranded binding moieties of said plurality of single-stranded binding moieties to couple to single-stranded portions of nucleic acid molecules of said at least said subset of said plurality of nucleic acid molecules,
wherein, subsequent to (b), said plurality of particles are included in a solution, and
wherein no more than 1% of particles of said plurality of particles are included in a particle aggregate comprising two or more particles of said plurality of particles.

118. The method of claim 117, wherein said plurality of single-stranded binding moieties comprises a plurality of single-stranded binding (SSB) proteins.
